(19) United States
(12) Patent Application Publication
(10) Pub. No.: US 2004/0259816 A1
(43) Pub. Date: Dec. 23, 2004

(54) Compositions comprising plant-derived polyphenolic compounds and inhibitors of reactive oxygen species and methods of using thereof

(76) Inventors: Stephen J. Pandol, Los Angeles, CA (US); Anna Gukovskaya, Agoura Hills, CA (US); Moussa Yazbeck, Fort Smith, AR (US); Guido Eibl, Los Angeles, CA (US); Laszlo G. Boros, Los Angeles, CA (US)

Correspondence Address: Suzannah K. Sundby, Esq., Smith, Gambrell & Russell, LLP, 1850 M Street, NW #800, Washington, DC, 20036, US

(21) Appl. No.: 10/824,597

(22) Filed: Apr. 15, 2004

Related U.S. Application Data
(63) Continuation-in-part of application No. 10/260,609, filed on Oct. 1, 2002.

(51) Int. Cl. A61K 31/7048; A61K 31/353; A61K 31/198

(52) U.S. Cl. 514/27; 514/456; 514/561; 514/733; 514/743

(57) ABSTRACT

Disclosed are methods for treating, preventing, or inhibiting diseases and disorders associated with NF-κB activation including proliferative diseases such as cancer and inflammatory diseases such as pancreatitis in a subject which comprises administering at least one polyphenolic compound and/or at least one inhibitor of reactive oxygen species to the subject. Also disclosed are pharmaceutical compositions comprising at least one polyphenolic compound and/or at least one inhibitor of reactive oxygen species. The polyphenolic compound may be derived or isolated from plants. In some embodiments, the polyphenolic compound is a flavonoid. In other embodiments, the polyphenolic compound is a non-flavonoid. Other methods and kits are also disclosed as well as pharmaceutical compositions.

### CROSS-REFERENCE TO RELATED APPLICATIONS

This application is a continuation-in-part of U.S. Serial application Ser. No. 10/260,609 filed 1 Oct. 2002, naming Stephen J. Pandol and Anna Gukovskaya as inventors, which is herein incorporated by reference in its entirety.

### STATEMENT REGARDING GOVERNMENT SPONSORED RESEARCH OR DEVELOPMENT

This invention was made with Government support of Grant No. 00-00776V-20071, awarded by the State of California. The Government has certain rights in this invention.

### BACKGROUND OF THE INVENTION

1. Field of the Invention

The present invention generally relates to plant-derived polyphenolic compounds, inhibitors of reactive oxygen species (ROS) and compositions thereof and methods for treating, preventing, or inhibiting diseases and disorders associated with NF-κB activation, such as pancreatic cancer and pancreatitis.

2. Description of the Related Art

Pancreatic cancer is the fifth leading cause of cancer death in the United States Cures for this type of cancer are unusual with the cancer recurring as metastatic disease in most cases after the removal of the primary tumor at surgery. See DiMagno et al. (1999) Gastroenterology 117:1464-1484; and Todd et al. (1999) Pancreatic Adenocarcinoma. TEXTBOOK OF GASTROENTEROLOGY. Philadelphia: Lippincott Williams & Wilkins, p. 2178-2192.

The development of tumors results from an imbalance between cell proliferation and cell death, apoptosis, and necrosis. See Thompson (1995) Science 267:1456-1462. Apoptosis is an active form of cell suicide characterized by a set of events including chromatin condensation, plasma membrane blebbing, cell shrinkage, DNA cleavage by specific endonucleases, and translocation of phosphatidylserine from the inner leaflet of the plasma membrane to the outer leaflet. See Thompson (1995); and Cohen (1993) Immunol. Today 14:126-130. Phosphatidylserine serves as a marker for macrophages to recognize apoptotic cells and phagocytize them.

There is increasing evidence that one of the major underlying defects in most cancers is an inhibition of normal apoptosis. See Thompson (1995) and Cohen (1993). Furthermore, treatments such as radiation and chemotherapy act to kill tumor cells and induce tumor shrinkage by causing apoptosis of cancer cells. Apoptosis can additionally be caused by removal of growth factors, the action of specific cytokines, i.e. TNFα, IL-1β, and Fas ligand, and detachment of cells from their extracellular matrix. Recent reports including our own indicate that some polyphenolic phytochemicals are capable of causing apoptosis in cancer cells. See Mouria et al. (2002) Int. J. Cancer 98(5):761-769; Hsieh & Wu (1999) Exp. Cell. Res. 249:109-115; Huang et al. (1999) Carcinogenesis 20:237-242; Islam et al. (2000) Biochem. Biophys. Res. Commun. 270:793-797; Sakagami et al. (2000) Anticancer Res. 20:271-277; Gupta et al. (2000) Toxicol. Appl. Pharmacol. 164(1):82-90; Li et al. (2000) Jpn J. Cancer Res. 91(1):34-40; Paschka et al. (1998) Cancer Lett. 130:1-7; Wang et al. (1999) Eur. J. Cancer 35:1517-1525; and Surh (1999) Cancer Lett. 140: 1-10.

Although induction of apoptosis appears to be a promising therapeutic approach to the treatment of cancer, the intracellular mechanisms of apoptosis are incompletely understood. Thus, a need still exists for compositions and methods for inducing apoptosis and treating cancer.

### SUMMARY OF THE INVENTION

In some embodiments, the present invention relates to a method of treating, preventing, or inhibiting cancer in a subject comprising administering at least one polyphenolic compound and at least one inhibitor of reactive oxygen species to the subject. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the inhibitor is diphenylene iodonium, N-acetylcysteine, or Tiron. In some embodiments, the method further comprises administering at least one antioxidant to the subject. Preferably, the subject is mammalian, more preferably, the subject is human.

In some embodiments, the present invention provides a method of inducing apoptosis in a tumor comprising contacting the tumor with at least one polyphenolic compound. In some embodiments, the method further includes contacting the tumor with at least one inhibitor of reactive oxygen species. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the inhibitor is diphenylene iodonium, N-acetylcysteine, or Tiron. In some embodiments, the method further comprises contacting the tumor with at least one antioxidant. In some embodiments, the tumor is a primary tumor. In other embodiments, the tumor is metastatic.

In some embodiments, the present invention provides a method of activating caspase-3 with at least one polyphenolic compound. In some embodiments, the method further includes contacting the protein target with at least one inhibitor of reactive oxygen species. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the inhibitor is diphenylene iodonium, N-acetylcysteine, or Tiron. In some embodiments, the method further comprises contacting the protein target with at least one antioxidant.

In some embodiments, the present invention provides a method of activating caspase-3 with at least one polyphenolic compound and at least one inhibitor of reactive oxygen species. In some embodiments, the method further includes contacting caspase-3 with at least one inhibitor of reactive oxygen species. The polyphenolic compound may be derived or isolated from food. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the inhibitor is diphenylene iodonium, N-acetylcysteine, or Tiron. In some embodiments, the method further comprises contacting caspase-3 with at least one antioxidant.

In some embodiments, the present invention provides a method of preventing, inhibiting, or modulating NF-κB activation in a cell comprising administering to the cell at least one polyphenolic compound and MG-132, diphenylene iodonium, or both. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the method further comprises administering to the cell at least one antioxidant, at least one proteosomal inhibitor, or both.

In some embodiments, the present invention provides a method of making a cancer cell susceptible to apoptosis induced by a polyphenolic compound comprising inhibiting NF-κB activity in the cell. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol.

In some embodiments, the present invention provides a method of preventing, inhibiting, or attenuating the activation of Akt/PKB in a cell comprising administering to the cell at least one polyphenolic compound, at least one inhibitor of reactive oxygen species and at least one PI 3-kinase inhibitor, or at least one polyphenolic compound and at least one inhibitor of NADPH oxidase or at least one inhibitor of reactive oxygen species. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the inhibitor is diphenylene iodonium, N-acetylcysteine, or Tiron. In some embodiments, the method further comprises contacting the cell with at least one antioxidant.

In some embodiments, the present invention provides a pharmaceutical composition comprising at least one polyphenolic compound, at least one inhibitor of reactive oxygen species, and a pharmaceutically acceptable carrier. In some embodiments, the pharmaceutical composition may further comprise at least one antioxidant. In some embodiments, the pharmaceutical composition may further comprise at least one anti-neoplastic agent. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the inhibitor is diphenylene iodonium, N-acetylcysteine, or Tiron.

In some embodiments, the present invention provides a kit for treating, preventing, or inhibiting cancer which comprises at least one polyphenolic compound, at least one inhibitor of reactive oxygen species, and instructions for use. In some embodiments, the kit may further comprise at least one antioxidant. In some embodiments, the kit may further comprise at least one anti-neoplastic agent. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the inhibitor is diphenylene iodonium, N-acetylcysteine, or Tiron.

In some embodiments, the present invention relates to a method of depolarizing a mitochondrial membrane comprising contacting the mitochondrial membrane with at least one polyphenolic compound and at least one inhibitor of reactive oxygen species. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the inhibitor is diphenylene iodonium, N-acetylcysteine, or Tiron. In some embodiments, the method further comprises contacting the mitochondrial membrane with at least one antioxidant.

In some embodiments, the present invention relates to a method of activating mitochondrial permeability transition pore (PTP) comprising contacting the mitochondrial PTP with at least one polyphenolic compound and at least one inhibitor of reactive oxygen species. The polyphenolic compound may be derived or isolated from plants. In some embodiments, the polyphenolic compound is a flavonoid. In other embodiments, the polyphenolic compound is a non-flavonoid. In some preferred embodiments, the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid, rottlerin and traxol. Preferably, the polyphenolic compound is quercetin, rutin, genistein, curcumin, rottlerin or trans-resveratrol. In some embodiments, the inhibitor is diphenylene iodonium, N-acetylcysteine, or Tiron. In some embodiments, the method further comprises contacting the mitochondiral PTP with at least one antioxidant.

In some embodiments, the present invention provides a method of treating, preventing, inhibiting, or modulating NF-κB activation in a cell or in a subject which comprises administering at least one polyphenolic compound, an inhibitor of PKC 6 translocation, an inhibitor of PKC ε translocation, or a combination thereof to the cell or the subject. In some embodiments, the polyphenolic compound is rottlerin or a derivative thereof. In some embodiments, the method further comprises administering a second polyphenolic compound to the cell or the subject. In some embodiments, the second polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid and traxol. In some preferred embodiments, the second polyphenolic compound is quercetin, rutin, genistein, curcumin or trans-resveratrol. In some embodiments, the method further comprises administering at least one inhibitor of a reactive oxygen species to the cell or the subject. In some embodiments, the inhibitor is diphenylene iodonium, N-acetylcysteine, or Tiron. In some embodiments, the method further comprises administering at least one antioxidant to the cell or the subject. In some embodiments, the inhibitor of PKC δ translocation or the inhibitor of PKC ε translocation is a peptide. In some preferred embodiments, the peptide is δV1-1 or εV1-2.

The present invention also provides a method of treating, preventing, or inhibiting a disease or disorder associated with NF-κB activation in a subject which comprises treating, preventing, inhibiting, or modulating NF-κB activation in a cell or in a subject which comprises administering at least one polyphenolic compound, an inhibitor of PKC δ translocation, an inhibitor of PKC ε translocation, or a combination thereof to the cell or the subject. In some embodiments, the polyphenolic compound is rottlerin or a derivative thereof. In some embodiments, the method further comprises administering a second polyphenolic compound to the cell or the subject. In some embodiments, the second polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid and traxol. In some preferred embodiments, the second polyphenolic compound is quercetin, rutin, genistein, curcumin or trans-resveratrol. In some embodiments, the method further comprises administering at least one inhibitor of a reactive oxygen species to the cell or the subject. In some embodiments, the inhibitor is diphenylene iodonium, N-acetylcysteine, or Tiron. In some embodiments, the method further comprises administering at least one antioxidant to the cell or the subject. In some embodiments, the inhibitor of PKC δ translocation or the inhibitor of PKC ε translocation is a peptide. In some preferred embodiments, the peptide is δV1-1 or εV1-2. In some embodiments, the disease or disorder is a cancer, preferably pancreatic cancer, breast cancer, ovarian cancer, prostate cancer, kidney cancer, pancreatic cancer, colon cancer, thyroid cancer, melanoma, Hodgkin's lymphoma, acute lymphoblastic leukemia, acute myelogenous leukemia, diffuse large B-cell lymphoma, astrocytoma, glioblastoma, a head or neck cancer, or vulva cancer. In some embodiments, the cancer is related to in vitro transformation of BCR-ABL, DBL/DBS, RAF, RAS, TEL-JAK2, or TEL-PDGFR. In some embodiments, the cancer is related to viral oncogenesis caused by Epstein-Barr virus, hepatitis B virus, human herpesvirus-8, or human T-cell leukemia virus-1. In some embodiments, the disease or disorder is an inflammatory disease, preferably pancreatitis, inflammatory bowel disease, asthma, arthritis, rheumatoid arthritis, asthma, psoriasis, cystitis, or nephritis. In some embodiments, the disease or disorder is viral hepatitis, alcoholic liver disease, lung inflammation, Alzheimer's Disease, or atherosclerosis. In some embodiments, the method further comprises administering at least one antiproliferative agent, at least one anti-inflammatory agent, or both. In some embodiments, administration of the polyphenolic compound, the inhibitor of PKC δ translocation, the inhibitor of PKC ε translocation, or the combination thereof causes, induces, increases, or modulates cell cycle arrest, apoptosis, mitochondrial cytochrome c release, dissipation of mitochondrial polarity, caspase activation, mitochondrial permeability transition pore activation, or a combination thereof, in the cancer. In some embodiments, the method further comprises administering a second polyphenolic compound, at least one inhibitor of reactive oxygen species, at least one inhibitor of PI 3-kinase, at least one inhibitor of NADPH oxidase, or a combination thereof. In some embodiments, administration of the second polyphenolic compound, the inhibitor of reactive oxygen species, the inhibitor of PI 3-kinase, the inhibitor of NADPH oxidase, or the combination thereof causes, induces, increases, or modulates cell cycle arrest, apoptosis, mitochondrial cytochrome c release, dissipation of mitochondrial polarity, caspase activation, mitochondrial permeability transition pore activation, or a combination thereof, in the cancer. In some embodiments, rottlerin inhibits non-oxidative deoxyribose synthesis, inhibits nucleic acid synthesis, induces cell cycle arrest, inhibits cell proliferation, increases oxidative metabolism of glucose, inhibits de novo fatty acid synthesis, chain elongation and desaturation from glucose, or a combination thereof, in the cancer.

In some embodiments, the present invention provides a method of inducing apoptosis in a cell or making the cell susceptible to apoptosis which comprises treating, preventing, inhibiting, or modulating NF-κB activation in a cell or in a subject which comprises administering at least one polyphenolic compound, an inhibitor of PKC δ translocation, an inhibitor of PKC ε translocation, or a combination thereof to the cell or the subject. In some embodiments, the polyphenolic compound is rottlerin or a derivative thereof. In some embodiments, the method further comprises administering a second polyphenolic compound to the cell or the subject. In some embodiments, the second polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid and traxol. In some preferred embodiments, the second polyphenolic compound is quercetin, rutin, genistein, curcumin or trans-resveratrol. In some embodiments, the method further comprises administering at least one inhibitor of a reactive oxygen species to the cell or the subject. In some embodiments, the inhibitor is diphenylene iodonium, N-acetylcysteine, or Tiron. In some embodiments, the method further comprises administering at least one antioxidant to the cell or the subject. In some embodiments, the inhibitor of PKC δ translocation or the inhibitor of PKC ε translocation is a peptide. In some preferred embodiments, the peptide is δV1-1 or εV1-2. In some embodiments, the cell is a tumor cell or a cancer cell. In some embodiments, the tumor is a primary tumor. In some embodiments, the tumor is metastatic.

In some embodiments, the present invention provides a method of inhibiting non-oxidative deoxyribose synthesis, inhibiting nucleic acid synthesis, inducing cell cycle arrest, inhibiting cell proliferation, increasing oxidative metabolism of glucose, inhibiting de novo fatty acid synthesis, chain elongation and desaturation from glucose, or a combination thereof, in a cell which comprises contacting the cell with rottlerin.

In some embodiments, the present invention provides a purified polypeptide comprising SFNSYELGSLRQIKIWFQNRRMKWKK (SEQ ID NO:10), EAVSLKPTRQIKIWFQNRRMKWKK (SEQ ID NO:11), or LSETKPAVRQIKIWFQNRRMKWKK (SEQ ID NO:12).

In some embodiments, the present invention provides a pharmaceutical composition comprising rottlerin or a derivative thereof and a pharmaceutically acceptable carrier. In some embodiments, the pharmaceutical composition further comprises a second polyphenolic compound. In some embodiments, the second polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid and traxol. In some preferred embodiments, the second polyphenolic compound is quercetin, rutin, genistein, curcumin or trans-resveratrol. In some embodiments, the pharmaceutical composition further comprises an inhibitor of PKC 8 translocation, an inhibitor of PKC ε translocation, or both. In some embodiments the inhibitor of PKC 8 translocation or the inhibitor of PKC ε translocation is SFNSYELGSLRQIKIWFQNRRMKWKK (SEQ ID NO:10) or EAVSLKPTRQIKIWFQNRRMKWKK (SEQ ID NO:11). In some embodiments, the pharmaceutical composition further comprises an inhibitor of a reactive oxygen species. In some embodiments, the pharmaceutical composition further comprises an antioxidant.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention and together with the description serve to explain the principles of the invention.

### DESCRIPTION OF THE DRAWINGS

FIG. 1 illustrates that serum and insulin growth factor-1 (IGF-1) stimulate the production of ROS in Mia PACA-2 and PANC-1 pancreatic cancer cells. Mia PACA-2 cells (A); PANC-1 cells (B). Values are means±SE (n=3). *p<0.05 compared to cells cultured without serum or IGF-1.

FIG. 2A shows the results of intracellular H₂O₂ measured by flow cytometry of DCF-labeled cells. DPI=diphenylene iodonium; RS=trans-resveratrol; Gen=genistein.

FIG. 2B shows the percentage of cells with high DCF fluorescence. Values are means±SE (n=3). *p<0.05 compared to control cells. DPI=diphenylene iodonium; RS=trans-resveratrol; Gen=genistein.

FIG. 3A is a gel eletrophoresis that shows that quercetin and trans-resveratrol, but not rutin, caused an increase in oligonucleosomal DNA fragmentation. RN=rutin, Q=quercetin, RS=trans-resveratrol.

FIG. 3B is a graph showing that quercetin is more potent in causing apoptosis than trans-resveratrol. The values represent means±SE (n=3). *p<0.05 compared to control cells. RN=rutin, Q=quercetin, RS=trans-resveratrol.

FIG. 3C are Western blots that illustrate that poly(ADP-ribose) polymerase (PARP) was cleaved in cell lines treated with quercetin and trans-resveratrol, but not rutin. RN=rutin, Q=quercetin, RS=trans-resveratrol.

FIG. 4 shows that the combination of inhibitors of production of ROS and polyphenolic compounds cause oligonucleosomal DNA fragmentation in Mia PACA-2 pancreatic cancer cells. DPI=diphenylene iodonium; RS=trans-resveratrol; Gen=genistein. Values are means±SE (n=5). *p<0.05 compared to control cells. #p<0.05 compared to cells treated with polyphenols only, or antioxidants only.

FIG. 5 shows the effects of inhibition of ROS and trans-resveratrol on Annexin V staining in Mia PACA-2 pancreatic cancer cells. PI=propidium iodide; AnV=Annexin V; RS=trans-resveratrol; Gen=genistein. Values are means±SE (n=3) *p<0.05 compared to control cells. #p<0.05 compared to cells treated with resveratrol only, or tiron only.

FIG. 6A is a Western blot showing that trans-resveratrol converts caspase-3 into the active form in BSp73AS cells.

FIG. 6B shows the results of a fluorogenic assay that confirms that trans-resveratrol and quercetin activate caspase-3 in a time dependent manner in BSp73AS cells. The values represent means±SE (n=3). *p<0.05 compared to control cells. RN=rutin, Q=quercetin, RS=trans-resveratrol.

FIG. 6C shows the results of a fluorogenic assay that confirms that quercetin activates caspase-3 in a dose dependent manner in BSp73AS cells. The values represent means±SE (n=3). *p<0.05 compared to control cells. RN=rutin, Q=quercetin, RS=trans-resveratrol.

FIG. 7A is a Western blot showing that quercetin converts caspase-3 into the active form in Mia PACA-2 cells.

FIG. 7B shows the results of a fluorogenic assay that confirms that trans-resveratrol and quercetin activate caspase-3 in a time dependent manner in Mia PACA-2 cells. The values represent means±SE (n=3). *p<0.05 compared to control cells. RN=rutin, Q=quercetin, RS=trans-resveratrol.

FIG. 8A shows the results of a fluorimetric assay providing that Tiron, polyphenolic compounds and a caspase inhibitor have an effect on caspase-3 activity in Mia PACA-2 pancreatic cancer cells. Z-VAD=z-VAD.fmk; DPI=diphenylene iodonium; RS=trans-resveratrol; Gen=genistein. Values are means±SE (n=3). *p<0.05 compared to control cells or cells treated with DPI only or polyphenolic compounds only. Values are means±SE (n=5).

FIG. 8B shows that Tiron, polyphenolic compounds and a caspase inhibitor have an effect on oligonucleosomal DNA fragmentation in Mia PACA-2 pancreatic cancer cells. *p<0.05 compared to control cells. #p<0.05 compared to cells treated with polyphenols only, or antioxidants only. Ap<0.05 compared to the values obtained in the absence of Z-VAD.

FIG. 9A is a Western blot showing that polyphenolic compounds stimulate mitochondrial release of cytochrome c in BSp73AS cells. RN=rutin, Q=quercetin, RS=trans-resveratrol, GN=genistein.

FIG. 9B is a Western blot showing that polyphenolic compounds stimulate mitochondrial release of cytochrome c in Mia PACA-2 cells. RN=rutin, Q=quercetin, RS=trans-resveratrol, GN=genistein.

FIG. 10A shows that polyphenolic compounds induce depolarization of mitochondrial membrane potential in BSp73AS cells. The values represent means±SE (n=3). *p<0.05 compared to control cells. RN=rutin, Q=quercetin, RS=trans-resveratrol, GN=genistein.

FIG. 10B shows that polyphenolic compounds induce depolarization of mitochondrial membrane potential in Mia PACA-2 cells. The values represent means±SE (n=3). *p<0.05 compared to control cells. RN=rutin, Q=quercetin, RS=trans-resveratrol, GN=genistein.

FIG. 11A shows the effects of DPI, and polyphenolic compounds on mitochondrial membrane potential in Mia PACA-2 pancreatic cancer cells. Changes in Δψₘ as measured by FACS® in cells labeled with DiOC₆(3). Values are means±SE; (n=3).

FIG. 11B shows the percentage of cells with high Δψₘ. Values are means±SE; (n=3).

FIG. 12A shows that inhibition of PTP by cyclosporin A alone or in combination with aristolochic acid prevents release of mitochondrial cytochrome c release in MiaPACA-2 cancer cells treated with trans-resveratrol, quercetin and genistein. Z-VAD prevents mitochondrial cytochrome c release in untreated (control) cells. Q=quercetin, RS=trans-resveratrol, GN=genistein.

FIG. 12B shows that inhibition of PTP by cyclosporine A and aristolochic acid attenuates caspase-3 activity in Mia PACA-2 cells treated with polyphenolic compounds. The caspase inhibitor Z-VAD blocked caspase activity in the MiaPACA-2 cells. Q=quercetin, RS=trans-resveratrol, GN=genistein. The values represent means±SE (n=3). *p<0.05 compared to cells treated with the polyphenolic compound alone.

FIG. 12C shows that inhibition of PTP by cyclosporin A and aristolochic acid and inhibition of caspases by Z-VAD decreases apoptosis in MiaPACA-2 cells treated with polyphenolic compounds. Q=quercetin, RS=trans-resveratrol, GN=genistein. The values represent means±SE (n=3). *p<0.05 compared to untreated cells.

FIG. 13A is an immunoblot that shows the effect of polyphenolic compounds alone and in combination on cytochrome c release in Mia PACA-2 cells. Q=quercetin, RS=trans-resveratrol.

FIG. 13B shows the effect of polyphenolic compounds alone and in combination on caspase-3 activity in Mia PACA-2 cells. Q=quercetin, RS=trans-resveratrol. The values for trans-resveratrol, quercetin and the combination represent the means±SE (n=3) with the values for the controls subtracted. The dashed line over the bar for the values for trans-resveratrol plus quercetin represents the “predicted” additive values for the response to both agents. The recorded values were statistically significantly greater than the “predicted” additive values (p<0.05).

FIG. 14A shows that NF-κB is constitutively active in both BSp73As and Mia PACA-2 cells. Positions of specific NF-κB-DNA complexes and the free probe are indicated by single and double arrowheads, respectively. RN=rutin, Q=quercetin, RS=trans-resveratrol, GN=genistein, MG=MG-132.

FIG. 14B shows the relative NF-κB activities in cells treated with polyphenolic compounds or MG-132. The values represent densitometric intensities of NF-κB band quantified with Phosphorlmager, relative to cells not treated with polyphenolic compounds or NF-κB inhibitors. Values represent means±SE (n=3). *p<0.05 as compared to control cells. RN=rutin, Q=quercetin, RS=trans-resveratrol, GN=genistein, MG=MG-132.

FIG. 14C shows caspase-3 activity and annexin staining in BSp73AS and Mia PACA-2 cells. The values represent means±SE (n=3). The results for caspase-3 were normalized to the DEVDase activity in untreated cells. *p<0.05 as compared to control cells. RN=rutin, Q=quercetin, RS=trans-resveratrol, GN=genistein, MG=MG-132.

FIG. 15 shows the effects of combinations of DPI and polyphenolic compounds on NF-κB activation in the Mia PACA-2 pancreatic cancer cells. DPI=diphenylene iodonium; RS=trans-resveratrol; GN=genistein.

FIG. 16 shows the effects of MG-132, DPI and trans-resveratrol on oligonucleosomal DNA fragmentation in Mia PACA-2 pancreatic cancer cells. DPI=diphenylene iodonium; RS=trans-resveratrol; MG=MG-132. Values are means±SE (n=4). *p<0.05 compared to control cells. #p<0.05 compared to cells treated with MG-132 only. Ap<0.05 compared to cells treated with MG-132+DPI.

FIG. 17 shows the effects of serum, LY294002 and genistein on Akt/PKB phosphorylation in Mia PACA-2 pancreatic cancer cells. GN=genistein. The upper panels show representative Western blots performed on whole cell lysates using an antibody against phosphorylated Akt/PKB. The membranes were then stripped and re-probed with an antibody against total Akt (lower panel).

FIG. 18 shows the effects of LY294002 and DPI on NF-κB activation in Mia PACA-2 pancreatic cancer cells. DPI=diphenylene iodonium.

FIG. 19A shows subcellular distribution of PKC isoforms in response to CCK-8 in rat pancreatic acini. Shown are representative Western blots from 3 independent experiments.

FIG. 19B shows changes in PKC kinase activities stimulated by CCK-8 in rat pancreatic acini. For each PKC isoform, activity values were normalized on its basal activity in unstimulated control acini. Values are means±SE (n=5-10). *p<0.05 compared to each isoform basal activity.

FIG. 20A shows NF-κB binding activity by electromobility shift assay (EMSA) in rat pancreatic acini treated with or without CCK-8 in the presence or absence of inhibitors of specific isoforms of PKC.

FIG. 20B shows NF-κB band intensities rat pancreatic acini treated with or without CCK-8 in the presence or absence of inhibitors of isoforms of PKC in unstimulated control acini. Values are means±SE (n=5). *p<0.05 compared to unstimulated control. #p<0.05 compared to CCK-8 alone.

FIG. 20C shows IκBα degradation in cytosolic extracts of rat pancreatic acini treated with or without CCK-8 in the presence of absence of inhibitors of specific isoforms of PKC. Representative of 5 independent experiments.

FIG. 21A shows cytosolic and membrane fractions of rat pancreatic acini treated with or without CCK-8 in the presence of absence of a specific translocation inhibitor of PKC δ (δV1-1) or a specific translocation inhibitor for PKCε (εV1-2) and subjected to SDS-PAGE and blotted using antibodies specific for PKC δ or E. Shown are representative blots from 3 independent experiments.

FIG. 21B1-2 show the effects of PKC translocation inhibitors on kinase activity in rat pancreatic acini. For each PKC isoform, activity values were normalized on its basal activity in unstimulated control acini. Values are means±SE (n=5-10). *p<0.05 compared to unstimulated control. #p<0.05 compared to CCK-8 alone.

FIG. 21C1-2 show pancreatic acini preincubated with the indicated concentration of δV1-1 (21 C₁) or εV1-2 (21C2) for 3 hours, and then stimulated with 100 nM CCK-8 for 30 minutes. The kinase activity responded to CCK-8 in the absence of the inhibitors was considered as 100%. Values are means±SE (n=3-10). *p<0.05 compared to CCK-8 alone.

FIG. 22A shows subcellular distribution of PKC isoforms in response to TNF-α in rat pancreatic acini. Shown are representative blots from 3 independent experiments.

FIG. 22B shows changes in PKC kinase activities stimulated by TNF-α in rat pancreatic acini. For each PKC isoform, activity values were normalized on its basal activity in unstimulated control acini. Values are means±SE (n=3-5). *p<0.05 compared to each isoform basal activity.

FIG. 23A shows NF-κB binding activity in nuclear extracts from rat pancreatic acini treated with or without TNF-α in the presence or absence of inhibitors of specific isoforms of PKC.

FIG. 23B shows NF-κB band intensities in rat pancreatic acini treated with or without TNF-α in the presence or absence of inhibitors of isoforms of PKC. Values are means±SE (n=4). *p<0.05 compared to unstimulated control. #p<0.05 compared to TNF-α alone.

FIG. 23C shows IκBα degradation in cytosolic extracts by Western blot analysis of rat pancreatic acini treated with or without TNF-α in the presence of absence of inhibitors of specific isoforms of PKC. Representative of 4 independent experiments.

FIG. 24A shows NF-κB binding activity in nuclear extracts from rat pancreatic acini treated with or without CCK-8 or TNF-α in the presence or absence of a Src kinase inhibitor, PP2.

FIG. 24B shows IκBα degradation in cytosolic extracts by Western blot analysis of rat pancreatic acini treated with or without CCK-8 or TNF-α in the presence or absence of a Src kinase inhibitor, PP2. Representative of 4 independent experiments.

FIG. 25 shows the effects of Src kinase inhibitor on tyrosine phosphorylation of PKC δ in rat pancreatic acini treated with or without CCK-8 or TNF-α. Representative of 4 independent experiments.

FIG. 26A shows NF-κB binding activity in nuclear extracts from rat pancreatic acini treated with or without CCK-8 or TNF-α in the presence or absence of a phosphatidylinositol (PI)-specific phospholipase C (PLC) inhibitor, U-73122, or a phosphatidylcholine (PC)-specific PLC inhibitor, D-609.

FIG. 26B shows IκBα degradation in cytosolic extracts by Western blot analysis of rat pancreatic acini treated with or without CCK-8 or TNF-α in the presence or absence of U-73122 or D-609. Representative of 3 independent experiments.

FIG. 27A1-2 shows effects of PLC inhibitors on PKC subcellular distribution induced by CCK-8 in rat pancreatic acini. Shown are representative blots from 3 independent experiments.

FIG. 27B1-2 shows effects of PLC inhibitors on PKC subcellular distribution induced by TNF-α in rat pancreatic acini. Shown are representative blots from 3 independent experiments.

FIG. 28A is a schematic of the signaling pathways involved in NF-κB activation induced by CCK-8 and TNF-α in pancreatic acinar cells.

FIG. 28B shows NF-κB binding activity in nuclear extracts from rat pancreatic acini treated with or without ethanol and CCK-8 in the presence or absence of rottlerin. Shown are representative blots from 3 independent experiments.

FIG. 29 shows rottlerin but not protein kinase C inhibitors cause apoptosis as measured by oligonucleosomal DNA fragmentation in MIA PaCa-2 pancreatic cancer cells.

FIG. 30 shows rottlerin but not protein kinase C inhibitors cause apoptosis as measured by oligonucleosomal DNA fragmentation in PANC-1 pancreatic cancer cells.

FIG. 31 shows the effects of rottlerin on apoptosis and necrosis as measured by Annexin V (AnV) and propidium iodide (PI) staining in MIA PaCa-2 pancreatic cancer cells.

FIG. 32 shows the effects of rottlerin and a caspase inhibitor (ZVAD) on caspase-3 activity (DEVDase activity) and oligonucleosomal DNA fragmentation in MIA PaCa-2 pancreatic cancer cells.

FIG. 33A1-4 are histograms that show changes in Δψm induced by rottlerin as measured by flow cytometry using a mitochondrial potential sensitive probe in MIA PaCa-2 pancreatic cancer cells.

FIG. 33B shows the effect of rottlerin on the percentage of MIA PaCa-2 pancreatic cancer cells with high Δψm.

FIG. 34 shows the effects of rottlerin on mitochondrial cytochrome c release in MIA PaCa-2 pancreatic cancer cells.

FIG. 35 shows the effects of rottlerin and GF109203X on NF-κB activation in MIA PaCa-2 pancreatic cancer cells.

FIG. 36A1-4 are histograms of intracellular H₂O₂ as measured by flow cytometry using an H₂O₂-sensitive intracellular probe (DCF) showing the effects of rottlerin (Rt) and GF109203X (GF) on production of ROS in MIA PaCa-2 pancreatic cancer cells.

FIG. 36B shows the percentage of cells with high DCF fluorescence.

FIG. 37 shows the effect of rottlerin on the growth of MIA PaCa-2 tumors in nude mice.

FIG. 38 shows the effect of rottlerin on deoxyribose (1) and ribose (2) 1³C tracer accumulation from glucose in MIA PaCa-2 cells.

FIG. 39 shows the effect of rottlerin on oxidative deoxyribose (1) and non-oxidative deoxyribose (2) synthesis, as well as oxidative ribose (3) and non-oxidative ribose (4) synthesis based on positional 1³C tracer accumulation from glucose into nucleic acid of MIA PaCa-2 cells.

FIG. 40 shows the effect of rottlerin on direct glucose oxidation and recycling in the pentose cycle in MIA PaCa-2 cells.

FIG. 41 shows the effect of rottlerin on glucose oxidation relative to glucose anaplerosis in the TCA cycle of MIA PaCa-2 cells.

FIG. 42 shows the effect of rottlerin on de novo myrystate (1), palmitate (2), stearate (3) and oleate (4) fatty acid synthesis of MIA PaCa-2 cells.

### DETAILED DESCRIPTION OF THE INVENTION

The present invention is directed to compounds, compositions, and methods for treating, preventing, and inhibiting cancer. Specifically, the present invention provides compositions comprising at least one plant-derived polyphenolic compound and at least one inhibitor of reactive oxygen species (ROS) to cause cancer cell death and prevent or treat cancer. The present invention also provides methods for treating or preventing cancer in a subject which comprises administering at least one plant-derived polyphenolic compound and at least one inhibitor of reactive oxygen species (ROS) to the subject.

Additionally, the present invention is directed to inducing apoptosis in cancer cells by modulating phosphatidylinositol 3-kinase Akt/PKB, generators of reactive oxygen species, nuclear factor-KB (NF-κB), mitochondrial permeability transition pore, mitochondrial polarity, mitochondrial cytochrome c release, caspases, or a combination thereof with at least one food-derived polyphenolic compound and at least one inhibitor of reactive oxygen species (ROS).

The invention described in the present application is designed to prevent and treat pancreatic and other cancers. The invention describes the use of combinations of prototype plant-derived polyphenolic compounds and inhibitors of reactive oxygen species to activate death pathways in the cancer cell. This effect on cell death pathways is specific to cancer cells so that normal tissue is unaffected while cancer cells in both primary tumor sites and metastatic sites die. The molecular targets affected by these combinations include phosphatidylinositol 3-kinase Akt/PKB, generators of reactive oxygen species, nuclear factor-KB, mitochondrial permeability transition pore, mitochondrial polarity, mitochondrial cytochrome c release, and caspases. The simultaneous effects of the combination of agents in this invention on these targets leads to cancer cell death. The result is slowing of the growth of the primary tumor as well as prevention of metastases. The invention can be used in strategies for both the prevention and treatment of pancreatic and other cancers.

A. Plant-Derived Polyphenolic Compounds

Evidence from population studies indicates a protective effect of fruits and vegetables in the diet of subjects on cancer. See Steinmetz & Potter (1991) Cancer Causes Control 2:325-357; and Norell et al. (1986) Am. J. Epidemiol. 124:894-902, which are herein incorporated by reference. Preliminary in vitro and animal experiments suggest that the polyphenolic phytochemical compounds in these foods may be involved in this beneficial effect. See Hsieh & Wu (1999); Huang et al. (1999); Islam et al. (2000); Sakagami et al. (2000); Gupta et al. (2000); Li et al. (2000); Paschka et al. (1998); Wang et al. (1999); Ahmad et al. (2000) Arch. Biochem. Biophys. 376:338-346; and Tsai et al. (1999) Br. J. Pharmacol. 126:673-680, which are herein incorporated by reference.

Although there are up to about 8000 plant polyphenolic compounds that have been identified, they can simply be divided into two groups: the flavonoids and the nonflavonoids. See Bravo (1998) Nutr. Rev. 56:317-333, which is herein incorporated by reference. Flavonoids are characterized as molecules possessing two phenols joined by a pyran (oxygen-containing) carbon ring structure. Common flavonoids include quercetin, rutin and genistein. Flavonoids represent the most common and widely distributed group of plant polyphenolic compounds. Examples of nonflavonoid polyphenolic compounds include the resveratrol family of compounds. Trans-resveratrol has recently received significant attention as a component in red wine and grapes that has anti-tumor and anti-inflammatory properties. See Jang et al. (1997) Science 275:218-220; and Subbaramaiah (1998) J. Biol. Chem. 273:21875-21882, which are herein incorporated by reference. The three polyphenolic compounds used in the Examples herein have the following structural formulas:

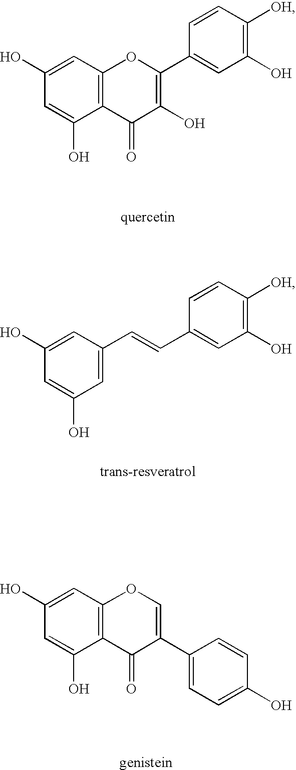

It should be noted, however, that use of these three polyphenolic compounds is exemplary only and that any polyphenolic compound may be readily used in accordance with the present invention. Polyphenolic compounds of the present invention include compounds that have more than one phenol ring structure. For example, flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid and traxol, and the like are plant polyphenolic compounds.

As provided in Example 1, quercetin treatment as tested in a nude mouse model of pancreatic cancer using the highly malignant pancreatic cancer cell line, Mia PACA-2, inhibited, prevented, or decreased metastatic cancer lesions. Quercetin treatment also significantly decreased the growth of the primary tumor. Therefore, the present invention provides a method of treating, preventing, or inhibiting cancer, preferably pancreatic cancer, in a subject, preferably human, comprising administering to the subject an effective amount of a polyphenolic compound. The present invention also provides a method of treating, inhibiting, preventing, or decreasing, metastatic cancer lesions in a subject, preferably human, comprising administering to the subject an effective amount of a polyphenolic compound. The present invention further provides a method of treating, inhibiting, or decreasing the growth or growth rate of a primary tumor in a subject, preferably human, comprising administering to the subject an effective amount of a polyphenolic compound. In some preferred embodiments, the polyphenolic compound is quercetin, trans-resveratrol, or genistein.

For the experiments described herein, incubation media free of serum was used in order to determine the effects of the agents in the absence of growth factors. When serum was subsequently added to the incubation conditions, the effects of the polyphenolic compounds described above were attenuated, thereby indicating that agents in serum have an effect on regulating the apoptosis pathways. As provided in Example 2, serum stimulates the formation of reactive oxygen species (ROS) in cancer cells. The presence of serum and insulin growth factor-1 (IGF-1) increases the formation of reactive oxygen species (ROS) in cancer cells, which is inhibited by antioxidants. Therefore, in preferred embodiments, the methods of the present invention further comprise administration of at least one antioxidant.

As provided in Example 3, quercetin, trans-resveratrol and genistein enhance apoptotic cancer cell death in pancreatic cancer cells by causing mitochondrial depolarization and cytochrome c release followed by caspase-3 activation. Inhibition of the mitochondrial PTP resulted in the prevention of mitochondrial depolarization, cytochrome c release, caspase-3 activation and apoptosis. Furthermore, both quercetin and genistein caused inhibition of growth of pancreatic cancer in a nude mouse model. The inhibition was most pronounced on metastatic spread of the tumor and included increased apoptosis in the tumor.

Therefore, the present invention provides a method of inducing apoptosis in a primary tumor comprising contacting the primary tumor with an effective amount of at least one polyphenolic compound. The present invention also provides a method of cleaving a protein target of caspases-3 activation, PARP, comprising contacting PARP with an effective amount of at least one polyphenolic compound.

Also, as described herein, the combination of quercetin and trans-resveratrol caused a synergistic increase in caspase-3 activity. Because quercetin is one of the most potent antioxidants of the polyphenolic compounds, the synergistic effects observed with the combination of quercetin and trans-resveratrol were likely due to the antioxidant effects of quercetin. Therefore, in preferred embodiments, the methods of the present invention further comprise administration of at least one antioxidant.

B. ROS Inhibitors

ROS are produced in large quantities by phagocytes mediating host defense against a variety of microorganisms. See Thannickal & Fanburg, (2000) Am. J. Physiol. Lung Cell. Mol. Physiol. 279:L1005-L1028; Freeman & Crapo (1982) Lab. Invest. 47: 412-426; Rhee (1999) Exp. Mol. Med. 31:53-59; and Babior (1999) Blood 93:1464-1476, which are herein incorporated by reference. There is accumulating evidence that ROS are produced in smaller quantities by non-phagocytes including cancer cells. See Nakamura et al. (1997) Ann. Rev. Immunol. 15:351-369 and Davis Jr., et al. (2001) J. Pharmacol. Exp. Ther. 296:1-6, which are herein incorporated by reference. At higher concentrations ROS have destructive effects on DNA, proteins and membranes. However, at low concentrations ROS are essential participants in regulation of cell proliferation and survival.

There are multiple potential sources of ROS. The best-characterized source is the NADPH oxidase system in phagocytes. Recent studies suggest that a group of functional proteins analogous to the NADPH oxidase system are present and mediate ROS in non-phagocytic cells. The proteins are called NOX proteins, which are homologous to the NADPH oxidase catalytic subunit, gp91phox. Another source of ROS generation is the mitochondria where ROS are produced as “by-products” of the electron transfer reactions. Other sources of ROS production include oxidation of the phospholipase A₂ product, arachidonic acid, by 5-lipoxygenases; and cytosolic xanthine oxidase. See Woo et al. (2000) Biochem. J. 348 Pt 3:525-530; and Goldman et al. (1997) Adv. Exp. Med. Biol. 407:289-293, which are herein incorporated by reference.

Although the source of the ROS is not subject of the current invention, it is important to emphasize their potential role and importance in cancer cell survival. First, human cancers produce ROS. See Szatrowski & Nathan (1991) Cancer Res. 51:794-798; Kong & Lillehei (1998) Med. Hypotheses 51:405-409; Thannickal et al. (2000) FASEB J. 14:1741-1748; and Bos (1989) Cancer Res. 49:4682-4689, which are herein incorporated by reference. Second, several cytokines and growth factors have been demonstrated to increase the production of ROS in cancer cells. These include TNFα, TGF-β, IL-1, interferon, PDGF and EGF. Available evidence suggests that the growth factors stimulate ROS by activating NADPH oxidase-like enzyme systems or phospholipase A₂ whereas the cytokines stimulate ROS production through mitochondrial mechanisms.

Furthermore, as provided herein, the addition of agents known to decrease the production of ROS caused a decrease in our measurement of cellular ROS using a fluorescent probe technique. For the Examples exemplified herein, three agents that attenuate ROS production were used. The first agent, diphenylene iodonium (DPI), which is a well-established inhibitor of NADPH oxidase, was used because of the likely possibility that the generator of ROS is a NADPH oxidase-like enzyme system. The other two agents were N-acetylcysteine (NAC) and Tiron, which are commonly used to “absorb” ROS.

Also, as provided herein, trans-resveratrol and genistein were found to cause a small increase in ROS production in addition to the effect of serum. Both the effects of serum and the polyphenolic compounds on ROS were prevented by DPI. Thus, serum, trans-resveratrol, and genistein increase ROS in pancreatic cancer cells and that agents known to inhibit ROS production prevent the increases in ROS.

As described herein, the effects of polyphenolic compounds alone and in combination with inhibitors of ROS formation on apoptosis of pancreatic cancer cells was studied. As provided herein, when cancer cells were treated with serum combinations of inhibitors of ROS and polyphenolic compounds resulted in a synergistic increases in cancer cell DNA fragmentation. On the other hand, the combination of DPI with a polyphenolic compound resulted in synergistic increases in DNA fragmentation. The effects of these agents on apoptosis were confirmed by Annexin V staining, which is another measure of apoptosis.

C. Caspases

In the recent past evidence has emerged for key roles for a family of cysteine proteases called caspases, the transcription factor, nuclear factor KB (NF-κB), and phosphatidylinositol 3-kinase (PI 3-kinase) in the mechanism of apoptosis. See Kromer & Reed (2000) Nature Medicine 6:513-519; Salvesen & Dixit (1997) Cell 91:443-446;

and Raff (1998) Nature 396:119-122; Green (1998) Cell 94:695-698; Wang et al. (1996) Science 274:784-787; Wang et al. (1999) Mol. Cell. Biol. 19:5923-5929; LaCasse et al. (1998); Kane et al. (1999) Curr. Biol. 9:601-604; Romashkova et al. (1999) Nature 401:86-90; Ozes et al. (1999) Nature 401:82-85; Madrid et al. (2000) Mol. Cell. Biol. 20:1626-1638; Xie et al. (2000) J. Biol. Chem. 275:24907-24914; and Madrid et al. (2001) J. Biol. Chem. 276:18934-18940, which are herein incorporated by reference.

Caspases are necessary for apoptosis to occur. More than a dozen caspases have been identified. The caspases are synthesized as inactive proenzymes requiring cleavage at Asp residues to be activated. At least some of these caspases can activate each other in the form of a proteolytic cascade. Caspases are generally divided into “initiator” caspases and “executioner” caspases. Caspases-8 and -9 are “initiator” caspases while caspases-3, -6 and -7 are “executioner” caspases.

Recent evidence indicates that there are two distinct pathways that mediate caspase activation and apoptosis. See Hsu et al. (1995) Cell 81:495-504; Hsu et al. (1996) Cell 84:299-308; Feinstein et al. (1995) Trends Biochem. Sci. 20:342-344; Green & Reed (1998) Science 281:1309-1312; and Yin et al. (1999) Nature 400:886-891, which are herein incorporated by reference. The first one involves the ligation of death receptors, i.e. TNFα R1, Fas, by their ligands resulting in the recruitment of adapter proteins, e.g. Fas activated death domain protein, FADD. See Feinstein et al. (1995) Trends Biochem. Sci. 20:342-344, which is herein incorporated by reference. The receptor-adapter protein complex, in turn, activates caspase-8. This caspase activates downstream “executioner” caspases such as caspase-3.

In the second pathway, various forms of cellular stress cause mitochondrial release of cytochrome c, which binds an adapter protein called APAFI along with ATP. See Green & Reed (1998) Science 281:1309-1312; Yin et al. (1999) Nature 400:886-891; and Crompton (1999) Biochem. J. 341:233-249, which are herein incorporated by reference. The resulting complex, in turn, binds and activates the “initiator” caspase-9. Caspase-9 then activates downstream “executioner” caspases, i.e. caspase-3. Although these two pathways are initially independent, they share activation of the downstream effector caspases. Furthermore, there is cross talk between the pathways. For example, caspase-8 cleaves a member of the Bc1-2 family, Bid. This protein then enhances mitochondrial cytochrome c release.

The mechanism of mitochondrial permeabilization and release of cytochrome c is incompletely understood. The permeabilization is usually associated with a loss of mitochondrial transmembrane potential and “opening” of the mitochondrial permeability transition pore (PTP). The PTP inhibitor, cyclosporine A, is frequently used to demonstrate the role of PTP in the involved in apoptosis, i.e. cytochrome c release and caspase activation.

As provided in Example 4, both quercetin and trans-resveratrol convert caspase-3 from its inactive form (32 kDa doublet) to its active form (17 kDa). Additionally, as described herein, caspase-3 activity is synergistically activated with a combination of an inhibitor of ROS production and a polyphenolic compound. Therefore, the present invention provides a method of activating caspase-3 comprising contacting the inactive caspase-3 with at least one ROS inhibitor or at least one polyphenolic compound. In preferred embodiments, the inactive caspase-3 is activated with at least one ROS inhibitor and at least one polyphenolic compound.

As provided in Example 5, quercetin, trans-resveratrol and genistein caused increases in cytosolic cytochrome c and decreases in mitochondrial cytochrome c. In particular, genistein stimulates both apoptosis and caspase-3 activation. Additionally, quercetin, trans-resveratrol, and genistein caused dissipation of mitochondrial membrane potential. Therefore, the present invention provides a method of increasing cytosolic cytochrome c, decreasing mitochondrial cytochrome c, dissipating mitochondrial membrane potential, or a combination thereof, comprising administering to a cell or a subject an effective amount of at least one polyphenolic compound.

D. NF-κB and Apoptosis

The mechanisms that mediate the potentiated effects of the polyphenols and inhibitors of ROS on apoptosis were also evaluated. Activated NF-κB may play a role in protecting cells from apoptosis. Many of the effects of NF-κB are thought to be mediated through a group of proteins called inhibitors of apoptosis (IAPs). See LaCasse et al. (1998) Oncogene 17:3247-3529, which is herein incorporated by reference. The effects of these mediators are mostly through the regulation of caspases. However, there are studies demonstrating that activated NF-κB can result in protection of mitochondria in cancer cells from dysfunction leading to cell death.

Insights into the regulatory role of NF-κB in apoptosis come from findings related to its activation induced by TNFα. See Wang et al. (1996) Science 274:784-787; Wang et al. (1999) Mol. Cell. Biol. 5923-5929; and LaCasse et al. (1998) Oncogene 3247-3529, which are herein incorporated by reference. TNFα stimulates apoptosis but the full extent of this stimulation in apoptosis is prevented by TNFα-induced activation of NF-κB. NF-κB activation occurs as a result of phosphorylation and degradation of NF-κB-associated proteins-IκBα and IκBβ (inhibitory κBs). Although the exact mechanism underlying the phosphorylation and degradation of the IκBs is incompletely understood, the phosphorylation can be initiated by IKK and phosphatidylinositol 3-kinase (PI 3-kinase) and modulated by ROS. See Kane et al. (1999) Curr. Biol. 9:601-604; Romashkova & Makarov (1999) Nature 401:86-90; Ozes et al. (1999) Nature 401:82-85; Madrid et al. (2000) Mol. Cell. Biol. 20:1626-1638; Xie et al. (2000) J. Biol. Chem. 275:24907-24914; Madrid et al. (2001) J. Biol. Chem. 276:18934-18940; Lepri et al. (2000) Cell. Biochem. Funct. 18:201-208; Lin et al. (1999) J. Biol. Chem. 274:13650-13655; McDade et al. (1999) J. Surg. Res. 83:56-61; Wolfet al. (2001) J. Biol. Chem. 276:34244-34251; Flohe et al. (1997) Free Radic. Biol. Med. 22:1115-1126, which are herein incorporated by reference. The phosphorylation of the IκB's leads to their transport to and rapid degradation by proteasomes. With IκB degradation, NF-κB translocates to the nucleus where it binds to promoter regions of target genes and activates them.

The mechanisms of the anti-apoptotic action of NF-κB are not fully understood. The known anti-apoptotic targets of activated NF-κB include the inhibitors of apoptosis (IAP) family of proteins, such as cIAP-1 and -2, and XIAP, as well as the anti-apoptotic Bc1-2 proteins.

As described herein, the effects of the polyphenolic compounds on mitochondrial dysfunction in pancreatic cancer cells incubated in the absence of serum of growth factors were independent from the effects on NF-κB activation. Since it was possible that in the presence of serum, at least one of the effects of the polyphenolic compounds could be through its effect on NF-κB. As provided in Example 7, the addition of serum to the incubation media caused an increase in NF-κB activation that was not inhibited by DPI, genistein, or trans-resveratrol alone, but was completely inhibited with the combination of DPI and either trans-resveratrol or genistein. The proteosome inhibitor, MG-132, blocks NF-κB activation in both cell lines and causes a small increase in caspase-3 activity. Additionally, trans-resveratrol in combination with MG-132 alone or MG-132 plus DPI increased apoptosis to a greater degree than that observed with MG-132 alone or MG-132 plus DPI, thereby indicating that inhibition of NF-κB sensitizes the cancer cells to apoptosis caused by trans-resveratrol.

Therefore, the present invention provides a method of preventing or inhibiting NF-κB activation in a cell comprising administering to the cell DPI and at least one polyphenolic compound or MG-132 and at least one polyphenolic compound or MG-132 and DPI. Since DPI is an antioxidant and MG-132 is a proteosomal inhibitor, the present invention provides a method of preventing or inhibiting NF-κB activation in a cell comprising administering to the cell an antioxidant, a proteosomal inhibitor, or both and at least one polyphenolic compound. The present invention also provides a method of making a cancer cell susceptible to apoptosis induced by a polyphenolic compound comprising inhibiting NF-κB activity in the cell.

E. Inhibition of NF-κB

In addition to inhibiting NF-κB activation in order to treat, prevent, or inhibit cancer as disclosed herein, U.S. Patent Application Publication No. 20040037902 published 26 Feb. 2004, which is herein incorporated by reference, describes the use of agents to inhibit activation of a transcription factor, nuclear factor-KB (NF-κB) for the treatment of pancreatitis as well as other inflammatory diseases.

Acute pancreatitis is a disorder the pathophysiology of which remains obscure. See Bhatia et al. (2000) J. Pathol. 190:117-125, Steer & Meldolesi (1987) N. Engl. J. Med. 316:144-150, Steer & Meldolesi (1988) Ann. Rev. Med. 39:94-105, and Steinberg & Tenner (1994) N. Engl. J. Med. 330:1198-1210, which are herein incorporated by reference. Although the complete mechanism of pancreatitis has not been established, there is a substantial body of evidence suggesting a critical role for the inflammatory response in this disease. See Grady et al. (1997) Gastroenterology 113:1966-1975, and Gukovsky et al. (1998) Am. J. Physiol. Gastrointest. Liver Physiol. 275:G1402-G1414, which are herein incorporated by reference. Research indicates that the initial events in this disorder occur in pancreatic acinar cells. More specifically, pancreatic acinar cells are capable of responding to noxious stimuli by upregulating signaling systems that mediate the production of proinflammatory mediators such as cytokines, chemokines, and adhesion molecules. See Gukovskaya et al. (1997) J. Clin. Invest. 100:1853-1862, Pandol et al. (1999) Gastroenterology 117:706-716, and Vaquero et al. (2001) Am. J. Physiol. Gastrointest. Liver Physiol. 280:G1197-G1208, which are herein incorporated by reference. These pancreas-generated mediators subsequently lead to the severe systemic complications of the disease.

A key regulator of the expression of these inflammatory molecules is NF-κB. See Schmid & Adler (2000) Gastroenterology 118:1208-1228, Tak & Firestein (2001) J. Clin. Invest. 107:7-11, Wulczyn et al. (1996) J. Mol. Med. 74:749-769, which are herein incorporated by reference. In experimental pancreatitis, NF-κB activation in acinar cells is one of the earliest events and the inhibition of NF-κB activation attenuates inflammatory response and the severity of pancreatitis. See Gukovsky et al. (2003) Am. J. Physiol. Gastrointest. Liver Physiol. 284:G85-G95, Satoh et al. (1999) Gut 44:253-258, and Steinle et al. (1999) Gastroenterology 116:420-430, which are herein incorporated by reference. Furthermore, the direct activation of NF-κB within the pancreas by adenoviral-mediated gene transfer is sufficient for the initiation of pancreatic and systemic inflammatory responses. See Chen et al. (2002) Gastroenterology 122:448-457, which is herein incorporated by reference.

Although previous studies demonstrated a key role for NF-κB activation in the mechanism of pancreatitis, the signaling mechanisms mediating NF-κB activation are unclear. Multiple factors are thought to contribute to induction of NF-κB activation in pancreatitis, and one important factor is TNF-α. See Algül et al. (2002) Am. J. Physiol. Gastrointest. Liver Physiol. 283:G270-G281, and Hietaranta et al. (2001) J. Biol. Chem. 276:18742-18747, which are herein incorporated by reference. In vivo, experiments on animal models demonstrated that TNF-α-induced NF-κB activation correlated with an increase in gene expression of various inflammatory molecules, and the administration of soluble TNF receptor or anti-TNF antibody prevented NF-κB activation in pancreatic acini and attenuated the inflammatory response and the severity of pancreatitis. See Grewal et al. (1994) Am. J. Surg. 167:214-218, Hughes et al. (1996) Am. J. Surg. 171:274-280, Norman et al. (1996) Surgery 120:515-521, which are herein incorporated by reference. Furthermore, the severity and mortality of pancreatitis was attenuated in mice deficient in TNF receptor. See Denham et al. (1997) Gastroenterology 113:1741-1746, which is herein incorporated by reference. TNF-α receptors are present in rat pancreatic acinar cells and TNF-α activates these receptors, thereby initiating signal transduction cascades including NF-κB activation. However, the post receptor events that link to NF-κB activation in pancreatic acinar cells are not established.

CCK-8 stimulation of isolated rat pancreatic acini can be also used to investigate the mechanism of NF-κB activation. See Han & Logsdon (1999) Am. J. Physiol. Cell. Physiol. 277:C74-C82, Han & Logsdon (2000) Am. J. Physiol. Cell. Physiol. 278:C344-C351, and Tando et al. (1999) Am. J. Physiol. Gastrointest. Liver Physiol. 277:G678-G686, which are herein incorporated by reference. CCK is a physiologic regulator of pancreatic digestive enzyme secretion; however, supramaximally stimulating doses of CCK-8 cause the inflammatory response that underlies many of the features of human pancreatitis. See Williams (2001) Ann. Rev. Physiol. 63:77-97, which is herein incorporated by reference. Similar to TNF-α, the post receptor events mediating NF-κB activation by CCK-8 are poorly understood.

One candidate for mediating NF-κB activation in pancreatic acinar cells is the family of protein kinase Cs (PKCs) because the incubation of pancreatic acinar cells with phorbol esters, a general activator of PKCs, causes NF-κB activation. See Gukovskaya et al. (2004) Am. J. Physiol. Gastrointest. Liver Physiol. 286:G204-G213, which is herein incorporated by reference. PKCs are a family of serine/threonine kinases comprising 10 isoforms that differ in their structures and regulations. See Dempsey et al. (2000) Am. J. Physiol. Lung Cell Mol. Physiol. 279:L426-L438, and Ron & Kazanietz (1999) FASEB J 13:1658-1676, which are herein incorporated by reference. These isoforms are subdivided into three classes on the basis of their molecular structure and mode of activation, namely, conventional PKC isoforms (α, βI, βII, and γ), novel PKC isoforms (δ, ε, η, and θ), and atypical PKC isoforms (ζ and λ/τ). The conventional PKC isoforms are activated by Ca²⁺ and by diacylglycerol (DAG) or phorbol esters. Of note, CCK stimulates increases in Ca²⁺ and DAG in pancreatic acinar cells through phospholipase C (PLC) pathway. The novel PKC isoforms are also activated by DAG and phorbol esters but are Ca²⁺ independent. The atypical PKC isoforms are unresponsive to Ca²⁺, DAG, and phorbol esters.

In addition to the regulation by Ca²⁺ and lipid messengers, the activity of PKCs is regulated by phosphorylation and one important mediator of this pathway is the family of Src kinases. See Gschwendt (1999) Eur. J. Biochem. 259:555-564, and Parekh et al. (2000) EMBO J. 19:496-503, which are herein incorporated by reference. Each PKC isoform has a different pattern of cell distribution, can be activated independently by specific stimuli, and mediates distinct biological functions. In general, the activation of PKCs is associated with their translocation to distinct intracellular compartments, and specific anchoring proteins target individual PKCs to different intracellular components and confer specificity for different substrates. See Mochly-Rosen & Gordon (1998) FASEB J. 12:35-42, and Gschwendt et al. (1996) FEBS Lett. 392:77-80, which are herein incorporated by reference.

As provided herein, the signaling pathways mediating NF-κB activation in pancreatic acinar cells induced by high-dose of CCK-8 (which causes pancreatitis in subjects) and TNF-α (which contributes to inflammatory responses), especially, the role of PKC isoforms, was studied. Subcellular distribution and kinase activities of PKC isoforms, and NF-κB activation in dispersed rat pancreatic acini were examined. Isoform-specific, cell-permeable peptide inhibitors were used to assess the role of individual PKC isoforms in NF-κB activation.

Both CCK-8 and TNF-α activated the novel isoforms, PKC δ and E, and the atypical isoform, PKC ζ, but not the conventional isoform, PKC α. Inhibition of the novel PKC isoforms, but not the conventional or the atypical isoforms, resulted in the prevention of NF-κB activation induced by CCK-8 and TNF-α. NF-κB activation by CCK-8 and TNF-α required translocation but not tyrosine phosphorylation of PKC 6. The activation of PKC δ, PKC ε, and NF-κB with CCK-8 involved both phosphatidylinositol-specific PLC and phosphatidylcholine (PC)-specific PLC, whereas with TNF-α they only required PC— specific PLC for activation. These results indicate that CCK-8 and TNF-α initiate NF-κB activation by different PLC pathways, which converge at the novel PKCs, δ and ε, to mediate NF-κB activation in pancreatic acinar cells.

As provided herein, PKC δ and ε are responsible for both CCK-8-induced and TNF-α-induced NF-κB activation in pancreatic acinar cells. Translocation but not phosphorylation of PKC δ is necessary for mediating NF-κB activation. Pharmacologic analysis showed that both phosphatidylinositol (PI)-specific PLC and phosphatidylcholine (PC)-specific PLC are necessary for the activation of PKC δ, PKC ε, and NF-κB by CCK-8. In contrast, these responses occur only through PC-specific PLC in acini stimulated with TNF-α. Although CCK-8 and TNF-α initiate NF-κB activation by different PLC pathways, these pathways converge on the activation of PKC δ and PKC ε, leading to NF-κB activation in pancreatic acinar cells.

As provided herein, peptides were found to block the translocation of either PKCδ or PKCε, thereby inhibiting activation of NF-κB in pancreatic acinar cells. Thus, these peptide inhibitors and other agents that block the translocation of one or both of these PKC isoforms will result in inhibition of activation of NF-κB may be used to treat, prevent, or inhibit pancreatitis and other diseases in which NF-κB is involved in the pathogensesis such as inflammation, cancer, and the like.

As used herein, the phrase “diseases and disorders associated with NF-κB activation” is used interchangeably with the phrase “diseases and disorder in which NF-κB is involved in the pathogensesis”. Diseases and disorders associated with NF-κB activation include proliferative diseases such as cancer and inflammatory diseases such as pancreatitis.

Cancers associated with NF-κB activation include breast cancer, ovarian cancer, prostate cancer, kidney cancer, pancreatic cancer, colon cancer, thyroid cancer, melanoma, Hodgkin's lymphoma, acute lymphoblastic leukemia, acute myelogenous leukemia, diffuse large B-cell lymphoma, astrocytoma, glioblastoma, head and neck cancers, vulva cancer, and the like. See Gilmore et al. (2002) Cancer Lett. 181:1-9, Bargou et al. (1997) J. Clin. Invest. 100:2961-2969, Huang et al. (2001) Oncogene 20:4188-4197, Huang et al. (2000) Cancer Res. 60:5334-5339, Nakshatri et al. (1997) Mol. Cell. Biol. 17:3629-3639, Sovak et al. (1997) J. Clin. Invest. 100:2952-2960, Dejardin et al. (1999) Oncogene 18:2567-2577, Pajonk et al. (1999) J. Nat'l Cancer Inst. 91:1956-1960, Palayoor et al. (1999) Oncogene 18:7389-7394, Oya et al. (2001) Oncogene 20:3888-3896, Tai et al. (2000) Cancer 89:2274-2281, Wang et al. (1999) Clin. Cancer Res. 5:119-127, Lind et al. (2001) Surgery 130:363-369, Visconti et al. (1997) Oncogene 15:1987-1994, Ludwig et al. (2001) Cancer Res. 61:4526-4535, Meyskens Jr. et al. (1999) Clin. Cancer Res. 5:1197-1202, Yang et al. (2001) Cancer Res. 61:4901-4909, Kordes et al. (2000) Leukemia 14:399-402, Dokter et al. (1995) Leukemia 9:425-432, Guzman et al. (2001) Blood 98:2301-2307, Hayashi et al. (2001) Neurol. Med. Chir. 41:187-195, Ondrey et al. (1999) Mol. Carinog. 26:119-129, Tamatani et al. (2001) Cancer Lett. 171:165-172, and Seppanen et al. (2000) Immunol. Lett. 74:103-109, which are herein incorporated by reference. Cancers associated with NF-κB activation also include cancers caused by in vitro transformation including BCR-ABL, DBL/DBS, RAF, RAS, TEL-JAK2, TEL-PDGFR, and the like, and viral oncogenesis caused by Epstein-Barr virus, hepatitis B virus, human herpesvirus-8, and human T-cell leukemia virus-1, and the like. See Gilmore et al. (2002) Cancer Lett. 181:1-9, Jeang (2001) Cytokine Growth Factor Rev. 12:207-217, Cahir et al. (1999) Oncogene 18:6959-6964, Reuther et al. (1998) Genes Dev. 12:968-981, Whitehead et al. (1999) Mol. Cell. Biol. 19:7759-7770, Baumann et al. (2000) PNAS USA 97:4615-4620. Finco et al. (1997) J. Biol. Chem. 272:24113-24116, Santos et al. (2001) FEBS Lett. 497:148-152, Besancon et al. (1998) PNAS USA 95:8081-8086, Diao et al. (2001) Cytokine Growth Factor Rev. 12:189-205, and Pati et al. (2001) J. Virol. 75:8660-8673, which are herein incorporated by reference.

Inflammatory diseases associated with NF-κB activation include inflammatory bowel disease, asthma, arthritis including rheumatoid arthritis, asthma, psoriasis, cystitis, nephritis, and the like. See Barnes & Karin (1997) N. Engl. J. Med. 336(15):1066-1071, Abdel-Mageed (2003) Urol. Res. 31(5):300-305, and Lopez-Franco et al. (2002) Am. J. Pathol. 161(4):1497-1505, which are herein incorporated by reference.

Other diseases and disorders associated with NF-κB activation include viral hepatitis, alcoholic liver disease, lung inflammation, Alzheimer's Disease, Atherosclerosis, and the like. See Waris & Siddiqui (2003) J. Biosci. 28(3):311-321, Hirano et al. (2003) J. Hepatol. 38(4):483-489, Haeberle et al. (2004) J. Virol. 75(5):2232-2241, Gao et al. (2002) Mol. Brain Res. 105(1-2):108-114, Martin-Ventura et al. (2004) Stroke 35(2):458-463, and Verma et al. (2003) J. Thorac. Cardiovasc. Surg. 126(6):1886-1891, which are herein incorporated by reference.

As disclosed herein, in order to test whether inhibitors of translocation of PKCδ and PKCε affect activation of NF-κB by CCK and TNF-α, the following inhibitors were synthesized using methods known in the art:

a PKC δ translocation inhibitor

| | |
|---|---|
| δV1-1:SFNSYELGSL | (SEQ ID NO:1) |

a PKC ε translocation inhibitor

| | |
|---|---|
| εV1-2:EAVSLKPT | (SEQ ID NO:2) |

and control peptide

| | |
|---|---|
| LSETKPAV | (SEQ ID NO:3) |

according to previous studies. See Chen et al. (2001) PNAS USA 98:11114-11119, and Dorn et al. (1999) PNAS USA 96:12798-12803, which are herein incorporated by reference. These peptides correspond to the specific sequences in the V1 regions of each of the PKC isoforms. The V1 region is responsible for anchoring the specific PKC to its translocation site. Thus, these peptides competitively inhibit the binding of a specific isoform of PKC to its anchoring protein. These peptides were then conjugated to the following Drosophila antennapedia peptide using methods known in the art:

| | |
|---|---|
| RQIKIWFQNRRMKWKK | (SEQ ID NO:4) |

to make them cell-permeable.

CCK-8 causes a rapid and prolonged NF-κB activation in pancreatic acinar cells in a dose and time dependent manner, and that the response of NF-κB to 100 nM CCK-8 reaches a maximum at 30 minutes after the stimulation. See Gukovsky (1998) Am. J. Physiol. 275:G1402-G1414, and Pandol et al. (1999) Gastroenterology 117:706-716, which are herein incorporated by reference. Based on these results, pancreatic acini prepared from normal rats were preincubated with each PKC translocation inhibitor (10 μM) or same volume of DMSO for 3 hours, and then stimulated with CCK-8 (100 nM) or TNF-α (100 ng/ml) for 30 minutes in the series of experiments provided in the Examples. Control peptide (10 μM) instead of DMSO was used as the control for the translocation inhibitors. At the end of this period, the effects of the treatments on activation of NF-κB as well as degradation of inhibitory-KB (1-KB), another measure indicating activation of NF-κB, were measured.

As provided herein, the cell permeant peptide translocation inhibitors specifically blocked translocation of the isoform of PKC they were intended to inhibit. Furthermore, both inhibitors prevented NF-κB activation. This inhibition in activation of NF-κB may be used as provided in Section D above as well as the attenuation of inflammatory responses and pancreatitis as disclosed in U.S. Patent Application Publication No. 20040037902 published 26 Feb. 2004, which is herein incorporated by reference.

1. CCK-8 Activates PKC δ, ε, and ζ, but not PKC α, in Rat Pancreatic Acini

The presence and translocation of each PKC isoform was assayed by Western blot methods known in the art. As previously reported in the art and as shown in FIG. 19A, immunoreactivities to four isoforms of PKC (α, δ, ε, and ζ) were detected in untreated rat pancreatic acini, with a large percentage of each isoform residing in the cytosolic fraction. Treatment with 100 nM CCK-8 decreased the presence of PKC δ and PKC ε in the cytosolic fraction and increased them in the membrane fraction, thereby indicating translocation from cytosol to cell membranes. In contrast, no changes in the subcellular localization of PKC α or PKC ζ were detected after CCK-8 stimulation. See FIG. 19A.

Kinase assays using PKC isoform specific immunoprecipitates were then conducted. The CCK-8 treatment increased kinase activities for PKC δ, PKC ε, and PKC ζ as shown in FIG. 19B. At the same time, PKC α activity was not significantly altered by CCK-8. These data show the distinct responses of PKC isoforms: CCK-8 stimulated both kinase activity and translocation of PKC δ and PKC E; it increased PKC 4 activity without affecting its translocation; and it had no effect on kinase activity and translocation of PKC α.

2. Inhibition of PKC δ and ε Prevents CCK-8-Induced NF-κB Activation

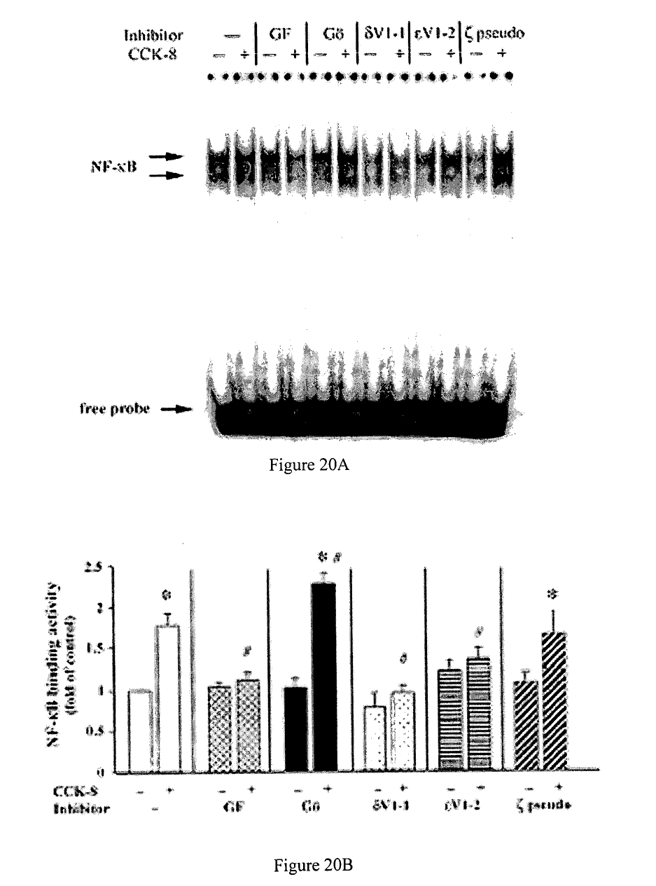

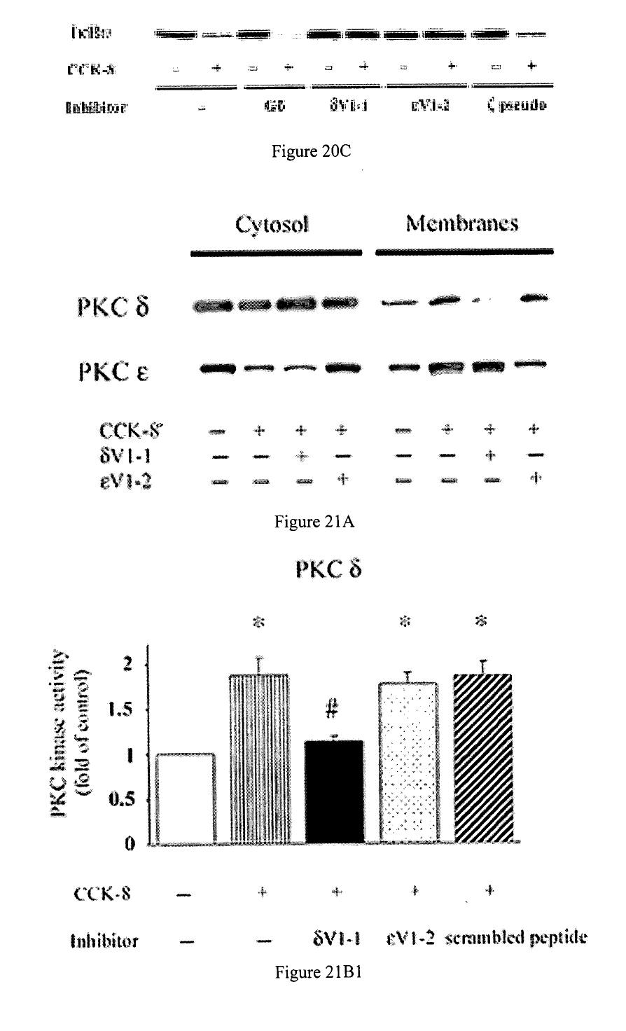

To determine the PKC isoform(s) that mediate NF-κB activation by CCK-8, pharmacologic analysis with isoform specific PKC inhibitors was preformed using methods known in the art. The activation of NF-κB was determined by NF-κB binding activity and IκBα degradation. The CCK-8 induced NF-κB activation was inhibited by the broad spectrum PKC inhibitor, GF109203X, the PKC δ translocation inhibitor, δV1-1, and the PKC ε translocation inhibitor, εV1-2, by about 98%, about 76%, and about 80%, respectively, as shown in FIG. 20A and FIG. 20B. The conventional PKC isoform inhibitor, Gö6976, did not inhibit, but rather enhanced the NF-κB response. See FIG. 20A and FIG. 20B. PKC ζ pseudosubstrate did not affect NF-κB activation as shown in FIG. 20A and FIG. 20B while abolishing the increase in kinase activity of PKC ζ (data not shown). None of the inhibitors alone affected the basal NF-κB activity. See FIG. 20B. The degradation of IκBα correlated with the increased NF-κB binding activity in CCK-8 treated cells, and the blockade of IκBα degradation by δV1-1 or εV1-2 was consistent with their inhibitory effects on NF-κB binding activity. See FIGS. 20A-20C. Gö6976 enhanced CCK-8 induced IκBα degradation as shown in FIG. 20C. These results indicate that PKC δ and PKC δ are responsible for CCK-8-induced NF-κB activation in pancreatic acinar cells. In contrast, the data suggest that PKC α may exert an inhibitory effect on the NF-κB activation.

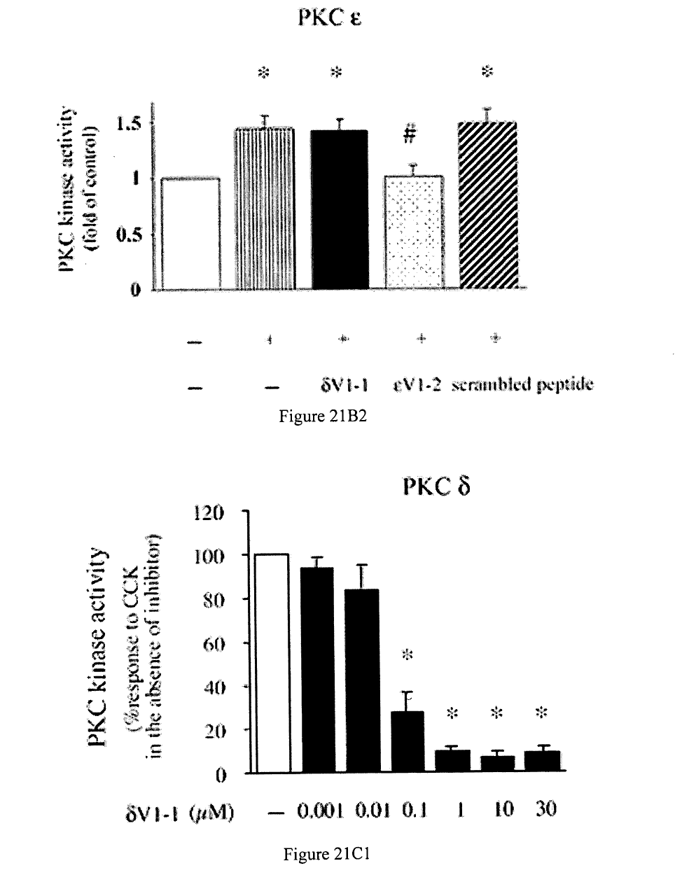

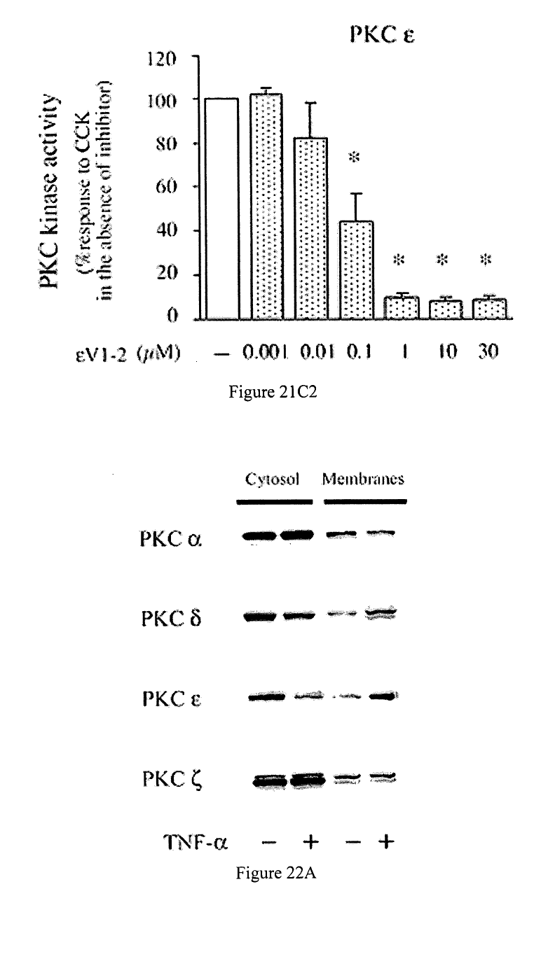

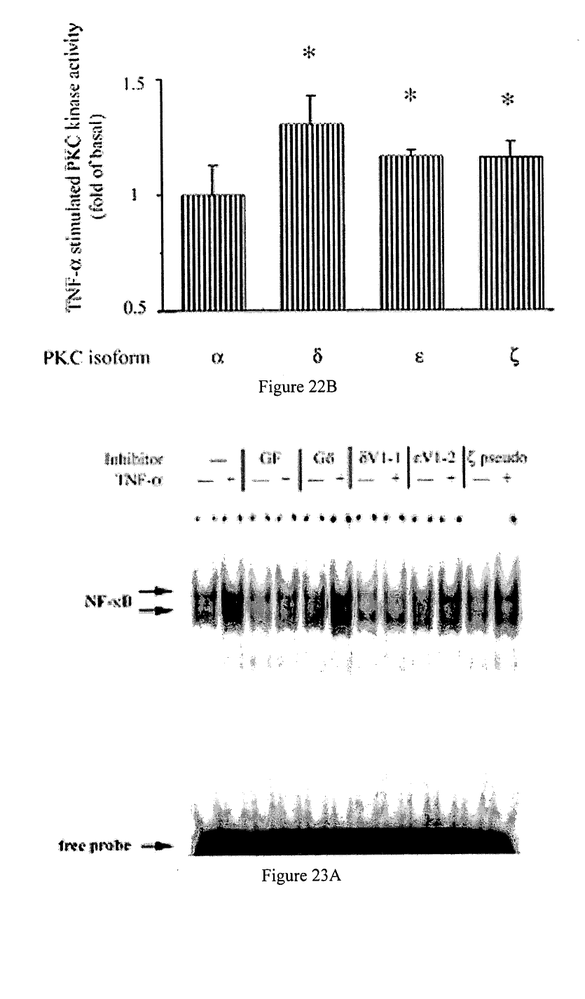

To demonstrate the specificity of the translocation inhibitors, δV1-1 and εV1-2, in pancreatic acini, their effects on PKC δ and PKC ε was examined. As shown in FIG. 21A, δV1-1 prevented the translocation of PKC δ but not that of PKC ε, whereas εV1-2 blocked the translocation of PKC ε without affecting PKC δ. Furthermore, each inhibitor abolished the increase in their target isoform's kinase activity without affecting the other isoform's activity as shown in FIG. 21B. The scrambled peptide did not affect the translocation and kinase activity of either isoform. The fact that neither δV1-1 nor εV1-2 inhibited kinase activity of the PKCs when applied directly into the assay was confirmed (data not shown).

Therefore, the present invention provides methods of inhibiting NF-κB activation comprising preventing PKC δ translocation, PKC ε translocation, or both. In some embodiments, preventing PKC δ translocation comprises contacting δV1-1 with PKC 6. In some embodiments, preventing PKC ε translocation comprises contacting εV1-2 with PKC ε. The present invention also provides methods of treating, preventing, or inhibiting a disease or disorder associated with NF-κB activation, such as abnormal cell proliferation, e.g. cancer, and inflammation, e.g. pancreatitis, and the like, which comprises inhibiting NF-κB activation. In some embodiments NF-κB activation is inhibited by preventing PKC δ translocation, PKC ε translocation, or both.

3. TNF-α Activates PKC δ, ε, and ζ, but not PKC α, in Rat Pancreatic Acini

Inhibition of PKC δ and ε Prevents TNF-α Induced NF-κB Activation

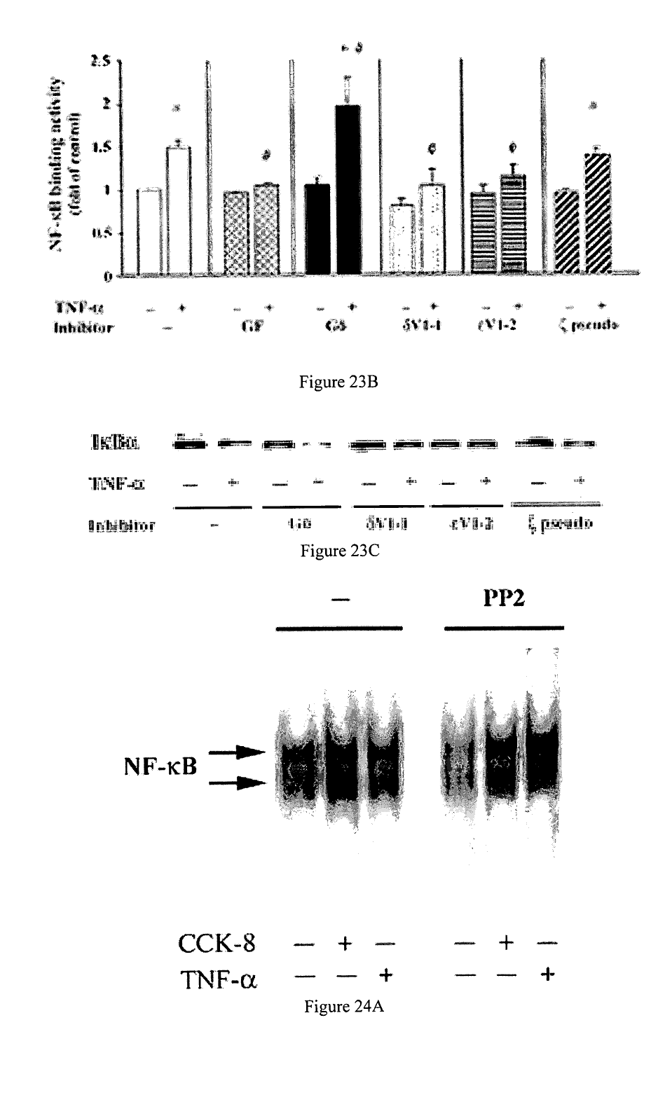

As provided herein, pancreatic acini were stimulated with 100 ng/ml TNF-α. Similar to CCK-8, TNF-α induced translocation of PKC δ and ε, but not α or ζ as shown in FIG. 22A. In cells stimulated by TNF-α, increases in kinase activity were observed in PKC δ, ε, and ζ, but not in α as shown in FIG. 22B. As shown in FIG. 23, TNF-α caused NF-κB activation in pancreatic acini. Compared to CCK-8, the responses of both PKC and NF-κB to TNF-α were relatively smaller, but see FIG. 20B and FIG. 23B. When acini were pretreated with GF109203X, the increase in NF-κB binding activity by TNF-α was abolished, indicated participation of PKC isoforms in the NF-κB activation by TNF-α. The TNF-α induced NF-κB activation was inhibited by GF109203X, δV1-1, and εV1-2, by 81%, 57%, and 58%, respectively. See FIG. 23A and FIG. 23B. The conventional PKC isoform inhibitor, Gö6976, did not inhibit, but rather enhanced the NF-κB response. PKC ζ pseudosubstrate did not affect NF-κB activation. See FIG. 23A and FIG. 23B. Consistent with the results of NF-κB binding activity, TNF-αα-induced degradation of IκBα was blocked by δV1-1 and εV1-2, enhanced by Gö6976, and unaffected by PKC ζ pseudosubstrate as shown in FIG. 23C. These results indicate that PKC δ and PKC ε are responsible for TNF-α-induced NF-κB activation and that PKC α may exert an inhibitory effect on the NF-κB activation.

Therefore, the present invention provides methods of inhibiting, preventing, or modulating TNF-α induced NF-κB activation in a cell or a subject which comprises administering to the cell or the subject GF109203X, δV1-1, εV1-2, or a combination thereof.

4. Src Kinase Inhibitor Does Not Prevent CCK-8- or TNF-α-Induced NF-κB Activation

In pancreatic acinar cells, Src kinases have been implicated as upstream modulators of PKC in response to CCK-8. See Ferris et al. (1999) Biochemistry 38:1497-1508, Tsunoda et al. (1996) Biochem. Biophys. Res. Commun. 227:876-884, Tapia et al. (2002) Biochim. Biophys. Acta. 1593:99-113, and Tapia et al. (2003) J. Biol. Chem. 278:35220-35230, which are herein incorporated by reference. Src kinases have also been linked to NF-κB activation in a number of cell types. See Abu-Amer et al. (1998) J. Biol. Chem. 273:29417-29423, Devary et al. (1993) Science 261:1442-1445, and Li et al. (1998) PNAS USA 95:5718-5723, which are herein incorporated by reference.

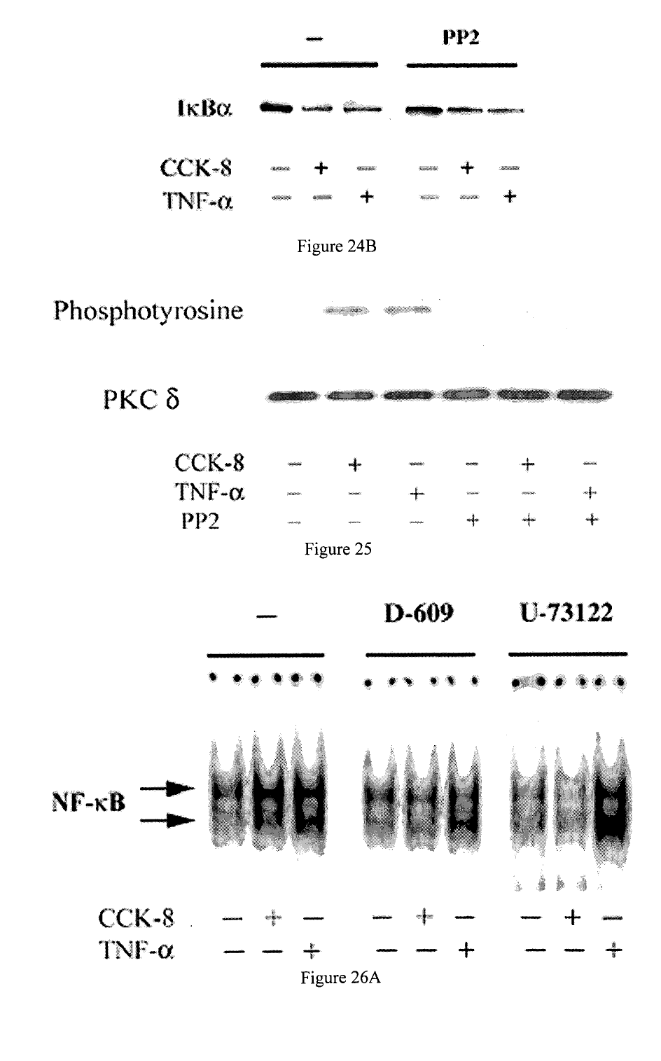

To investigate whether Src tyrosine kinases are involved in the activation of NF-κB in pancreatic acinar cells, PP2, a specific inhibitor of Src kinases was applied. Pretreatment of pancreatic acini with PP2 inhibited neither NF-κB binding activity nor IκBα degradation induced by CCK-8 as shown in FIG. 24A and FIG. 24B. Similarly, PP2 had no effect on NF-κB activation induced by TNF-α. See FIG. 24A and FIG. 24B. PP1, another Src kinase inhibitor, also did not prevent CCK-8-induced or TNF-α-induced NF-κB activation (data not shown).

Another regulatory pathway for PKC activation is tyrosine phosphorylation. See Konishi et al. (1997) PNAS USA 95:11233-11237, which is herein incorporated by reference. Among the PKC family, PKC δ is the most efficiently tyrosine phosphorylated isoform. As provided herein, PP2 almost completely inhibited tyrosine phosphorylation of PKC δ induced by CCK-8 and TNF-α as shown in FIG. 25. These results indicate that Src kinases mediate tyrosine phosphorylation of PKC δ but are not involved in NF-κB activation induced by CCK-8 and TNF-α in pancreatic acini. Thus, CCK-8 and TNF-α induced tyrosine phosphorylation of PKC δ and pretreatment of pancreatic acini with PP2, a specific inhibitor of Src kinases, abolished the tyrosine phosphorylation of PKC δ induced by CCK-8 and TNF-α.

5. CCK-8 Activates the Novel PKC Isoforms and NF-κB Through Both PI-Specific PLC and PC-Specific PLC, Whereas TNF-α Activates Them Through Only PC-Specific PLC

Previous studies demonstrated that CCK-8 activates PKC through activation of PLC, which results in the hydrolysis of phosphatidylinositol as well as phosphatidylcholine, resulting in the production of DAG. See Matozaki & Williams (1989) J. Biol. Chem. 264:14729-14734, and Sarri et al. (2000) FEBS Lett. 486:63-67, which are herein incorporated by reference.

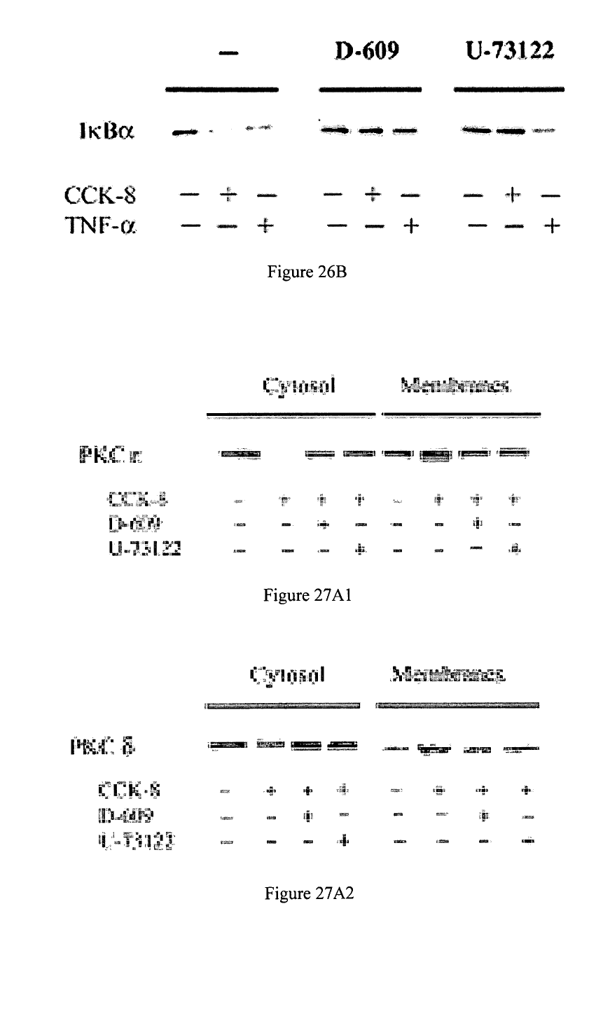

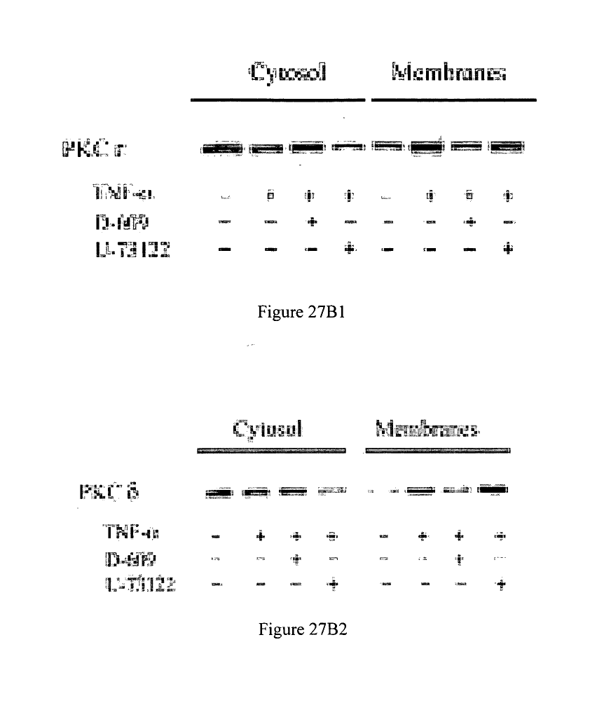

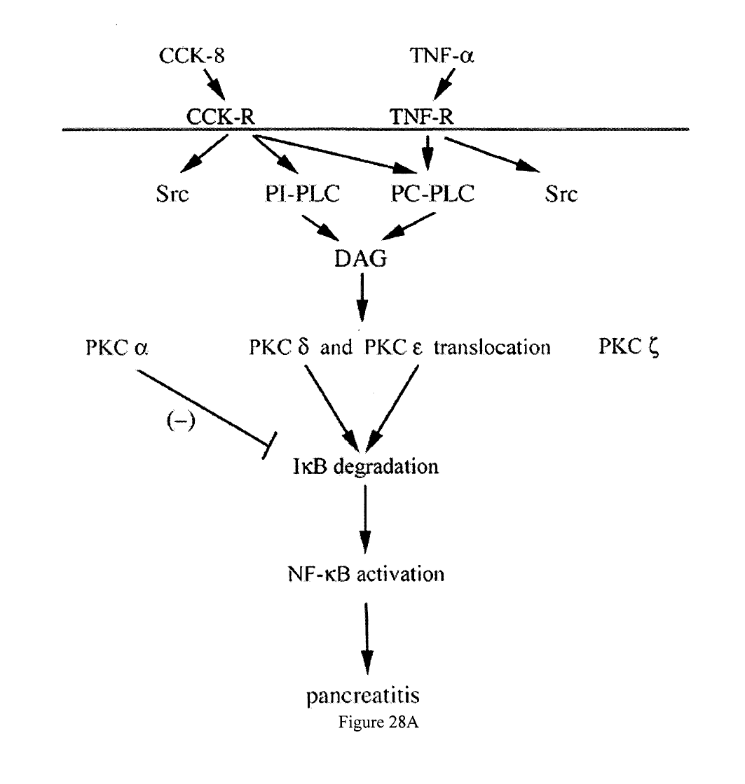

To investigate whether the activation of PLC is involved in the NF-κB activation in pancreatic acini, U-73122, an inhibitor of PI-specific PLC inhibitor, and D-609, a PC-specific PLC inhibitor, were applied. In D-609 treated cells, NF-κB activation following stimulation with CCK-8 or TNF-α was significantly attenuated as shown in FIG. 26A and FIG. 26B. U-73122 prevented the NF-κB activation induced by CCK-8 but not that by TNF-α. See FIG. 26A and FIG. 26B. U-73122 did not affect the TNF-α-induced NF-κB activation at concentrations up to 30 μM (data not shown). As shown in FIG. 27, the CCK-8-induced translocation of PKC δ and ε was prevented by both U-73122 and D-609. In contrast, only D-609 inhibited the translocation of PKC 8 and ε induced by TNF-α. These results indicate that the activation of NF-κB and the novel PKCs by CCK-8 is regulated through both PI-specific PLC and PC-specific PLC, whereas TNF-α-induced responses only involve PC-specific PLC pathway.

Both phosphatidylinositol and phosphatidylcholine are the main precursors of DAG generation in pancreatic acinar cells after CCK stimulation. See Hermans et al. (1996) Eur. J. Biochem. 235:73-81, Pandol & Schoeffield (1986) J. Biol. Chem. 261:4438-4444, Pandol. Et al. (1985) Am. J. Physiol. Gastrointest. Liver Physiol. 248:G551-G560, which are herein incorporated by reference. On the other hand, TNF-α activated the novel PKCs and NF-κB in pancreatic acini through only PC-specific PLC. The activation of PC-specific PLC by TNF-α and the subsequent activation of NF-κB have been shown in a number of cell types and the TNF receptor 1 is associated with the process. See Adam et al. (1996) J. Biol. Chem. 271:14617-14622, Machleidt et al. (1996) J. Exp. Med. 184:725-733, Plo et al. (2000) Biochem. J. 351(2):459-467, Schutze et al. (1991) J. Exp. Med. 174:975-988, Schütze et al. (1994) J. Leukoc. Biol. 56:533-541, and Schütze et al. (1992) Cell 71:765-776, which are herein incorporated by reference. Importantly, the TNF receptor 1 has been shown to mediate the inflammatory response in pancreatitis and is functional on pancreatic acinar cells. Considering these findings, TNF-α may accelerate the inflammatory response by producing DAG through PC-specific PLC. In turn, DAG may mediate NF-κB activation by promoting translocation of PKC δ and PKC ε.

In conclusion, the results herein, demonstrate the distinct responses of the PKC isoforms. CCK-8 and TNF-α increase both the kinase activity of PKC δ and ε and their translocation from the cytosolic to membrane fractions. Both stimuli increased the kinase activity of PKC 4 while the effect on its translocation was not apparent. It is possible that translocation of PKC ζ at a very weak level is involved in the responses or that CCK-8 and TNF-α activate PKC ζ without its translocation. On the other hand, there were no increases in either the kinase activity or the translocation of PKC α with both CCK-8 and TNF-α. This result is consistent with previous studies in which CCK-8 did not cause translocation of PKC α.

As provided herein, the translocation inhibitor peptides, δV1-1 and εV1-2, designed to competitively inhibit the binding of PKC δ and PKC ε to specific anchoring proteins, prevented the increases in kinase activity and translocation of their target PKC isoforms. These results indicate that translocation of PKC δ or PKC ε is involved in activation by CCK-8 and TNF-α in pancreatic acini. Of note, δV1-1 and εV1-2 did not cross-inhibit the PKC isoforms, indicating the high specificity of these peptide inhibitors. Prior to the present invention, these peptide inhibitors have not been previously applied to study the role of PKC in pancreatic acinar cells.

PKC δ and ε translocation inhibitors prevented both the CCK-8-induced and TNF-α-induced NF-κB activation, determined by NF-κB binding activity and IκBα degradation. These results indicate that PKC δ and PKC E are key mediators of the NF-κB activation in pancreatic acinar cells. Because each isoform-specific inhibitor prevented NF-κB activation to about the same degree without affecting the kinase activity and localization of the other PKC isoform, PKC δ and E regulate NF-κB activation independently at the level of IκBα degradation or upstream. In contrast, neither the inhibitor of conventional PKC isoforms nor the PKC ζ inhibitor prevented the NF-κB activation. Of note, the conventional PKC isoform inhibitor, Gö6976, augmented the NF-κB activation in response to both CCK-8 and TNF-α. These results indicate that constitutive activity of PKC α may have an inhibitory effect on NF-κB activation.

The Src kinase inhibitor, PP2, did not prevent the NF-κB activation by CCK-8 and TNF-α while completely inhibiting the tyrosine phosphorylation of PKC δ. Thus, it seems likely that neither the activation of Src kinases nor tyrosine phosphorylation of PKC δ is required for NF-κB activation. Tyrosine kinase inhibitors, including PP2, block the increase in kinase activity of PKC δ stimulated by CCK-8 in pancreatic acini while having no or little effect on translocation of PKC 6. In conjunction with these data, the results herein suggest that the NF-κB activation depends primarily on the translocation of PKC δ but not on kinase activity of PKC 6.

In summary, the results herein, indicate that translocation of novel PKC isoforms, PKC δ and PKC ε, is necessary for both CCK-8-induced and TNF-α-induced NF-κB activation. These results show that Src kinases regulate tyrosine phosphorylation of PKC δ, but they do not mediate NF-κB activation induced by CCK-8 or TNF-α. Thus, tyrosine phosphorylation of PKC δ is not involved in the NF-κB activation. The activation of PKC δ, PKC ε, and NF-κB with CCK-8 requires both PI-specific PLC and PC-specific PLC pathways, whereas with TNF-α they require only PC-specific PLC for activation.

F. PI 3-Kinase

Next, the possibility that phosphatidylinositol 3-kinase (PI 3-kinase) and Akt/PKB mediate the effects of serum on NF-κB; and that the effects of the polyphenols on NF-κB activation are due, at least in part, to an ability to inhibit PI 3-kinase were studied. The PI 3-kinase signaling system was used because it is an important mediator of responses to growth factors and because there is evidence that polyphenolic compounds such as quercetin and genistein inhibit PI 3-kinase and/or Akt/PKB. Of note, one commonly used inhibitor of PI 3-kinase is LY294002 (Calbiochem, San Diego, Calif.), a derivative of quercetin. Also, there are some suggestions for a role of ROS in activation of PI 3-kinase. Finally, and most importantly, there are several publications indicating that one of the effects of PI 3-kinase signaling is the activation of NF-κB. See Kane et al. (1999) Nature 401:86-99; Ozes et al. (1999) Nature 401:82-85; Madrid et al. (2000) Mol. Cell. Biol. 20:1626-1638; Xie et al. (2000) J. Biol. Chem. 275: 24907-24914; and Madrid et al. (2001) J. Biol. Chem. 276:18934-18940, which are herein incorporated by reference.

PI 3-kinase is an important signaling system that is activated by growth factors and G protein-coupled receptors that have been determined to regulate various cellular processes including proliferation, survival, inflammation and metabolism. See Katada et al. (1999) Chem. Phys. Lipids 98:79-86; and Leevers et al. (1999) Curr. Opin. Cell. Biol. 11:219-225, which are herein incorporated by reference. The activation of PI 3-kinase results in an increase in D-3 phosphorylated phosphoinositides such as phosphatidylinositol-3 phosphate, phosphatidylinositol-3,4 bisphosphate and phosphatidylinositol-3,4,5 trisphosphate.

The PI 3-kinase that is stimulated by tyrosine kinase activating receptors is relevant to the present application. This PI 3-kinase is structurally characterized as a heterodimer consisting of a 110-kD catalytic subunit (p110-α, β or -γ) and an 85-kD regulatory subunit (p85). Stimulation of tyrosine kinase activating receptors by extracellular signals, i.e. insulin and insulin-related growth factors results in phosphorylation of the receptor or receptor associated adapter proteins. The phosphorylated receptor or adapter proteins then bind to regulatory p85, which, in turn, activates catalytic p110. Of particular note, although structurally different, there is no difference in function between the isotypes of p110 described to date. Furthermore, each isotype is inhibited specifically by wortmannin and LY294002. LY294002 acts on an ATP-binding site while wortmannin blocks the catalytic activity of PI 3-kinase. See Balla (2001) Curr. Pharm. Des. 7:475-507, which is herein incorporated by reference.

As indicated above, with activation of PI 3-kinase, there is formation of D-3 phosphorylated phosphoinositides. These phospholipids then activate a protein kinase called Akt or protein kinase B (Akt/PKB). See Kandel & Hay (1999) Exp. Cell. Res. 253:210-229, which is herein incorporated by reference. Many of the known effects of PI 3-kinase are mediated through Akt/PKB.

As provided in Example 8, serum increases the activated phosphorylated state of Akt/PKB, and LY294002 prevents the serum activation. Additionally, genistein attenuated serum-induced Akt phosphorylation/activation. The combination of LY294002 and DPI inhibits NF-κB activation in a manner similar to the combination of a polyphenolic compound and DPI. The combination a ROS inhibitor and the PI 3-kinase inhibitor, LY294002, inhibit NF-κB activation. As indicated above, inhibition of NF-κB activation can, in turn, sensitize the cancer cell to apoptosis.

Therefore, the present invention provides a preventing, inhibiting, or attenuating the activation of Akt/PKB in a cell comprising administering to the cell at least one polyphenolic compound or a ROS inhibitor and a PI 3-kinase inhibitor, or at least one polyphenolic compound and an inhibitor of NADPH oxidase or inhibitor of ROS formation.

Thus, the present invention provides methods of treating, preventing, or inhibiting cancer in a subject comprising administering to the subject at least one polyphenolic compound. The methods of the present invention may further comprise one or more of the following:

1. Administering a ROS inhibitor;

2. Administering a PI 3-kinase inhibitor;

3. Administering a NADPH oxidase inhibitor;

4. Preventing or inhibiting NF-κB activation;

5. Inducing apoptosis;

6. Inducing caspase-3 activation;

7. Inducing mitochondrial cytochrome c release;

8. Inducing dissipation of mitochondrial polarity; and

9. Activating mitochondrial PTP; and

10. Activating PARP.

G. Rottlerin: A Plant-Derived Polyphenolic Compound

As provided herein, rottlerin is a plant derived polyphenolic compound that is more potent than other polyphenolic compounds and may be used alone in order to prevent, treat, or inhibit proliferative diseases such as cancers including pancreatic cancer; and inflammatory diseases in which NF-κB is involved in the pathogensesis such as pancreatitis, and other diseases or disorders associated with NF-κB activation. Rottlerin causes apoptotic cell death of pancreatic cancer cells cultures in vitro by “opening” the cancer cell's mitochondrial permeability transition by (1) releasing cytochrome c from the mitochondria, (2) activating cellular caspases, (3) inhibiting the formation of ROS, and (4) inhibiting the activation of NF-κB. Therefore, the present invention provides methods of using rottlerin or a derivative thereof to prevent, treat, or inhibit cancers and cancer recurrence. The present invention also provides methods of using rottlerin to sensitize cancers to chemotherapy, radiotherapy, thermal therapy, and the like known in the art because the major reason for the lack of efficacy of these therapies is that cancers can become resistant to these therapies because the cancer cell upregulates survival factors, i.e. NF-κB, to prevent it from undergoing apoptosis. See Soltoff (2001) J. Biol. Chem. 276:37986-37992, which is herein incorporated by reference.

Rottlerin, a polyphenolic phytochemical derived from the plant Mallotus philippinensis, has the following structural formula:

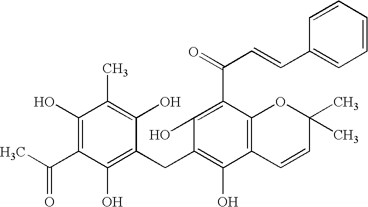

As provided herein, rottlerin derivatives have the following general structural formula:

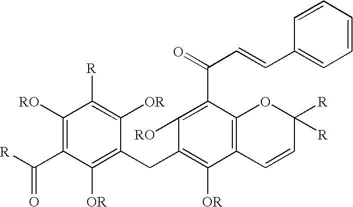

wherein each R are independently selected from the group consisting of hydrogen, hydroxyl, a halo, alkyl, or alkoxyl. In some preferred embodiments, the alkyl may be methyl or ethyl. In some preferred embodiments, the alkoxyl groups may be methoxyl or ethoxyl. In some embodiments, the halo is fluoro. In some embodiments, the ring structures of either the rottlerin compound or the rottlerin derivatives according to the present invention may be optionally substituted.

As used herein, a “halo” means a halogen radical such as fluoro, chloro, bromo or iodo.

As used herein, an “alkyl” is intended to mean a straight or branched chain monovalent radical of saturated and/or unsaturated carbon atoms and hydrogen atoms, such as methyl (Me), ethyl (Et), propyl (Pr), isopropyl (i-Pr), butyl (n-Bu), isobutyl (i-Bu), t-butyl (t-Bu), (sec-Bu), ethenyl, pentenyl, butenyl, propenyl, ethynyl, butynyl, propynyl, pentynyl, hexynyl, and the like, which may be unsubstituted (i.e., contain only carbon and hydrogen) or substituted by one or more suitable sustituents as defined below (e.g., one or more halogen, such as F, Cl, Br, or I, with F and Cl being preferred).

As used herein, a “hydroxyl” is intended to mean the radical —OH.

As used herein, an “alkoxyl” is intended to mean the radical —OR^{a}, where R^{a} is an alkyl group. Exemplary alkoxyl groups include methoxyl, ethoxyl, propoxyl, and the like.

In general, the various moieties or functional groups for variables in the formulae may be “optionally substituted” by one or more suitable “substituents”. The term “substituent” or “suitable substituent” is intended to mean any suitable substituent that may be recognized or selected, such as through routine testing, by those skilled in the art. Illustrative examples of useful substituents are those found in the exemplary compounds that follow, as well as halogen (chloro, iodo, bromo, or fluoro); C₁₋₆-alkyl; C₁₋₆-alkenyl; C₁₋₆-alkynyl; hydroxyl; C₁₋₆ alkoxyl; amino; nitro; thiol; thioether; imine; cyano; amido; phosphonato; phosphine; carboxyl; carbonyl; aminocarbonyl; thiocarbonyl; sulfonyl; sulfonamine; sulfonamide; ketone; aldehyde; ester; oxygen (═O); haloalkyl (e.g., trifluoromethyl); carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl); carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); nitro; thiol; thioether, O-lower alkyl; O-aryl, aryl; aryl-lower alkyl; CO₂CH₃; CONH₂; OCH₂CONH₂; NH₂; SO₂NH₂; OCHF₂; CF₃; OCF₃; and the like. Such moieties may also be optionally substituted by a fused-ring structure or bridge, for example OCH₂—O. All of these substituents may optionally be further substituted with a substituent selected from groups such as hydroxyl groups, halogens, oxo groups, alkyl groups, acyl groups, sulfonyl groups, mercapto groups, alkylthio groups, alkyloxyl groups, cycloalkyl groups, heterocycloalkyl groups, aryl groups, heteroaryl groups, carboxyl groups, amino groups, alkylamino groups, dialkylamino groups, carbamoyl groups, aryloxyl groups, heteroaryloxyl groups, arylthio groups, heteroarylthio groups, and the like.

The term “optionally substituted” is intended to expressly indicate that the specified group is unsubstituted or substituted by one or more suitable substituents, unless the optional substituents are expressly specified, in which case the term indicates that the group is unsubstituted or substituted with the specified substituents. As defined above, various groups may be unsubstituted or substituted (i.e., they are optionally substituted) unless indicated otherwise herein (e.g., by indicating that the specified group is unsubstituted).

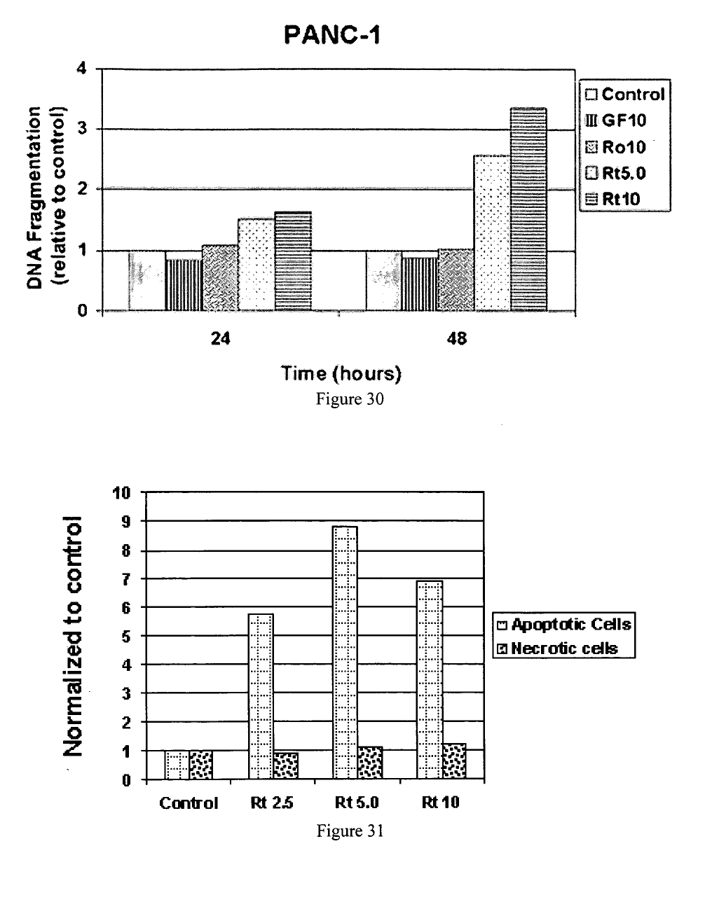

Rottlerin has been used as an inhibitor of protein kinase C δ (PKCδ) and has recently been reported to have mitochondrial effects when used in experiments to define the role of PKC δ in certain cellular functions. See Arlt et al. (2003) Oncogene 22:32-43-3251, which is herein incorporated by reference. 11791 The effects of rottlerin and two inhibitors of PKC, GF109203X (GF) and Ro-32-0432 (Ro), on apoptosis in MIA PaCa-2 and PANC-1 pancreatic cancer cells were determined by measuring their effects on oligonucleosomal DNA fragmentation, a specific measure of apoptosis according to Example 9. FIG. 29 shows that rottlerin, but not PKC inhibitors, cause apoptosis in MIA PaCa-2 pancreatic cancer cells. FIG. 30 shows that rottlerin, but not PKC inhibitors, cause apoptosis in PANC-1 pancreatic cancer cells.

The effects of rottlerin on apoptosis were confirmed by using Annexin V staining according to Example 9. FIG. 31 shows that rottlerin causes apoptosis as determined by this double staining technique. Thus, FIGS. 29-31 indicate that rottlerin causes apoptosis in pancreatic cancer cells in a manner independent of PKC inhibition.

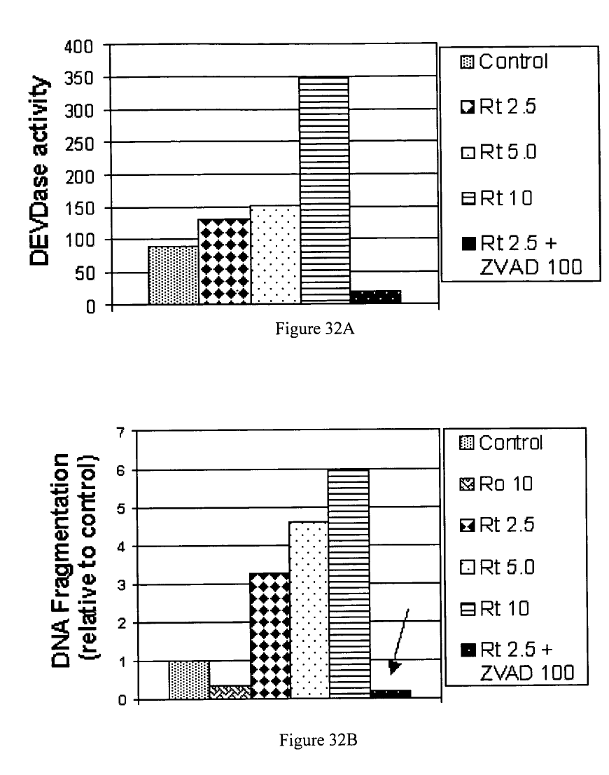

To demonstrate the role of caspases in the mechanism action of rottlerin to cause apoptosis in the pancreatic cancer cells, its effects on caspase-3 activity, i.e. DEVDase activity, as well as the effect of the broad-spectrum caspase inhibitor, Z-VAD, on apoptosis by measuring the effect of caspase inhibition on oligonucleosomal DNA fragmentation caused by rottlerin was examined as provided in Example 9. As shown in FIG. 32, caspase-3 activity is markedly activated by rottlerin and that Z-VAD by inhibiting the activation of the caspase reverses the apoptosis-inducing effect of rottlerin as measured by oligonucleosomal DNA fragmentation.

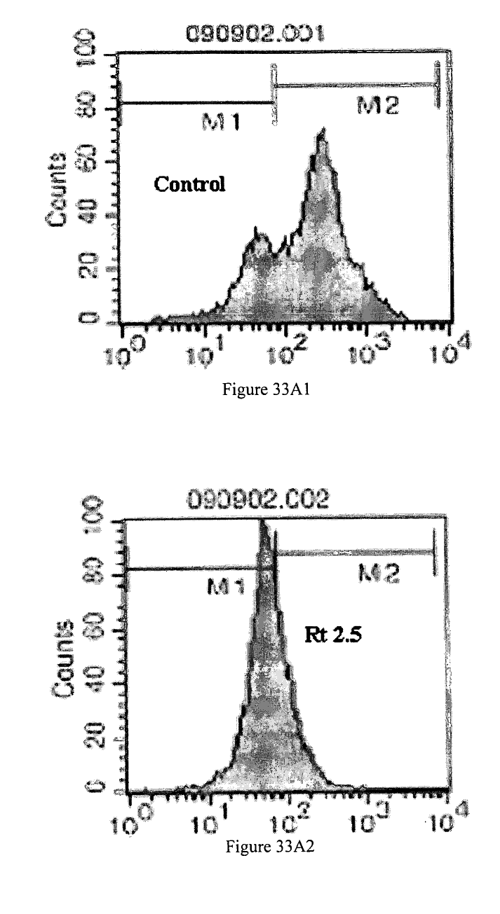

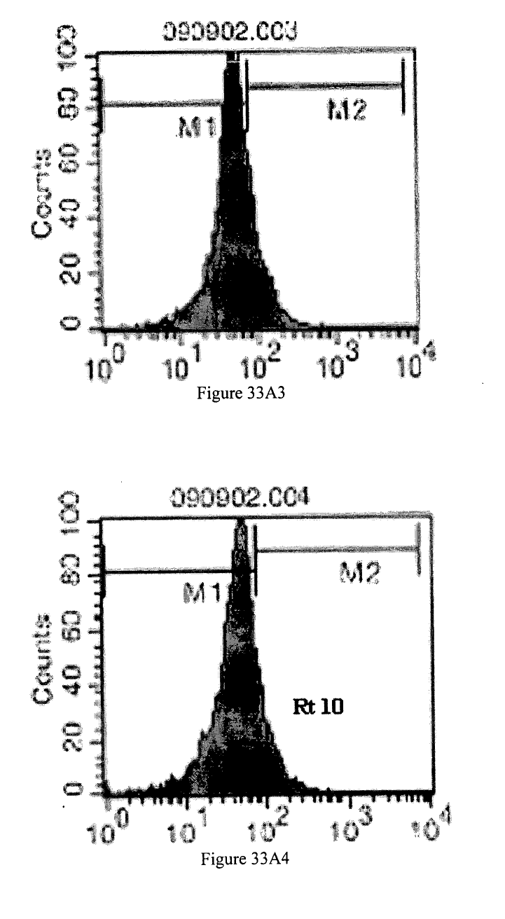

In order to determine the effects of rottlerin on apoptosis were due to its ability to cause mitochondrial dysfunction in the pancreatic cancer cells, its effect on mitochondrial membrane potential, a measure of mitochondrial dysfunction, was conducted according to Example 9. FIG. 33 shows a marked effect of rottlerin on inhibiting the mitochondrial potential. Mitochondrial dysfunction results in release of its cytochrome c release, which in turn activates caspases and apoptosis.

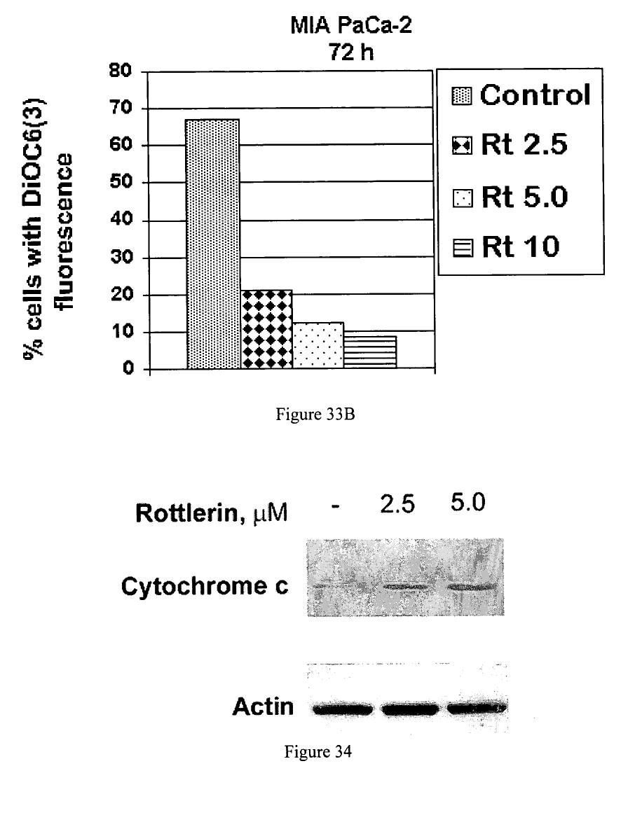

To determine if rottlerin causes mitochondrial cytochrome c release, the effect=of rottlerin on mitochondrial cytochrome c realease in MIA PaCa-2 pancreatic cancer cells was analyzed according to Example 9. FIG. 34 shows that rottlerin does indeed cause the release of cytochrome c from the mitochondria into the cytoplasm of the cancer cell.

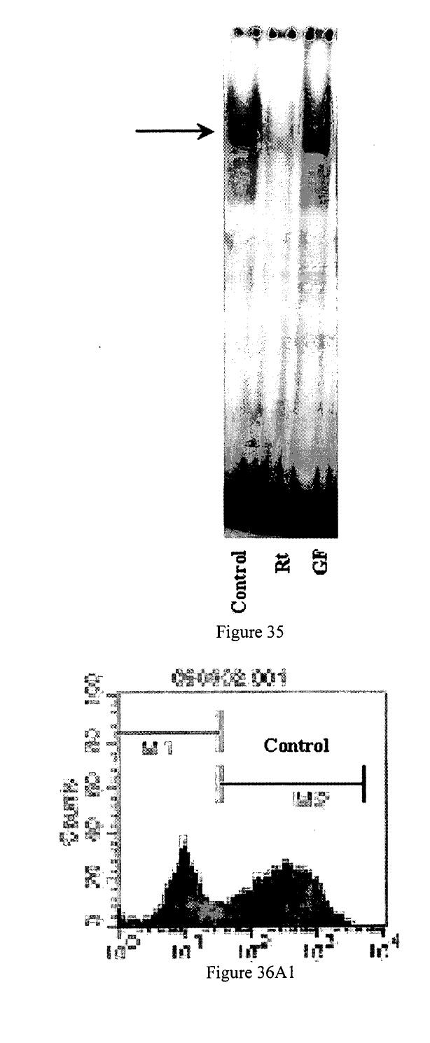

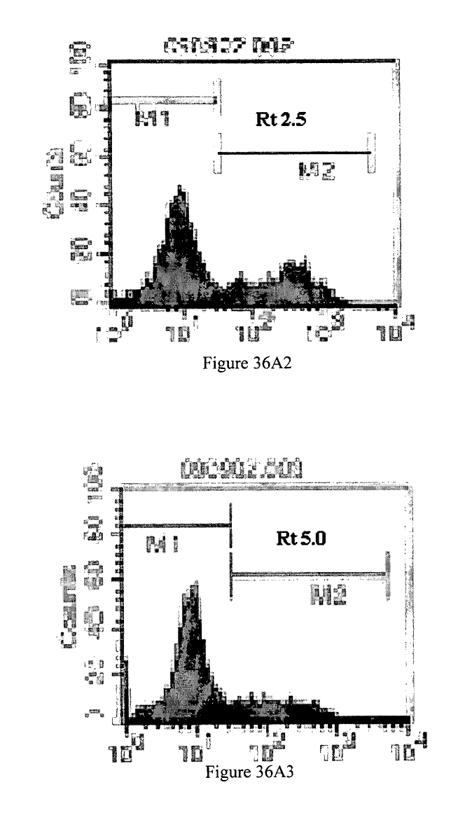

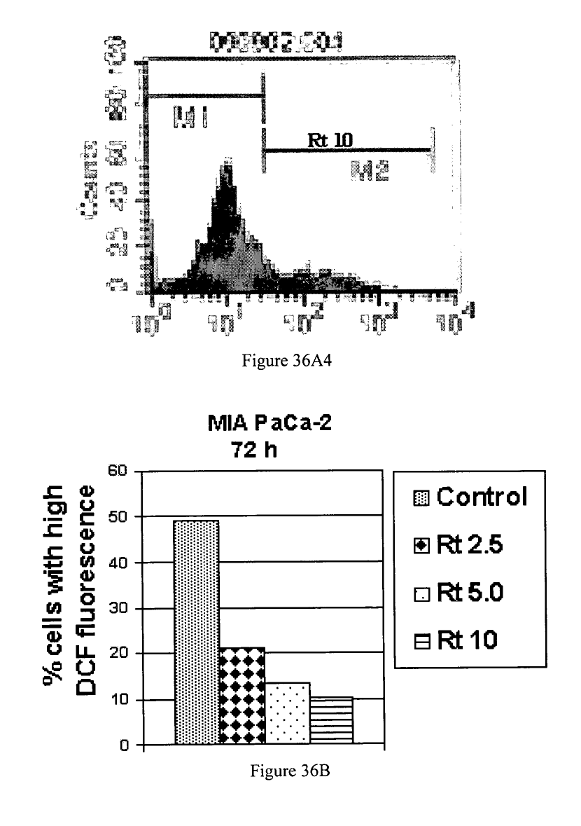

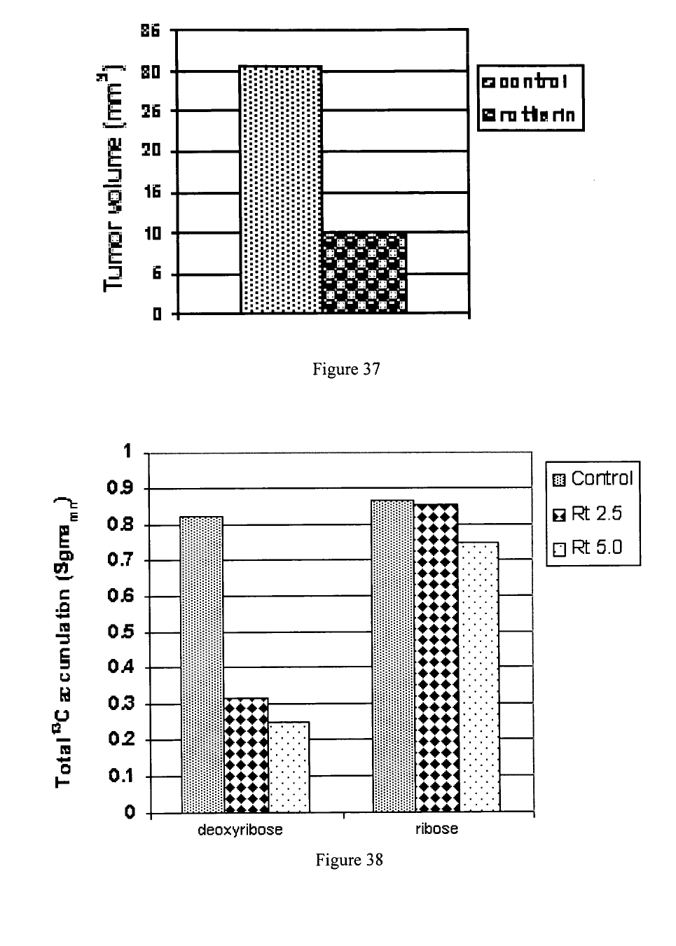

The experiments according to Example 9 were designed to determine the effects of rottlerin on NF-κB activation and production of ROS, two factors that promote survival in pancreatic cancer cells by preventing apoptosis. As illustrated in FIG. 35 and FIG. 36, rottlerin but not the PKC inhibitor prevented the activation of NF-ICB in the cancer cells and rottlerin decreased the formation of ROS in the cancer cells. Having demonstrated that rottlerin causes apoptosis in human pancreatic cancer cells in vitro, in vivo experiments were conducted using a subcutaneous human pancreatic cancer model according to Example 9. See FIG. 37. These studies were designed to evaluate whether the potent pro-apoptotic properties of rottlerin in vitro translate to a reduction in tumor growth in vivo. These studies in nude mice demonstrated that daily intraperitoneal injection of rottlerin at 0.5 mg/kg body weight for 10 days had no gross toxic effects. Animals treated with either control vehicle or rottlerin similarly gained weight during the treatment period.

To evaluate the effects of rottlerin treatment on pancreatic cancer growth about 2×10⁶ MIA PaCa-2 cells were injected subcutanously into the flank of nude mice. After the tumor cells formed a tumor of about 2×2×2 mm (after about 3 days) the animals were treated with daily intraperitoneal injection of either rottlerin (0.5 mg/kg body weight) or control vehicle for 14 days. After the treatment period all tumors were harvested and tumor volume was assessed using the formula for a hemi-ellipsoid (2/3*π*a*b*c with a, b, and c being the half diameters for height, width, and length of the tumor). After a 14 days treatment period rottlerin caused a significant reduction in tumor growth with an average tumor volume of about 10.21 mm³, while control tumors reached an average size of about 30.88 mm³. This translates to about a 67% reduction in tumor volume after 14 days. See FIG. 37. These experiments demonstrate that rottlerin in addition to the proapoptotic effects in vitro also exhibits potent anti-tumor effects in subjects.

The effect of rottlerin on deoxyribose and ribose ¹³C tracer accumulation from glucose in MIA PaCa-2 cells was determined according to Example 9. MIA PaCa-2 cells were treated with vehicle, 2.5 μM and 5.0 μM rottlerin for 72 hours in the presence of [1,2-¹³C₂]glucose in culture. As shown in FIG. 38, rottlerin treatment induced a significant decrease in DNA deoxyribose tracer accumulation (left) but did not affect RNA ribose synthesis (right), indicating a distinctive cell cycle arrest with little or no toxicity on RNA synthesis. FIG. 38 represents the mean of 3 cultures in each group. This metabolic phenotype is also seen in early apoptosis when DNA synthesis is halted but RNA synthesis is still active.

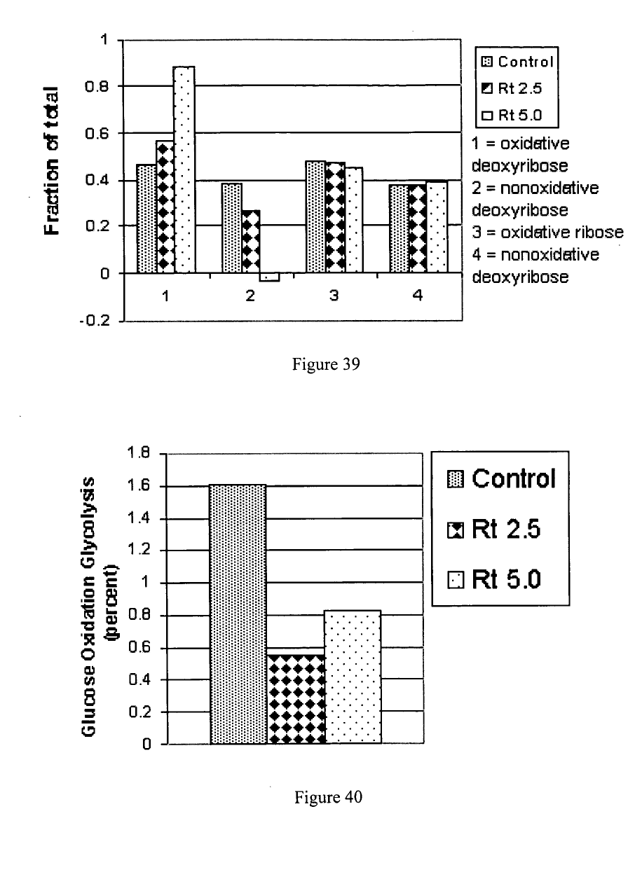

Ribose and deoxyribose molecules labeled with a single ¹³C atom on the first carbon position (m1) recovered from RNA were used to gauge the ribose fraction produced by direct oxidation of glucose through the G6PD pathway according to Example 9. Ribose molecules labeled with ¹³C on the first two carbon positions (m2) were used to measure the fraction produced by the non-oxidative steps of the pentose cycle via transketolase. FIG. 39 shows oxidative and non-oxidative nucleic acid precursor synthesis for DNA and RNA production in response to 2.5 and 5.0 mM rottlerin. The data indicates that rottlerin primarily affects DNA precursor synthesis through the non-oxidative transketolase pathway and there is a dose dependent increase in the oxidative synthesis of deoxyribose. RNA ribose synthesis was not affected by rottlerin treatment indicating that this phytochemical affects metabolic pathways and non-oxidative precursor synthesis during the S cycle phase when DNA is synthesized in rapidly proliferating MIA PaCa-2 cells. The result suggests that inhibiting non-oxidative deoxyribose synthesis is a very effective and selective mechanism of controlling cell proliferation in pancreatic cancer.

The effect of rottlerin on direct glucose oxidation and recycling in the pentose cycle in MIA PaCa-2 cells was determined according to Example 9. The data in FIG. 40 demonstrates that cultured MIA PaCa-2 cells oxidize about 1.6 percent of glucose via the pentose cycle then recycle this substrate back to glycolysis via transketolase and transaldolase. Rottlerin decreased direct glucose oxidation in the pentose cycle and recycling via the non-oxidative steps of the pentose cycle, although this decrease was not dose dependent.

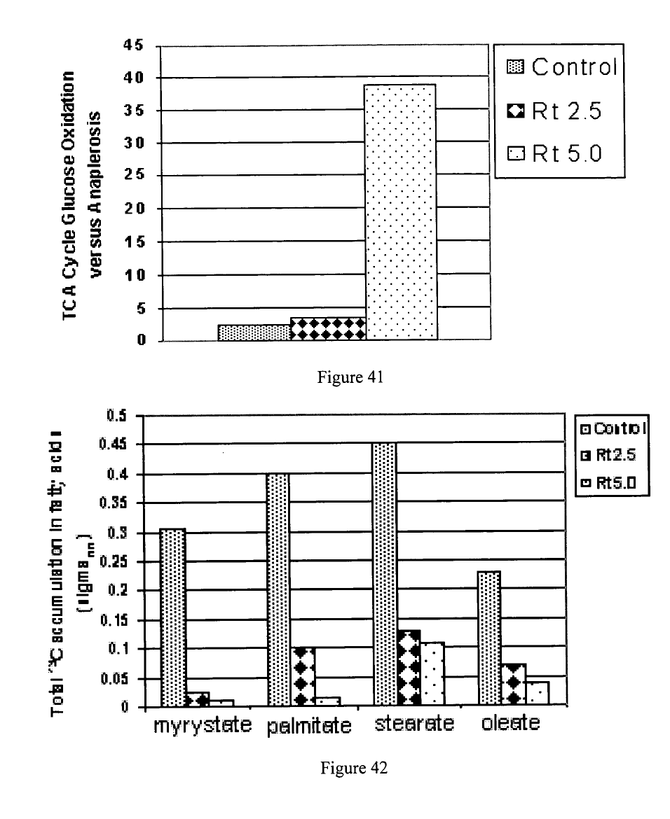

The effect of rottlerin on glucose oxidation relative to glucose anaplerosis in the TCA cycle of MIA PaCa-2 cells was studied according to Example 9. FIG. 41 demonstrates a dose dependent significant increase in glucose oxidation relative to glucose anaplerosis in the TCA cycle based on glutamate stable isotope rearrangements.

The effect of rottlerin on de novo myrystate, palmitate, stearate and oleate fatty acid synthesis of MIA PaCa-2 cells was studied according to Example 9. FIG. 42 indicates that de novo fatty acid synthesis, chain elongation and desaturation from glucose is dramatically inhibited by rottlerin based on the sharp decrease in 13C accumulation into these fatty acid species. This finding is consistent with the limited macromolecule synthesis ability of cycle arrested and apoptotic MIA PaCa-2 cells in response to rottlerin treatment.

For the rottlerin experiments disclosed herein, mass spectral data were obtained on the HP5973 mass selective detector connected to an HP6890 gas chromatograph (Hewlett-Packard, Palo Alto, Calif.). The settings were as follows: GC inlet 250° C., transfer line 280° C., MS source 230° C., MS Quad 150° C. An HP-5 capillary column (30 m length, 250 μm diameter, 0.25 μm film thickness) was used for glucose, ribose and lactate analyses. Because transketolase has the highest metabolic control coefficient in the non-oxidative branch of the pentose cycle, the terms “non-oxidative pentose cycle” and “transketolase” interchangeably herein. See Boros et al. (1997) Cancer Res. 57:4242-4248, which is herein incorporated by reference.

As provided herein, rottlerin dose-(about 2.5 μM to about 10 μM) and time-(about 24 hours to about 72 hours) dependently stimulated apoptosis in MIA PaCa-2 and PANC-1 cells. Rottlerin did not increase necrosis, i.e. cells positive for PI, and did not decrease cellular ATP. The pro-apoptotic effect of rottlerin was much greater than of other polyphenols studied. In particular, 10 μM rottlerin stimulated apoptosis about 9+0.5 fold (n=4) in MIA PaCa-2 cells, whereas 100 μM genistein, resveratrol or quercetin increased the apoptotic rate about 2 to about 3 fold. Other PKC inhibitors (GF109203X and Ro-32-0432) did not stimulate apoptosis although they inhibited PKC δ to the same extent as rottlerin. Thus, the pro-apoptotic effect of rottlerin is not mediated by PKC 6 inhibition alone. ROS serves as survival factor in pancreatic cancer cells and that stimulation of NF-κB is one mechanism of ROS pro-survival action. Rottlerin inhibited both ROS generation and NF-κB binding activity, resulting in pro-apoptotic cytochrome c release, mitochondrial depolarization, and activation of caspase-3. Rottlerin inhibited both ROS and NF-κB to a much greater extent than genistein or resveratrol.

As provided herein, rottlerin causes apoptosis in pancreatic cancer cells by causing mitochondrial depolarization and release of its cytochrome c. The cytochrome c, in turn, causes activation of cellular caspases, which mediate apoptosis. Rottlerin also acts by inhibiting the formation of ROS and by inhibiting the activation of NF-κB. ROS and activated NF-κB act as survival factors for cancer cells. Thus, their inhibition promotes cell death through apoptosis. In addition, rottlerin significantly reduces the growth of pancreatic tumors. Rottlerin treated tumor cells also exhibit a significant decrease in macromolecule DNA/RNA precursor synthesis and that of fatty acids which are molecules are necessary for proliferation and growth of tumor cells. Thus, rottlerin has multiple effects on the cancer cells that promote their death while having no significant toxic effects on normal tissues in vivo.

Therefore, the present invention provides methods of inducing apoptosis in cancer cells, such as pancreatic cancer cell, which comprises contacting the cells with rottlerin. The present invention also provides methods of treating, preventing, or inhibiting tumor growth in a subject which comprises administering to the subject a therapeutically effective amount of rottlerin.

H. Rottlerin and Combination Therapies for Diseases Associated with NF-κB Activation

Rottlerin may be used in combination with other therapies for treating, preventing, or inhibiting diseases and disorders associated with NF-κB activation. For example, rottlerin may be used in combination with other chemotherapeutic agents such as gemcitabine, the most effective chemotherapeutic for pancreatic cancer. See Li et al. (2004) Lancet 363(9414):1049, which is herein incorporated by reference. Other chemotherapeutics may be used in combination with rottlerin. One skilled in the art may readily ascertain the effectiveness and suitable dosages of the combinations according to the methods disclosed herein as well as methods known in the art.

Since rotterlin inhibits NF-κB activation which is involved in abnormal cell proliferation such as cancer and inflammatory diseases such as pancreatitis, rottlerin may be used alone or in combination with antiproliferative agents and anti-inflammatory agents known in the art to treat both cancer and inflammatory disorders.

Antiproliferative agents include asparaginase, alemtuzumab, bleomycin, busulfan, beracizumab, carboplatin, cisplatin, cetuximab, cyclophosphamide, daunorubicin, docetaxel, epirubicin, floxuridine, fluoruracil, foscarnet, gentuzamab izogamicin, hydroxyurea, idarubicin, ifosfamide, iriotecan, lomustine, leamisole, melphalan, mercaptopurine, methotrexate, methyl CCNU, oxoliplatin, paclitaxel, rituximab, streptozocin, tamoxifen, temozolomide, tenipozide, thioguanine, thiotepa, tumor necrosis factor, tositumomab, trastuzmab, vinblastine, vincristine, 9-aminocamptotheca, 90Y ibritunomab tiuxetan, growth factor inhibitors, and the like.

Antiinflammatory agents include acetylsalicylic acid, aspirin, Ecotrin, choline magnesium salicylate, Trilisate, Cox-2 inhibitors, diclofenac, Voltaren, Cataflam, Voltaren-XR, diflunisal, Dolobid, etodolac, Lodine, fenoprofen, Nalfon, flurbiprofen, Ansaid, ibuprofen, Advil, Motrin, Medipren, Nuprin, indomethacin, Indocin, Indocin-SR, ketoprofen, Orudis, Oruvail, meclofenamate, Meclomen, nabumetone, Relafen, naproxen, Naprosyn, Naprelan, Anaprox, Aleve, oxaprozin, Daypro, phenylbutazone, Butazolidine, salsalate, Disalcid, Salflex, tolmetin, Tolectin, valdecoxib, Bextra, corticosteroids, antiinflammatory cytokine agents, antichemokine agents, Infliximab, and the like.

In addition, rottlerin and an inhibitor of PKCδ translocation, PKCε translocation, or both, may be combined to treat, prevent, or inhibit diseases and disorders mediated by NF-κB activation such as proliferative diseases including cancer and inflammatory diseases including pancreatitis. The present invention provides methods of inhibiting NF-κB activation in a cell or a subject comprising administering to the cell or the subject rottlerin alone or in combination with at least one an inhibitor of PKCδ translocation, PKCε translocation, or both. In some embodiments, the inhibitors include rottlerin, GF109203X (Sigma, St. Louis, Mo.), and the peptide inhibitors provided herein. One skilled in the art may readily ascertain the effectiveness and suitable dosages of the combinations according to the methods disclosed herein as well as methods known in the art.

In accordance with the present invention, at least one polyphenolic compound may be administered in a therapeutically effective amount to a mammal such as a human. A therapeutically effective amount may be readily determined by standard methods known in the art.

An effective amount of a polyphenolic compound is an amount that treats, prevents, or inhibits cancer or tumor growth as compared to a control using methods known in the art. An effective amount of a polyphenolic compound may also mean an amount that induces apoptosis in a cancer cell as compared to a control using methods known in the art. The dosages to be administered can be determined by one of ordinary skill in the art depending on the clinical severity of the disease, the age and weight of the subject, or the exposure of the subject to carcinogens and neoplastic conditions. Preferred effective amounts of the compounds of the invention ranges from about 1 to about 2400 mg/kg body weight, preferably about 10 to about 1000 mg/kg body weight, and more preferably about 10 to about 500 mg/kg body weight. Preferred topical concentrations include about 0.1% to about 10% in a formulated salve.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a compound or composition of the present invention can include a single treatment or, preferably, can include a series of treatments.

In a preferred example, a subject is treated with a compound of the invention in the range of between about 1 to about 2400 mg/kg body weight, at least one time per week for between about 1 to about 24 weeks, and preferably between about 1 to about 10 weeks. It will also be appreciated that the effective dosage of the compound used for treatment may increase or decrease over the course of a particular treatment. Changes in dosage may result and become apparent by standard diagnostic assays known in the art. In some conditions chronic administration may be required.

The pharmaceutical compositions of the invention may be prepared in a unit-dosage form appropriate for the desired mode of administration. The compositions of the present invention may be administered for therapy by any suitable route including oral, rectal, nasal, topical (including buccal and sublingual), vaginal and parenteral (including subcutaneous, intramuscular, intravenous and intradermal). It will be appreciated that the preferred route will vary with the condition and age of the recipient, the nature of the condition to be treated, and the chosen active compound.

It will be appreciated that the actual dosages of the agents used in the compositions of this invention will vary according to the particular complex being used, the particular composition formulated, the mode of administration, and the particular site, host, and disease being treated. Optimal dosages for a given set of conditions may be ascertained by those skilled in the art using conventional dosage-determination tests in view of the experimental data for a given compound. Administration of prodrugs may be dosed at weight levels that are chemically equivalent to the weight levels of the fully active forms.

The polyphenolic compounds of the invention can be incorporated into pharmaceutical compositions suitable for administration. Pharmaceutical compositions of this invention comprise a therapeutically effective amount of a polyphenolic compound, and an inert, pharmaceutically acceptable carrier or diluent. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like. The use of such media and agents for pharmaceutically active substances is well known in the art.

Except insofar as any conventional media or agent is incompatible with the active compound, use thereof in the compositions is contemplated. Supplementary active compounds can also be incorporated into the compositions. Supplementary active compounds include ROS inhibitors such as N-acetylcysteine, vitamins C, A, and E, beta-carotene, allopurinol, carvediol, coenzyme Q, Tiron, DPI, and any other antioxidant or inhibitor of ROS.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of a polyphenolic compound is dissolved in an aqueous solution of an organic or inorganic acid, such as 0.3M solution of succinic acid or citric acid. If a soluble salt form is not available, the compound may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, the polyphenolic compound of the present invention is dissolved in DMSO and diluted with water. The composition may also be in the form of a solution of a salt form of the active ingredient in an appropriate aqueous vehicle such as water or isotonic saline or dextrose solution.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally comprise gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can comprise the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can comprise any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Preferred formulations for oral formulations include microcrystalline tablets, gelatin capsules, or the like.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated comprising a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may comprise formulatory agents such as suspending, stabilizing and/or dispersing agents.

Pharmaceutical compositions suitable for injectable use include sterile aqueous solutions (where water soluble) or dispersions and sterile powders for the extemporaneous preparation of sterile injectable solutions or dispersion. Aqueous injection suspensions may comprise substances which increase the viscosity of the suspension, such as sodium carboxymethyl cellulose, sorbitol, or dextran. Optionally, the suspension may also comprise suitable stabilizers or agents which increase the solubility of the compounds to allow for the preparation of highly concentrated solutions. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium comprising, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating a therapeutically effective amount of a compound of the invention in an appropriate solvent with one or a combination of ingredients enumerated above, as required, followed by filtered sterilization. Generally, dispersions are prepared by incorporating at least one polyphenolic compound into a sterile vehicle which comprises a basic dispersion medium and the required other ingredients from those enumerated above. In the case of sterile powders for the preparation of sterile injectable solutions, the preferred methods of preparation are vacuum drying and freeze-drying which yields a powder of the active compound plus any additional desired ingredient from a previously sterile-filtered solution thereof.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, foams, powders, sprays, aerosols or creams as generally known in the art.

For example, for topical formulations, pharmaceutically acceptable excipients may comprise solvents, emollients, humectants, preservatives, emulsifiers, and pH agents. Suitable solvents include ethanol, acetone, glycols, polyurethanes, and others known in the art. Suitable emollients include petrolatum, mineral oil, propylene glycol dicaprylate, lower fatty acid esters, lower alkyl ethers of propylene glycol, cetyl alcohol, cetostearyl alcohol, stearyl alcohol, stearic acid, wax, and others known in the art. Suitable humectants include glycerin, sorbitol, and others known in the art. Suitable emulsifiers include glyceryl monostearate, glyceryl monoleate, stearic acid, polyoxyethylene cetyl ether, polyoxyethylene cetostearyl ether, polyoxyethylene stearyl ether, polyethylene glycol stearate, propylene glycol stearate, and others known in the art. Suitable pH agents include hydrochloric acid, phosphoric acid, diethanolamine, triethanolamine, sodium hydroxide, monobasic sodium phosphate, dibasic sodium phosphate, and others known in the art. Suitable preservatives include benzyl alcohol, sodium benzoate, parabens, and others known in the art.

For administration to the eye, the compound of the invention is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmaceutically acceptable ophthalmic vehicle may be an ointment, vegetable oil, or an encapsulating material. A compound of the invention may also be injected directly into the vitreous and aqueous humor.

Alternatively, the active ingredient may be in powder form for constitution with a suitable vehicle, e.g., sterile pyrogen-free water, before use. The compounds may also be formulated in rectal compositions such as suppositories or retention enemas, e.g., comprising conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) comprises VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied, for example: other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers comprising the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit comprising a predetermined quantity of active compound calculated to produce the desired therapeutic effect in association with the required pharmaceutical carrier.

The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The polyphenolic compounds of the present invention may be prepared using reaction routes, synthesis schemes and techniques available in the art using starting materials that are readily available.

The following examples are intended to illustrate but not to limit the invention.

### EXAMPLE 1

### Pancreatic Cancer Growth Assay

To determine the effect of a polyphenolic compound on pancreatic cancer cell growth, the following assay using a nude mouse model was conducted. Specifically, the effect of quercetin was tested in a nude mouse model of pancreatic cancer using the highly malignant pancreatic cancer cell line, Mia PACA-2.

Tumor induction in nude mice was performed as described by Hotz et al. (2001) Pancreas 22:113-121, which is herein incorporated by reference. For subcutaneous tumor formation, 1×10⁷ Mia PACA-2 tumor cells were subcutaneously injected in the medio-dorsal region of a nude mouse. After 4 weeks a small tumor fragment, about 1 mm in diameter, was removed from the subcutaneous tumor and transplanted into the pancreatic tail of mice of two study groups. Ten days after tumor transplantation, treatment with or without quercetin was initiated. The treated animals received daily intraperitoneal injections of 1.3 mg quercetin dissolved in DMSO; the control group received DMSO only by intraperitoneal injection. The animals were sacrificed once clinical tumor signs including severe cachexia ascites with abdominal distension or heavy tumor burden, larger than 1.5 cm, became apparent.

Tumor volume was calculated as described by Hotz et al. (2001) as 0.5×length×width×depth. Metastatic tumor spread was determined macroscopically at autopsy in all thoracic, abdominal, retroperitoneal and pelvic organs. All macroscopic suspicious lesions were further confirmed as tumor dissemination by microscopic analysis. Each value in the metastatic score represented a different organ of metastatic tumor spread.

As indicated in Table I, the quercetin treatment had multiple effects on the in vivo growth of the tumor.

TABLE 1
Effect of Treatment with Quercetin on Tumor Progression In Vivo¹

| Parameters of cancer development | Control | Quercetin |
|---|---|---|
| Survival, days | 66.60 ± 3.20 | 75.00 ± 2.50* |
| Tumor volume, cm³ | 5.76 ± 0.92 | 1.56 ± 0.27* |
| Number of metastatic sites | 4.40 ± 0.90 | 0.62 ± 0.26* |
| Percentage of apoptosis | 3.30 ± 0.60 | 7.10 ± 1.00* |

¹Survival in control animals was measured as the number of days after transplantation until the animal died or appeared severely ill. Survival in the quecetin-treated animals was measured as the number of days after transplantation until the animal appeared ill from abdominal distension. The distension was due to dilation of the small and large bowels. The values represent means ± SE, n = 8.-*p < 0.05 compared to untreated animals.

Additionally, the mean number of organs with metastatic lesions was 4.4 in control animals as compared to the 0.6 in quercetin-treated animals. Therefore, quercetin treatment prevents metastatic cancer lesions. Furthermore, quercetin treatment significantly decreased the growth of the primary tumor.

### EXAMPLE 2

### Effects of Serum and Growth Factors

To determine the effect of serum in cancer cells, the following assay was conducted. Mia PACA-2 pancreatic cancer cells were cultured for 72 hours in the absence and presence of serum (15% FBS) or 100 ng/ml insulin growth factor-1 (IGF-1). Dichloroflyorescein diocetate (DCF-DA) was used to label the cells. Intracellular H₂O₂ was measured by flow cytometry of DCF-labeled cells.

Intracellular ROS was measured using oxidation-sensitive cell-permeable fluorescent probe, dichlorofluorescein diacetate (DCF-DA) to measure H₂O₂. See Royall & Ischiropoulos (1993) Arch. Biochem. Biophys. 302:348-355. To measure ROS, cells were collected after incubation, washed with PBS, and incubated for 15 minutes with 8 mM DCF-DA. Samples were analyzed by flow cytometry. The amount of DCF-DA fluorescence correlated with the amount of ROS in the cells

To determine the effects of serum, IGF-1, polyphenols and inhibitors of ROS on ROS production in cancer cells, the following assay was conducted. Mia PACA-2 cells were cultured for 72 hours in the presence of serum or IGF-1 with or without antioxidants, intracellular superoxide scavenger tiron (10 mM) or NADPH oxidase inhibitor, diphenylene iodonium (DPI, 15 μM) and trans-resveratrol (100 μM), genistein (100 μM), or a combination of polyphenolic compounds and antioxidants. Intracellular H₂O₂ was measured by flow cytometry of DCF-labeled cells.

Mia PACA-2 cells and BSp73AS cells were cultured in Dulbecco's Modified Eagle's Medium supplemented with 10% heat inactivated FBS, penicillin G (100 U/ml) and streptomycin (100 mg/ml) in a humidified atmosphere comprising 5% (v/v) CO₂. When the cells were 90% confluent they were detached, washed with alternating centrifugation and resuspension, plated and incubated in the same media with or without FBS or IGF-1 and with or without a given polyphenolic compound and with or without a given ROS inhibitor.

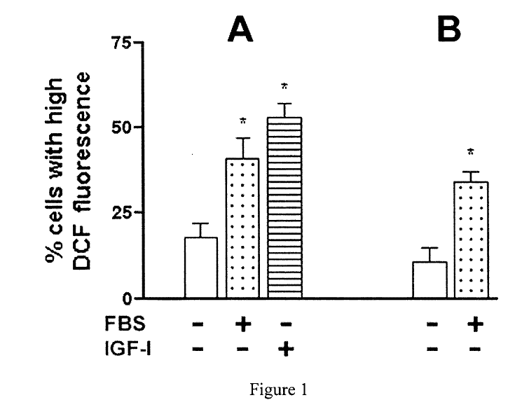

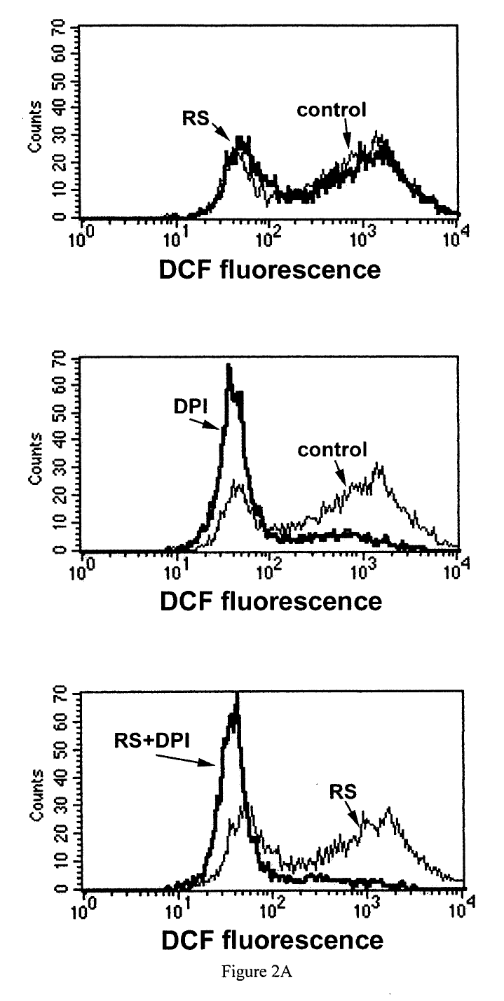

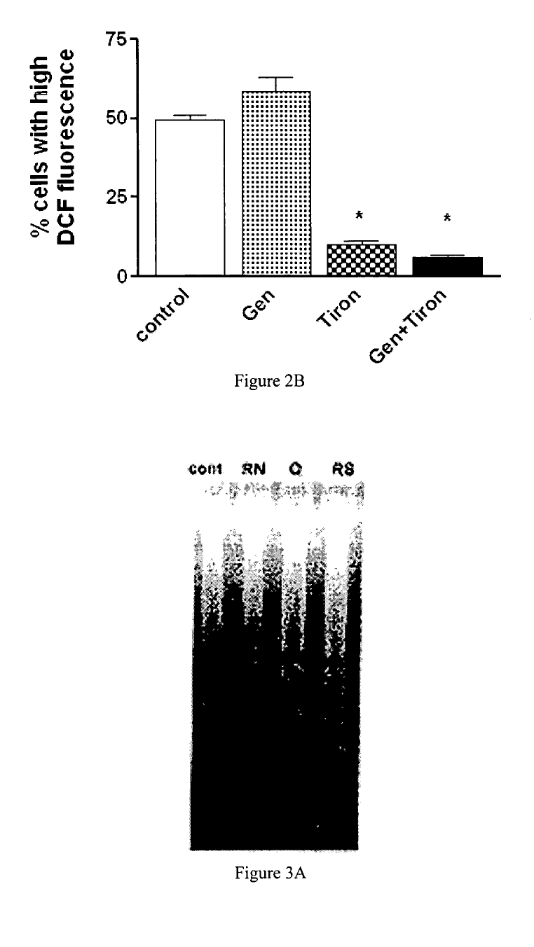

As shown in FIG. 1, the presence of serum and insulin growth factor-1 (IGF-1) increases the percentage of cells with a high DCF fluorescence value. As shown in FIG. 2, antioxidants, but not trans-resveratrol and genistein inhibit production of ROS in Mia PACA-2 pancreatic cancer cells. As illustrated in FIG. 2A, the addition of agents that decrease the production of ROS caused a decrease in the percentage of cells that were highly fluorescent. Additionally, trans-resveratrol and genistein caused small increase in ROS production, which were prevented by DPI.

### EXAMPLE 3

### Apoptosis Assays

I. Polyphenolic Compound Alone

In order to determine the mechanism of the suppressive effects of quercetin on the growth of the pancreatic cancer, apoptosis in the primary tumors using the TUNEL assay was conducted. See Gukovskaya et al. (1997) Clin Invest 100: 1853-1862; Gukovskaya et al. (1996) Gastroenterology 110:875-884; and Sandoval et al. (1996) Gastroenterology 111:1081-1091, which are herein incorporated by reference. Specifically, 3 μm tissue section were deparaffinized and rehydrated through a graded series of ethanol and redistilled water. Tissue sections were refixed in 4% paraformaldehyde for 15 minutes at room temperature and then incubated with proteinase K (20 μg/ml in 10 mM Tirs/HCL, pH 7.4-8.0) for 15 minutes at 37° C. DNA breaks were then labeled with terminal deoxytransferase (TdT) and biotinylated deoxyUTP. Staining without TdT enzyme or the biotinylated substrate were used as negative controls. For positive controls, slides were treated with DNase I. Measurements were made by light microscpy observations and values calculated as the percentage of cells positively stained as a percentage of the total number of cells.

Also as shown in Table I above, there was a significant increase in the percentage of cells undergoing apoptosis in the quercetin-treated animals as compared to the control animals. In contrast to the effect of the quercetin treatment on apoptosis in the tumor tissue, there was no increase in apoptosis detected by the TUNEL assay in normal tissues (data not shown), thereby indicating that the effect of quercetin on apoptosis is tumor tissue specific.

To confirm the apoptosis in vivo results, the effects of quercetin, rutin, and trans-resveratrol in other apoptosis assays in Mia PACA-2 cells and BSp73AS cells in culture were conducted. BSp73AS cells are derived from a rat pancreatic carcinoma and both Mia PACA-2 and BSp73AS cells have mutated p53 and express K-ras. Human pancreatic carcinoma cell line Mia PACA-2 and rat pancreatic carcinoma BSp73AS were cultured in Dulbecco's Modified Eagle's Medium supplemented with 10% heat inactivated FBS, penicillin G (100 U/ml) and streptomycin (100 mg/ml) in a humidified atmosphere comprising 5% (v/v) CO₂. When cells were 90% confluent they were detached, washed with alternating centrifugation and resuspension, plated and incubated in the same media without FBS and with the indicated concentrations of polyphenolic compounds or vehicle for up to 96 hours. Oligonucleotide DNA fragmentation, annexin staining, and PARP proteolysis assays were conducted as follows:

A. Oligonucleotide DNA Fragmentation

BSp73AS pancreatic cancer cells were cultured for 6 hours in the presence or absence of 100 μM of rutin, quercetin, or trans-resveratrol. DNA was isolated as described by Gukovskaya AS, et al. (1997) Clin Invest 100:1853-1862, which is herein incorporated by reference. Briefly, pancreatic cancer cells growing on plates were removed by treatment with trypsin, collected by centrifugation and lysed by resuspension in a buffer comprising 10 mM Tris/HCl (pH 8.0) 10 mM NaCl, 10 mM EDTA, 300 μg/ml proteinase K and 1% SDS. Cell lysates were incubated overnight at 45° C.; and DNA was purified by phenol/chloroform extraction (1:1 v/v), precipitated overnight at 20° C. with 0.3 M sodium acetate and collected by centrifugation at 15,000 g for 15 minutes at 4° C. The pellet comprising RNA and DNA was resuspended in TE buffer (10 mM Tris/HCl (pH 8.0), 1.0 mM EDTA) and treated subsequently with RNase (200 μg/ml) for 2 hours at room temperature, followed by an incubation overnight with proteinase K (200 μg/ml) at 45° C. Finally, the mixture was re-extracted with phenol/chloroform and chloroform, precipitated with ethanol and resuspended in TE buffer. DNA fragments were separated electrophoretically on 1.8% agarose gel comprising 0.5 μg/ml ethidium bromide in 0.5×TBE buffer (TBE: 89 mM Tris base, 89 mM boric acid and 2 mM EDTA). The experiment was repeated twice with similar results.

B. Annexin Staining

Mia PACA-2 cells were cultured for 72 hours in the presence of 0, 12, 24, 50, and 100 μM rutin, quercetin, or trans-resveratrol. About 1×10⁶ cells as determined with a hemocytometer were analyzed for annexin-V binding using an Annexin V-FLUOS Staining Kit (Boehringer Mannheim, Germany). Briefly, cells were washed twice with PBS and incubated for 10 minutes at room temperature with fluorescein isothiocyanate (FITC)-conjugated, annexin-V reagent (20 μg/ml) and propidium iodide (50 μg/ml). Cells were analyzed on a FACScan flow cytometer (Becton Dickinson Immunocytometry System, San Jose, Calif.) equipped with a 15 nW air-cooled 488 nm argon-ion laser. Annexin-V positive and propidium iodide negative cells were considered as apoptotic.

C. PARP Proteolysis

BSp73AS cells were cultured for 6 hours and Mia PACA-2 cells were cultured for 24 hours in the presence or absence of 100 μM of each rutin, quercetin or trans-resveratrol and with or without 50 μM of each K-VAD FMK(K-VAD). The cells were washed twice with PBS and lysed by incubating for 20 minutes at 4° C. in lysis buffer comprising 0.15 M NaCl, 50 mM Tris (pH 7.2), 1% deoxycholic acid (wt/vol), 1% Triton X-100 (wt/vol), 0.1% SHS (wt/vol) and 1 mM PMSF, as well as 5 μg/ml each of protease inhibitors, pepstatin, leupeptin, chymostatin, antipain, and aprotinin. Then the cell lysates were centrifuged for 20 minutes at 15,000 g at 4° C. The supernatants were separated by 4-20% SDS-PAGE for 2 hours at 120 V using precast Tris-glycine gels and a Mini-Cell gel apparatus (Novex, San Diego, Calif.). Separated proteins were electrophoretically transferred to a nitrocellulose membrane for 2 hours at 30 V using a Novex Blot Module (Novex, San Diego, Calif.). Nonspecific binding was blocked by 1 hour incubation of nitrocellulose membranes in 5% (wt/vol) nonfat dry milk in Tris-buffered saline (TBS; pH 7.5). Blots were then incubated overnight at 4° C. with rabbit polyclonal antibody against poly (ADP-ribose) polymerase (PARP) (Santa Cruz Biotechnology, Santa Cruz, Calif.) (1;3,000) in an antibody buffer comprising (1% (wt/vol) non-fat dry milk in TTBS (0.05% vol/vol) Tween-20 in TBS), washed 3 times with TTBS and finally incubated for 1 hour with a peroxidase-labeled secondary antibody in the antibody buffer. Blots were developed for visualization using ECL detection kit. To test for equal protein loading, the blots were stripped and re-probed with an antibody against tubulin. When processing of a protein was measured, a decrease in unprocessed full-length form was measured concomitantly with the increase in the cleaved, active form. The experiment was repeated 3 times with similar results.

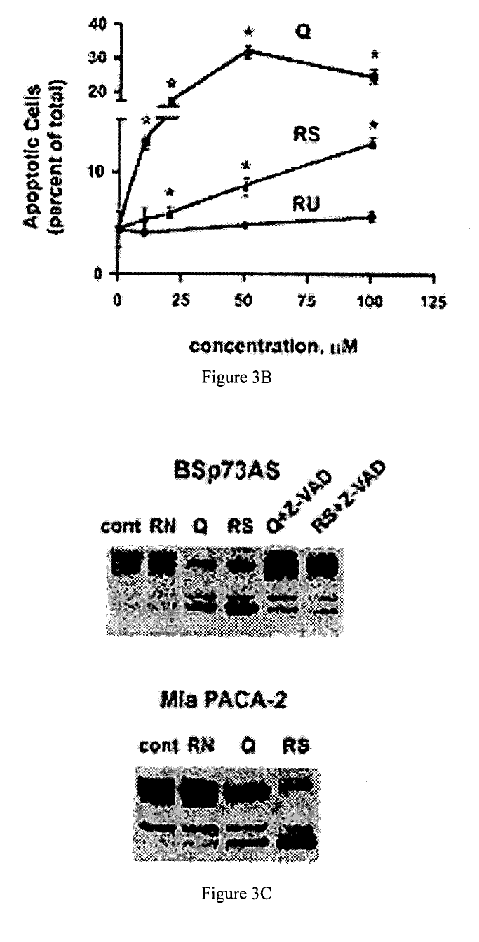

As indicated in FIGS. 3A and 3B, quercetin and trans-resveratrol, but not rutin, caused an increase in oligonucleosomal DNA fragmentation, a unique characteristic of apoptosis, as well as increase annexin staining. Annexin staining is a measure of externalization of phosphatidylserine to the outer plasma membrane leaflet representing another unique characteristic of apoptosis. The dose-response evaluation in FIG. 3B indicates that quercetin is more potent in causing apoptosis than trans-resveratrol. Finally, FIG. 3C illustrates that a protein target of caspases-3 activation, PARP, was cleaved to its activated form in cell lines treated with quercetin and trans-resveratrol, but not rutin. The cleavage did not occur in the presence of the specific caspase inhibitor, Z-VAD. These effects of quercetin and trans-resveratrol on apoptosis occurred independent of the presence of serum in the incubation media.

Therefore, quercetin and trans-resveratrol activate apoptosis in pancreatic cancer cells and indicate that the beneficial effect of quercetin in vivo is due to the ability to cause apoptosis.

II. Combinations

To evaluate the effects of polyphenolic compounds alone and in combination with ROS inhibitors on apoptosis in pancreatic cancer cells, the following assays were conducted.

Mia PACA-2 cells were cultured for 72 hours in the presence of serum with or without 15 μM DPI, 10 mM Tiron, 100 μM genistein, 100 μM or 50 μM trans-resveratrol, or a combination thereof. Oligonucleosomal DNA fragmentation was measured in cell lysates by cell death ELISA.

Mia PACA-2 cells were cultured for 72 hours in the presence of serum with or without 10 mM Tiron, 100 μM trans-resveratrol, or a combination thereof. Phosphatidylserine externalization was measured by flow cytometry in cells stained with Annexin V and propidium iodide (PI). Cells positive for Annexin V (AnV) and negative for PI were considered apoptotic.

Annexin staining was conducted as described above. Cells were collected, washed with PBS, and centrifuged for 10 min at 200×g. DNA fragmentation was determined using Cell Death Detection ELISA Plus kit (Roche Molecular Biochemicals).

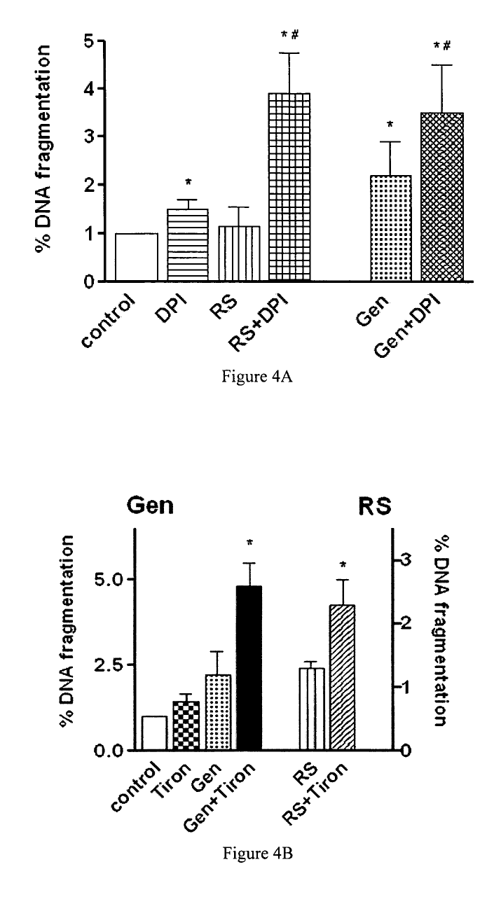

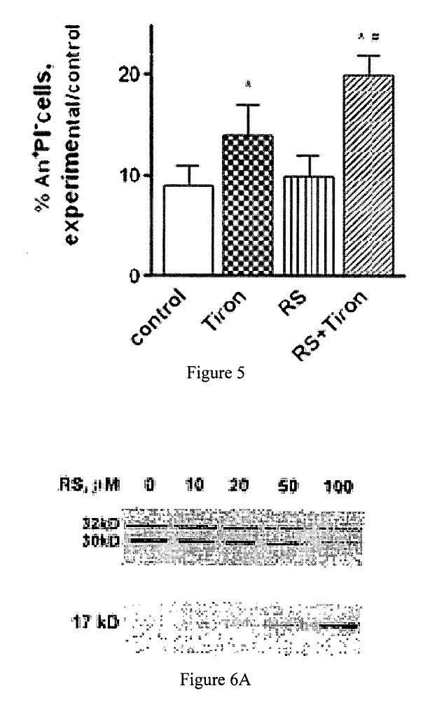

As shown in FIG. 4, the combination of ROS inhibitors and polyphenolic compounds caused oligonucleosomal DNA fragmentation in Mia PACA-2 pancreatic cancer cells. Specifically, at the concentrations used, DPI, trans-resveratrol, and genistein had either no or small effects on oligonucleosomal DNA fragmentation. However, the combination of DPI with either polyphenolic compound resulted in synergistic increases in DNA fragmentation. These results were confirmed by the similar results obtained by the Annexin V staining which are illustrated in FIG. 5.

### EXAMPLE 4

### Caspase-3 Assays

I. Polyphenolic Compound Alone

In order to determine the effect polyphenolic compounds have on caspase-3 activity, the following assay was conducted. BSp73AS cells were culture in the absence of serum or growth factors for 24 hours in 0, 10, 20, and 50 μM of trans-resveratrol and Mia PACA-2 cells were cultured for 72 hours in the presences of 0, 10, 20, and 50 μM of tran-resveratrol. Cell lysates were then prepared and 50 μg protein aliquots were loaded per lane and blotted with rabbit polyclonal antibody against caspase-3 (Santa Cruz Biotechnology, Santa Cruz, Calif.). The experiment was repeated 3 times with similar results.

To determine whether a polyphenolic compound activates caspase-3 activity in a time-dependent manner, the following assay was conducted. BSp73AS cells were cultured for 0, 1, 3, and 6 hours and Mia PACA-2 cells were cultured in the absence of serum or growth factors for 0, 1, 4, 6, and 24 hours in the presence of 100 μM or quercetin, trans-resveratrol, rutin, or control. Caspase-3 activity was measured in cell lysates with a fluorogenic assay using DEVD-AMC as a substrate. The results were normalized to the DEVDase activity in untreated cells.

To determine whether quercetin activates caspase-3 activity in a dose-dependent manner, the following assay was conducted. Cells were cultured for 6 hours in the absence of serum or growth factors in the presence of 0, 10, 20, 50, and 100 μM of quercetin. Caspase-3 activity was measured in cell lysates with a fluorogenic assay using DEVD-AMC as a substrate. The results were normalized to the DEVDase activity in untreated cells.

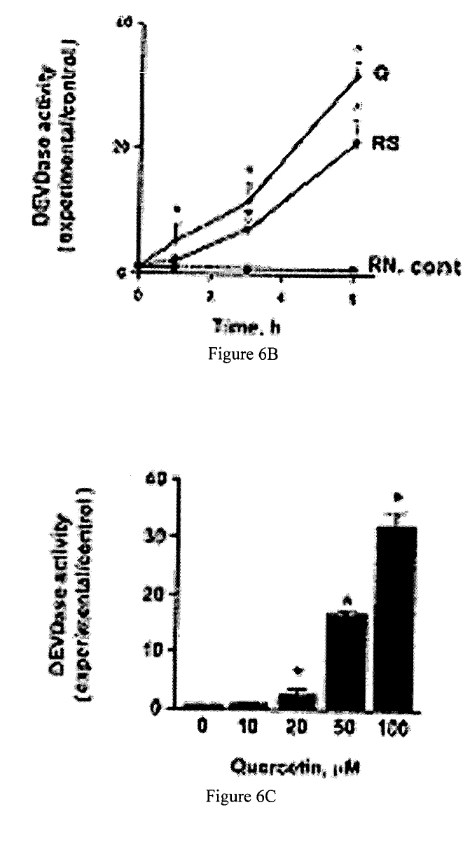

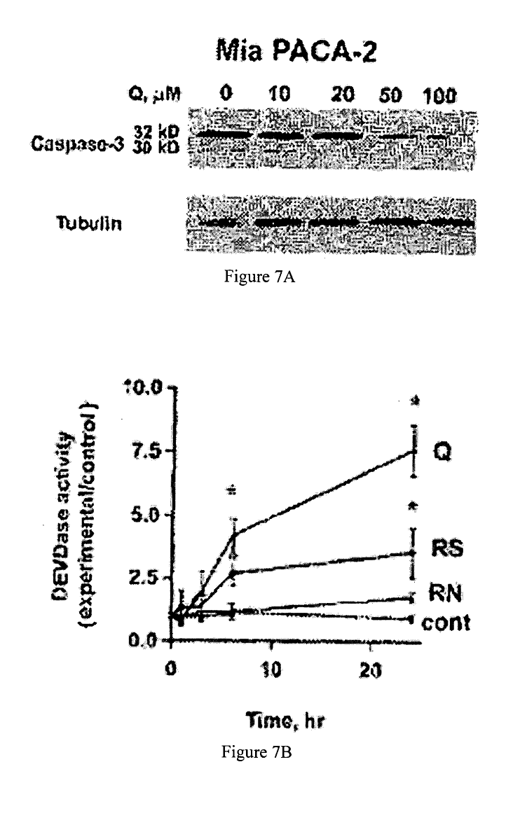

As provided in FIGS. 6A and 7A, both quercetin and trans-resveratrol convert caspase-3 from its inactive form (32 kDa doublet) to its active form (17 kDa) as illustrated by a decrease in the inactive form and an increase in the active form using Western blot analysis and an antibody that recognizes both forms. The results show a dose dependency with effects occurring with as little as 20 μM for both compounds. FIGS. 6B, 6C, and 7B show that both quercetin and trans-resveratrol, but not rutin, caused caspase-3 activation as provided in the specific fluorogenic assay for caspase-3. As shown in FIGS. 6A, 6C, and 7A, the effects on caspase-3 activity were dose dependent and as shown in FIGS. 6B and 7B, the effects on caspase-3 activity were time dependent. In particular, the results show that effects of quercetin and trans-resveratrol on apoptosis were more rapid in BSp73AS cells as compared to Mia PACA-2 cells.

II. Combinations

To determine the role of caspases in the effects of combinations of agents on apoptosis, the effects of caspase-3 activity (DEVDase activity) as well as the effect of the broad spectrum caspase inhibitor, Z-VAD, on apoptosis was measured. Specifically, Mia PACA-2 cells were cultured for 72 hours in the presence of serum with or without 100 μM Z-VAD, 15 μM DPI, 100 μM trans-resveratrol, 100 μM genistein, a combination of trans-resveratrol and DPI, and a combination of genistein and DPI. DEVDase activity was measured in whole cell lysates with a fluorimetric assay. DNA fragmentation was measured in cell lysates by cell death ELISA.

The ELISA assay for DNA fragmentation was conducted as described above. A fluorimetric assay for caspase-3 activity was conducted. Specifically, cells were collected, washed with ice-cold PBS and resuspended in lysis buffer comprising 0.5% Nonidet P-40 or manufactured by the name IGEPAL CA-630, 0.5 mM EDTA, 150 mM NaCl and 50 mM Tris at pH 7.5. Cell lysates were placed for 30 minutes on a rotator at 4° C. and then centrifuged for 15 minutes at 15,000 g. Cytosolic protein extracts (supernatants) were collected, protein concentrations were determined and the extracts were aliquoted and stored at −80° C. Enzyme assays were carried out at 37° C. in a buffer comprising 25 mM HEPES (pH 7.5), 10% sucrose, 0.1% CHAPS and 10 mM DTT with 800 g cytosolic protein and 20 μM of specific fluorogenic substrate. For capsase-3, the substrate was z-DEVD. Cleavage of the caspase substrate releases 7-amino-4-methylcoumarin (AMC), which emits a fluorescent signal with excitation at 38 nm and emission at 440 nm. The reaction was started by addition of caspase-3 substrate, the readings were taken at 0, 60, 90, and 120 minutes. Fluorescence was calibrated using a standard curve for AMC. The data were expressed as mol AMC/mg protein/min.

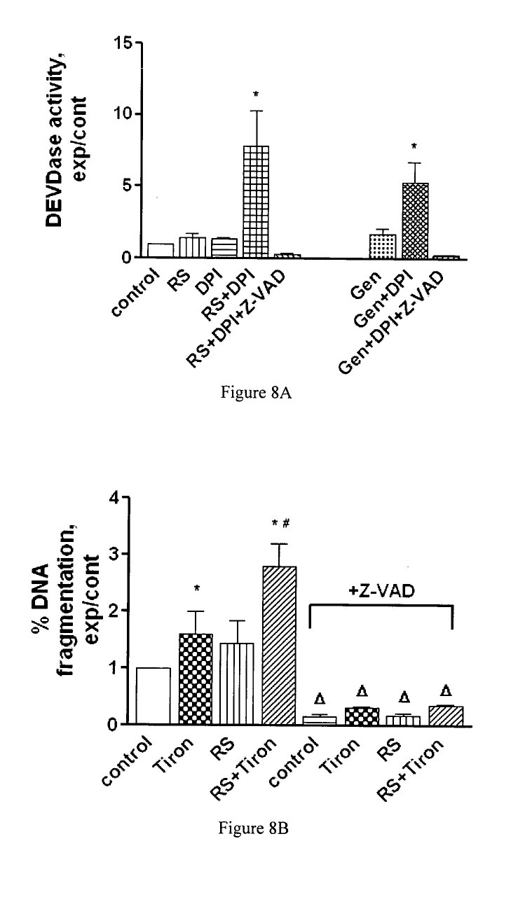

As shown in FIG. 8, the caspase-3 activity and apoptosis are synergistically activated with a combination of an inhibitor of ROS production and a polyphenolic compound and Z-VAD inhibits apoptosis caused by the combinations.

### EXAMPLE 5

### Mitochondrial Assays

I. Polyphenolic Compound Alone

In order to determine the effects polyphenolic compounds have on mitochondrial cytochrome c release and apoptosis, the following assay was conducted. BSp73AS cells were cultured for 6 hours and Mia PACA-2 cells were cultured in the absence of serum or growth factors for 24 hours in the absence or presence of 100 μM of each rutin, trans-resveratrol, genistein, or quercetin. Cells were washed twice with ice-cold PBS, pH 7.2 and resuspended in extraction buffer, about 500 μl, comprising 20 mM HEPES-KOH (pH 7.0), 10 mM KCl, 1 mM NaEGTA, 2 mM MgCl₂, 1 mM EDTA, 1 mM DTT, 250 mM sucrose, 1 mM PMSF and protease inhibitors cocktail as provided above. The lysate was incubated for 30 minutes on ice and then homogenized using a glass dounce (80 strokes). Nuclei were removed by centrifugation at 1,000 g for 10 minutes at 4° C. Supernatant was additionally centrifuged for 1 hour at 100,000 g and the resulting supernatant (cytosolic fraction) and pellet (mitochondrial fraction) were collected separately, subjected to SDS-PAGE, and Western blotting using an antibody against cytochrome c. The experiment was repeated twice with similar results.

In order to determine the effects polyphenolic compounds have on mitochondrial membrane potential, the retention of the dye 3,3′-dihexyloxacarbocyanine (DiOC₆(3)) was measured as described by Pastorino J G, et al. (1998) J Biol Chem 273:7770-7775, which is herein incorporated by reference. BSp73AS cells were cultured for 6 hours in the absence (control) or presence of 0, 12, 24, 50, and 100 μM quercetin and trans-resveratrol. The cells were loaded with 1 μM DiOC₆(3) during the last 30 minutes of treatment with a polyphenolic compound (or vehicle). The cells were then collected and pelleted by centrifugation. The supernatant was removed and the pellet was washed twice with PBS by alternate centrifugation and resuspension. The pellet was then lysed by addition of 1 ml of H₂O and homogenized. The concentration of DiOC₆(3) was read on a Perkin-Elmer LS-5 fluorescence spectrometer at 488 nm excitation and 500 nm emission. An aliquot of the cells was used for determining the DiOC₆(3) fluorescence that was retained by the cells.

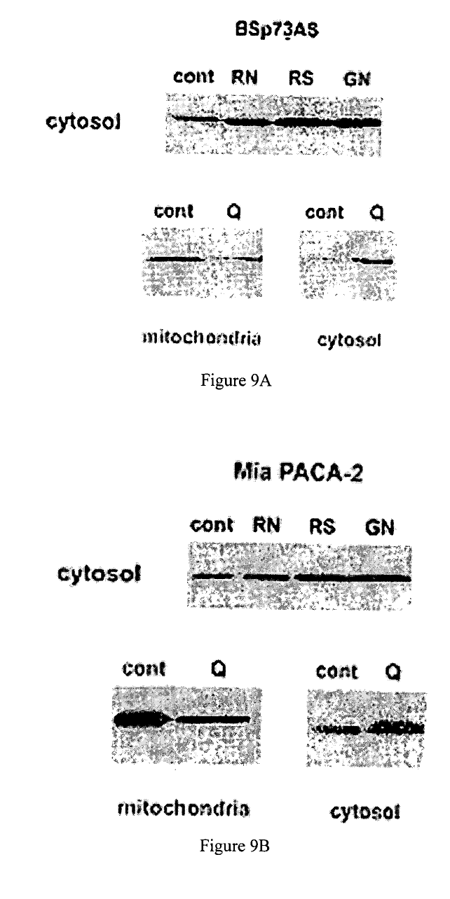

As shown in FIG. 9, quercetin, trans-resveratrol and genistein, but not rutin, caused increases in cytosolic cytochrome c and decreases in mitochondrial cytochrome c

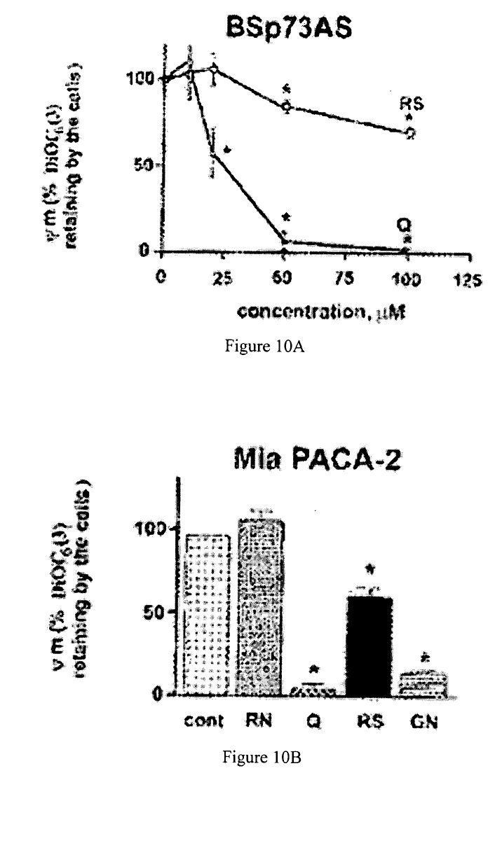

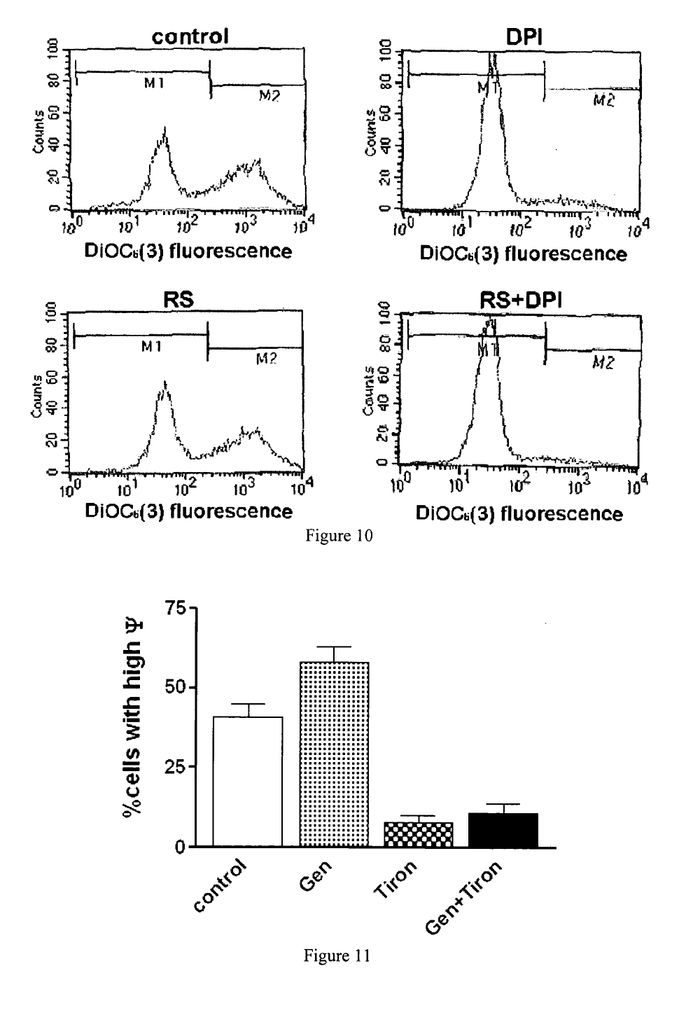

As shown in FIG. 10, quercetin, trans-resveratrol, and genistein, but not rutin, caused dissipation of the mitochondrial membrane potential using a dye, DiOC₆(3), that is taken up in cells as a function of mitochondrial membrane potential. Because one mechanism of mitochondrial cytochrome c release involves opening of the mitochondrial permeability transition pore (PTP), which is associated with dissipation of the mitochondrial membrane potential, the results as shown in FIGS. 9 and 10 suggest that the mechanism of action of the polyphenolic compounds on cytochrome c release and apoptosis is through the PTP.

II. Combinations

In order to determine the effects of the polyphenolic compounds alone and in combination with ROS inhibitors on mitochondrial membrane potential in the presence of serum, the following assay was performed.

Mia PACA-2 cells were cultured for 72 hours in the presence of serum and trans-resveratrol or genistein in the presence or absence of DPI or Tiron. Mitochondrial membrane potential was measured as described above.

As shown in FIG. 11, trans-resveratrol alone and genistein alone have no effects on mitochondrial membrane potential. Additionally, DPI and Tiron markedly depolarized the mitochondrial membrane and the depolarization was not changed by the addition of trans-resveratrol or genistein. Thus, in the presence of serum, the production of ROS is essential for the maintenance of mitochondrial membrane polarity and the ability of polyphenolic compounds to potentiate the effects of ROS inhibitors on apoptosis is not due to changes in membrane polarity.

### EXAMPLE 6

### Effects of Inhibitors of PTP

A. Polyphenolic Compound Alone

In order to determine the role of the cancer cell's PTP in the effects of the polyphenolic compounds on mitochondrial function, caspase-3 activation and apoptosis, we performed the following experiments. Mia PACA-2 cells were cultured in the absence of serum or growth factors for 24 hours in the presence or absence of quercetin (100 μM), trans-resveratrol (100 μM), Z-VAD FMK (50 μM), cyclosporin A (5 μM) and/or aristolochic acid (50 μM). Cells were collected, washed with ice-cold PBS and resuspended in lysis buffer comprising 0.5% Nonidet P-40 or manufactured by the name IGEPAL CA-630, 0.5 mM EDTA, 150 mM NaCl and 50 mM Tris at pH 7.5. Cell lysates were placed for 30 minutes on a rotator at 4° C. and then centrifuged for 15 minutes at 15,000 g. Cytosolic protein extracts (supernatants) were collected, protein concentrations were determined and the extracts were aliquoted and stored at −80° C. Cytosolic extracts were subjected to SDS-PAGE and Western blot was performed with an antibody against cytochrome c. Blots were then stripped and re-probed with an antibody against tubulin to confirm equal protein loading. The experiment was repeated twice with similar results.

Mia PACA-2 cells were cultured in the absence of serum or growth factors for 24 hours in the presence or absence of quercetin (100 μM), trans-resveratrol (100 μM), Z-VAD FMK (50 μM), cyclosporin A (5 μM) and/or aristolochic acid (50 μM). Enzyme assays were carried out at 37° C. in a buffer comprising 25 mM HEPES (pH 7.5), 10% sucrose, 0.1% CHAPS and 10 mM DTT with 800 g cytosolic protein and 20 μM of specific fluorogenic substrate. For caspase-3, the substrate was K-AspGluValAsp-AMC (z-DEVD). Cleavage of the caspase substrate releases AMC (7-amino-4-methylcoumarin), which emits a fluorescent signal with excitation at 380 nm and emission at 440 nm. The reaction was started by addition of caspase-3 substrate, the readings were taken at 0, 60, 90, and 120 minutes. Fluorescence was calibrated using a standard curve for AMC. The results were normalized to the DEVDase activity in cells not treated with polyphenolic compounds. The data were expressed as mol AMC/mg protein/min.

Mia PACA-2 cells were cultured in the absence of serum or growth factors for 24 hours in the presence or absence of quercetin (100 μM), trans-resveratrol (100 μM), Z-VAD FMK (50 μM), cyclosporin A (5 μM) and aristolochic acid (50 μM). The samples were analyzed by annexin staining as provided above.

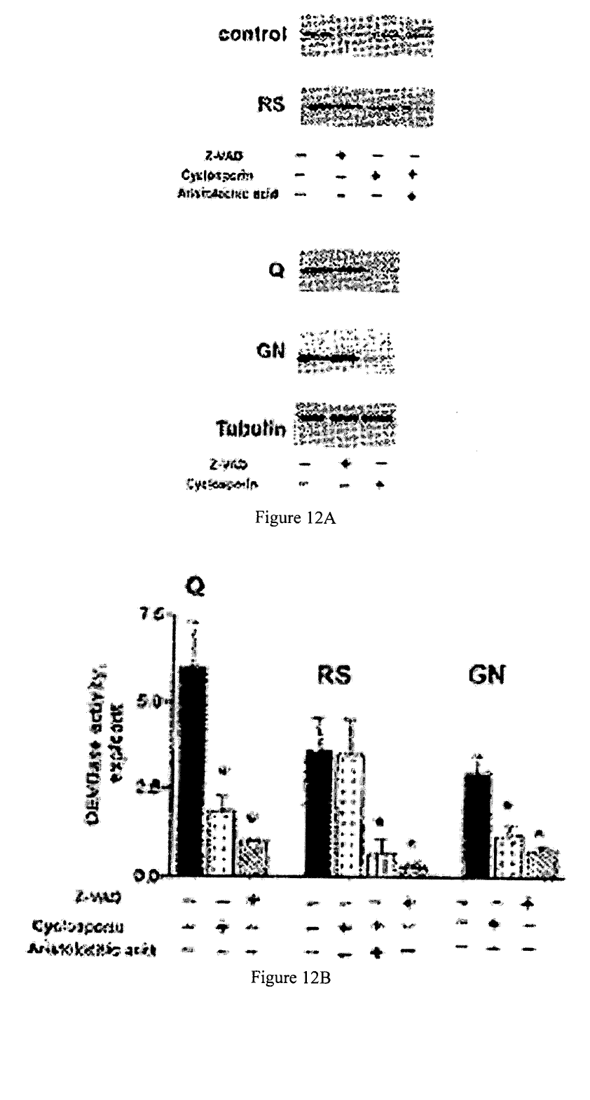

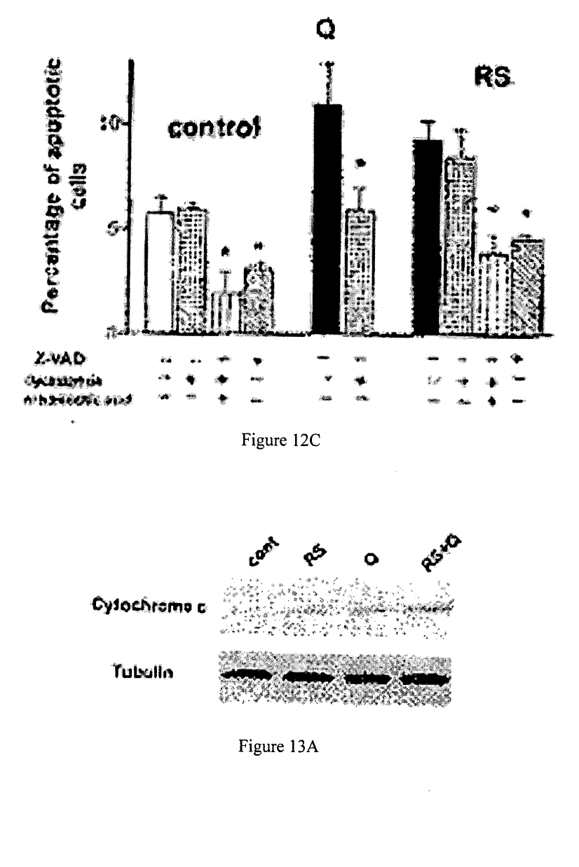

Cyclosporin A inhibits PTP channels by interacting with one of the key subunits of the PTP, cyclophilin, and cyclosporin A by itself or in combination with aristolochic acid blocks cytochrome c release on several cell types. As illustrated in FIG. 12A, Z-VAD inhibited the release of cytochrome c into the cytoplasm in control (untreated) cells. In contrast, Z-VAD had no effect on cytosolic cytochrome c release caused by quercetin or trans-resveratrol, thereby indicating that their action is directly on the mitochondria and not through the pathway involving caspase-8 and Bid. The cytochrome c release caused by quercetin and genistein were inhibited by cyclosporin A alone, whereas the cytochrome c release caused by trans-resveratrol required both cyclosporin A and aristolochic acid for inhibition.

As shown in FIG. 12B, cyclosporin A alone inhibited caspase-3 activity in quercetin and genistein treated cells, whereas both cyclosporin A and aristolochic acid were required to inhibit caspase-3 activity in trans-resveratrol-treated cells. As shown in FIG. 12C, cyclosporin A alone inhibited apoptosis in quercetin-treated cells, whereas both cyclosporin A and aristolochic acid were required to inhibit apoptosis caused by trans-resveratrol. These results indicate that polyphenolic compounds cause apoptosis from their direct effects on the cancer cell mitochondrial PTP to cause cytochrome c release which, in turn, activates caspase-3 leading to apoptosis.

B. Combination

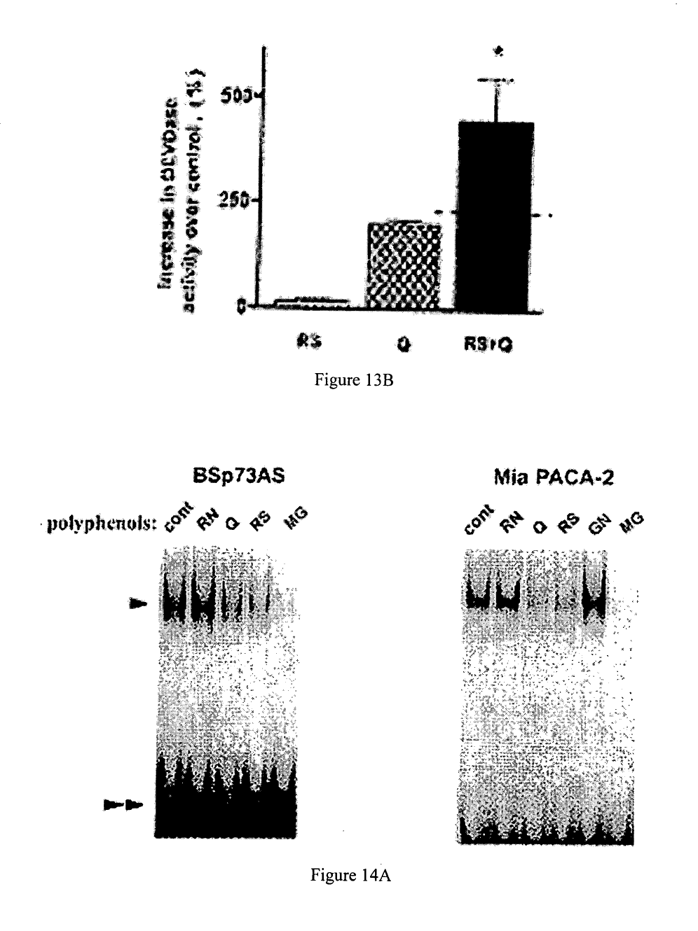

In order to determine the effect of a combination of trans-resveratrol and quercetin on cytochrome c release and caspase-3 activity, the following was conducted. Mia PACA-2 cells were cultured in the absence of serum or growth factors for 24 hours in the presence or absence of trans-resveratrol (25 μM), quercetin (25 μM), or the combination of trans-resveratrol (25 μM) and quercetin (25 μM). Cytosolic extracts were prepared and subject to SDS-PAGE followed by protein transfer. Immunoblot was performed with an antibody against tubulin to confirm equal protein loading. Mia PACA-2 cells were cultured in the absence of serum or growth factors for 24 hours in the presence or absence of trans-resveratrol (25 μM), quercetin (25 μM), or the combination of trans-resveratrol (25 μM) and quercetin (25 μM). Caspase-3 activity was measured in cell lysates with a fluorgenic assay using DEVD-AMC as a substrate. The results were normalized to the DEVDase activity in untreated cells. As illustrated in FIG. 13, the combinations resulted in responses of cytochrome c release and caspase-3 activity that were significantly greater than the additive responses.

### EXAMPLE 7

### NF-κB Assays

I. Polyphenolic Compound Alone

In order to determine the role of activated NF-κB in the regulation of apoptosis caused by the polyphenolic compound, the following assay was conducted. BSp73AS cells were cultured for 6 hours and Mia PACA-2 cells were cultured in the absence of serum or growth factors for 24 hours in the absence (controls) or presence of rutin, quercetin, trans-resveratrol, or genistein, each at 100 μM and 20 μM proteosome inhibitor MG-132. Nuclear proteins were isolated and analyzed for NF-κB DNA binding activity with electrophoretic mobility shift assay (EMSA).

Specifically, aliquots of nuclear extracts with equal amount of protein, about 2 to about 10 μg, were mixed in 20 μl reactions with a buffer comprising 10 mM HEPES (pH 7.6), 50 mM KCl, 0.1 mM EDTA, 1 mM DTT, 10% (vol/vol) glycerol and 3 μg poly[d (I-C)]. After aliquots were equilibrated on ice for 5 minutes, binding reactions were stared by addition of 20-60,000 counts/min (20 μM) of ³²P-labeled DNA probes and allowed to proceed for 25-30 minutes at room temperature or up to 1 hour on ice. The oligonucleotide probe

| | |
|---|---|
| 5′-GCAGAGGGGACTTTCCGAGA | (SEQ ID NO:5) |

containing the κB binding motif (underlined) was annealed to the complementary oligonucleotide with a 5′-G overhang and end-labeled using Klenow DNA polymerase I. The samples were electrophoresed at room temperature in 0.5×TBE buffer (1×TBE 89 mM Tris base, 89 mM boric acid and 2 mM EDTA) on nondenaturing 4.5% polyacrylamide gel at 200 V. Gels were dried and directly analyzed in the Phosphorlmager (Molecular Dynamics, Sunnyvale, Calif.). The experiment was repeated twice.

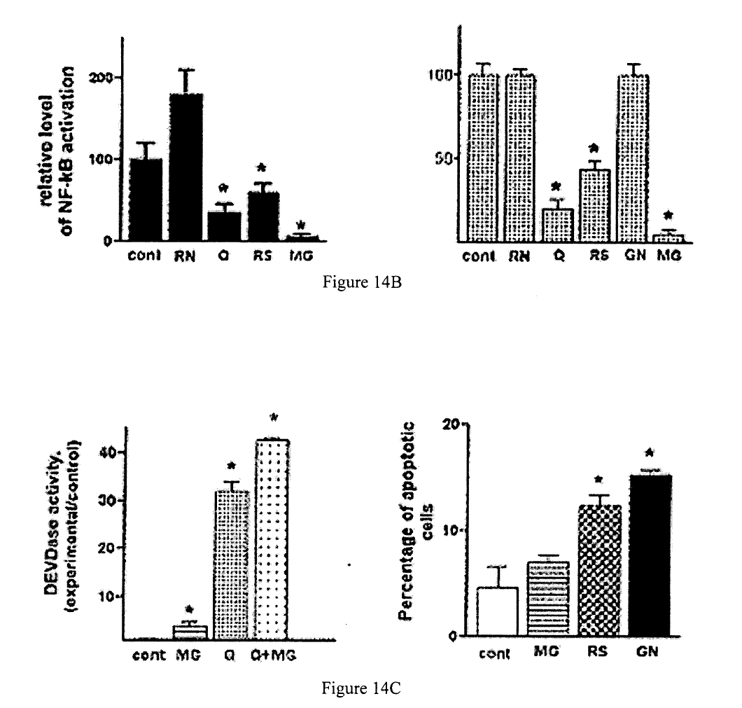

As shown in FIG. 14A, NF-κB is constitutively active in both cancer cell lines. FIGS. 14A and 14B show that quercetin and trans-resveratrol inhibit NF-κB activation in both pancreatic cell lines, rutin activates NF-κB in BSp73AS cells but not Mia PACA-2 cells, and genistein has no effect on NF-κB in the Mia PACA-2 cells. The proteosome inhibitor, MG-132, blocks NF-κB activation in both cell lines. As shown in FIG. 14C, MG-132 causes a small increase in caspase-3 activity that adds to the caspase-3 activity caused by quercetin. Complete inhibition of NF-κB by MG-132 does not increase apoptosis rates to the same extent as trans-resveratrol which only partially inhibits NF-κB activation. Additionally, genistein causes significant apoptosis in the absence of an effect on NF-κB activation.

II. Combinations

To study the effects of combinations of DPI and polyphenolic compounds on serum-induced activation of NF-κB and protection from apoptosis, the following assay was conducted. Mia PACA-2 cells were cultured for 72 hours in the presence of serum with or without 100 μM trans-resveratrol, 15 μM DPI, a combination of trans-resveratrol, 100 μM genistein, or a combination of genistein and DPI. NF-κB binding activity was measured in nuclear extracts by gel shift assay as described above.

In order to demonstrate the cause and effect relationship between serum-induced activation of NF-κB and protection from apoptosis, NF-κB activation was inhibited and the effects on apoptosis in the presence and absence of polyphenolic compounds and ROS inhibitors were studied. In particular, Mia PACA-2 cells were cultured for 72 hours in the presence of serum with or without 50 μM trans-resveratrol (RS), 15 μM DPI, a combination of DPI and trans-resveratrol (RS+DPI), 10 μM MG-132 alone and in combination with RS, DPI, and RS+DPI. Internucleosomal DNA fragmentation was measured in cell lysates by cell death ELISA.

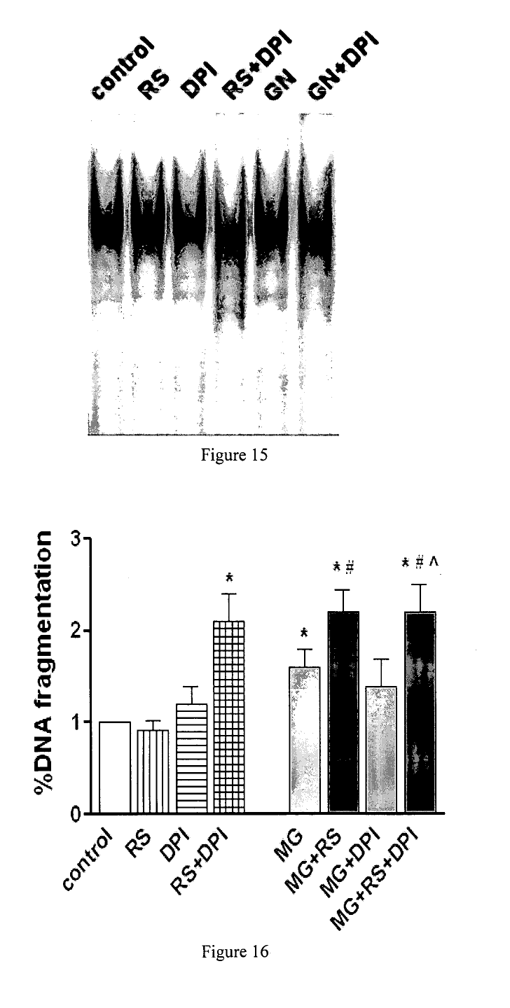

As shown in FIG. 15, the combination of a polyphenolic compound and a ROS inhibitor prevents NF-κB activation caused by serum. As shown in FIG. 16, trans-resveratrol in combination with MG-132 alone or MG-132 plus DPI increased apoptosis to a greater degree than that observed with MG-132 alone or MG-132 plus DPI, thereby indicating that inhibition of NF-κB sensitizes the cancer cells to apoptosis caused by trans-resveratrol.

III. Peptide Inhibitors of NF-κB

A. Reagents

CCK-8 was from American Peptide Company (Sunnyvale, Calif.); recombinant TNF-α, from BD Biosciences (San Diego, Calif.); medium 199, from GIBCO BRL (Grand Island, N.Y.); [γ-³²P]ATP, from ICN Biomedicals (Costa Mesa, Calif.); GF-109203×, Gö6976, PKC δ peptide substrate, PKC α peptide substrate, from Calbiochem (La Jolla, Calif.); PKC ζ substrate and PKC ε pseudosubstrate, from Biosource International (Camarillo, Calif.); antibodies against IκBα, PKC α, PKC ε, PKC δ, and PKC ζ, from Santa Cruz Biotechnology (Santa Cruz, Calif.); PP2, D-609, U-73122, from Biomol (Plymouth Meeting, Pa.); conventional PKC substrate and anti-phosphotyrosine antibody, from Upstate Biotechnology (Charlottesville, Va.); T4 polynucleotide kinase, from New England BioLabs (Beverly, Mass.); and poly(dI-dC), from Boehringer Mannheim (Indianapolis, Ind.). All other chemicals were from Sigma Chemical (St. Louis, Mo.).

B. Preparation of Dispersed Pancreatic Acini

Pancreatic acini were prepared from Sprague-Dawley rats (about 75 to about 100 g) using a collagenase digestion method known in the art and then incubated in 199 medium supplemented with penicillin (100 U/ml) and streptomycin (0.1 mg/ml) for 3 hours at 37° C. in a 5% CO₂ humidified atmosphere. These incubation conditions are referred to herein as “standard incubation conditions”.

C. Preparation of Nuclear Extracts and Electrophoretic Mobility Shift Assay

Preparation of nuclear and cytosolic protein extracts, and the electrophoretic mobility shift assay (EMSA) were conducted according to methods known in the art. Briefly, pancreatic acinar cells were lysed on ice in a hypotonic buffer A supplemented with 1 mM PMSF, 1 mM DTT, and protease inhibitor cocktail containing 5 μg/ml each of pepstatin, leupeptin, chymostatin, antipain and aprotinin. Cells were left to swell on ice for about 20 to about 25 minutes, then 0.3% (vol/vol) Igepal CA-630 was added, and the nuclei were collected by microcentrifugation. The supernatant (cytosolic proteins) was saved for Western blot analysis of IκBα, and the nuclear pellet resuspended in a high-salt buffer C supplemented with 1 mM PMSF, 1 mM DTT, and the protease inhibitor cocktail described above. After incubating at 4° C., membrane debris were pelleted by microcentrifugation for 10 minutes, and the clear supernatant (nuclear extract) was aliquoted and stored at −80° C. Protein concentration in the extracts was determined by the Bio-Rad protein assay (Bio-Rad Laboratories, Hercules, Calif.).

For EMSA, aliquots of nuclear extracts with equal amounts of protein (about 5 to about 10 μg) were mixed in 20 μl reactions with a buffer containing 10 mM HEPES (pH 7.8), 50 mM KCl, 0.1 mM EDTA, 1 mM DTT, 10% glycerol, and 3 μg poly(dI-dC). Binding reactions were started by addition of ³²P-labeled DNA probe and incubated at room temperature for 20 minutes. The oligo probe

| | |
|---|---|
| 5′-GCAGAGGGGACTTTCCGAGA | (SEQ ID NO: 5) |

containing κB binding motif (underlined) was annealed to the complementary oligonucleotide and end-labeled using T4 polynucleotide kinase. Samples were electrophoresed on a native 4.5% polyacrylamide gel at 200 V in 0.5×TBE buffer (1×TBE: 89 mM Tris base, 89 mM boric acid, 2 mM EDTA). Gels were dried and densitometrically quantified in the Phosphorlmager (Molecular Dynamics, Sunnyvale, Calif.). In pancreatic acinar cells, the NF-κB band has 2 components: the upper component corresponds to the p50/p65 heterodimer and the lower component to the p50/p50 homodimer. See Pandol et al. (1999) Gastroenterology 117:706-716, which is herein incorporated by reference. In the present study, the total (combined) intensity of the NF-κB band was quantified.

D. Subcellular Fractionation

The dispersed acini were homogenized with 50 strokes in a Dounce homoginizer in an ice-cold homogenization buffer containing 130 mM NaCl, 50 mM Tris/HCl (pH 7.5), 5 mM EGTA, 5 mM EDTA, 1.5 mM MgCl2, 10 mM NaF, 1 mM Na3VO4, 10 mM Na4P207, 10% (vol/vol) glycerol, 1 mM PMSF, and 5 μg/ml each of pepstatin, leupeptin, chymostatin, antipain, and aprotinin. Homogenates were centrifuged at 500 g for 10 minutes at 4° C. to remove unbroken cells, nuclei, and other debris. Supernatants were recovered and ultracentrifuged at 150,000 g for 45 minutes at 4° C. to separate the cytosolic fraction (the resulting supernatant) and the pellet for translocation experiments. The pellet was washed 5 times with homogenization buffer, resuspended in a homogenization buffer containing 2% (vol/vol) Triton X-100, sonicated 5 times for 10 sec on ice, and incubated for 30 minutes at 4° C. At the end of incubation, the samples were centrifuged at 15,000 g for 15 minutes, and the resulting supernatant was designated the membrane fraction.

E. Immunoprecipitation

Pancreatic acini were suspended in 1 ml of ice-cold homogenization buffer, sonicated 5 times for 10 seconds on ice, and incubated for 45 minutes at 4° C. After the centrifugation for 15 minutes at 15,000 g, specific antibody against individual PKC (Santa Cruz, Santa Cruz, Calif.) isoform (1:100 dilution) was added to the lysate, which was rotated overnight at 4° C. Protein A-Sepharose beads (50% slurry) were added and rotated for 2 hours at 4° C. The beads were washed twice in the lysis buffer followed by additional 3 washes with the kinase buffer (20 mM MOPS (pH 7.2), 25 mM β-glycerophosphate, 5 mM EGTA, 1 mM Na₃VO₄, and 1 mM DTT). The beads were resuspended in a final 50 μl of kinase buffer.

F. Isoform Specific PKC Kinase Assay

The kinase assay was performed using the PKC assay kit (Upstate Biotechnology, Charlottesville, Va.) according to the manufacturer's instruction with minor modifications. Substrates optimized for individual PKC isoforms were used. The substrates used are as follows:

| | | |
|---|---|---|
| For PKC α: | QKRPSQRSKYL | (SEQ ID NO:6) |
| For PKC δ: | RFAVRDMRQTVAVGVIKAVDKK | (SEQ ID NO:7) |
| For PKC ε: | ERMRPRKRQGSVRRRV | (SEQ ID NO:8) |
| For PKC ζ: | SIYRRGSRRWRKL. | (SEQ ID NO:9) |

The kinase buffer as provided in Section E above was used for the measurement of PKC δ, ε, and ζ, and supplemented it with 1 mM CaCl₂ for PKC α. The assay was started with the addition of a magnesium/ATP mixture (75 mM MgCl₂ and 0.5 mM ATP) containing 10 μCi of [y-³²P]ATP to the sample containing 10 μl of the PKC isoform specific immunoprecipitate, 30 μl of kinase buffer and 40 μM of substrate, and the reaction incubated for 10 minutes at 30° C. Reactions were stopped by the addition of 50 μl of 0.75% phosphoric acid, and the samples were applied onto p81 phosphocellulose paper (Upstate Biotechnology, Charlottesville, Va.). The p81 papers were washed three times with 0.75% phosphoric acid, once with acetone, and the amount of ³²P was determined by liquid scintillation counting known in the art. Background measurements of ³²P were determined from incubations conducted in the absence of substrate, and were subtracted from the ³²P values in experimental samples. Measurements were performed in duplication.

G. Western Blot Analysis

Immunoprecipitate of PKC δ for tyrosine phosphorylation analysis, cytosolic extracts for IκBα analysis, or subcellular fractions for PKC translocation studies were used as samples for Western blot analysis. After samples were adjusted for protein concentration, equal amounts of protein were fractionated by sodium dodecyl sulfate polyacrylamide gel electrophoresis (SDS-PAGE), and electrophoretically transferred to nitrocellulose membranes. The membranes were blocked by overnight incubation in Tris-buffered saline (TBS) supplemented with 5% nonfat dry milk and probed with an antibody against IκBα (1:100 dilution), PKC a, PKC δ, PKC ε, PKC ζ (1:200 dilution each), or phosphotyrosine (1:500 dilution) for 2 hours at room temperature. The membranes were incubated with secondary antibodies conjugated with horseradish peroxidase for 1 hour at room temperature. Blots were developed using the enhanced chemiluminescence detection kit (Pierce, Rockford, Ill.). When reprobing was necessary, the membrane was stripped of bound antibody by incubating in stripping buffer at room temperature for 20 minutes.

H. Inhibition Analysis

For pharmacologic analysis, we used a broad spectrum PKC inhibitor, GF109203X (GF); a specific inhibitor of conventional PKC isoforms, Gö6976 (Gö); and a specific PKC ζ inhibitor, PKC ζ pseudosubstrate. A specific PKC δ translocation inhibitor

| | |
|---|---|
| δV1-1:SFNSYELGSL | (SEQ ID NO:1) |

PKC ε translocation inhibitor

| | |
|---|---|
| εV1-2:EAVSLKPT | (SEQ ID NO:2) |

and scrambled peptide

| | |
|---|---|
| LSETKPAV | (SEQ ID NO:3) |

according to the art. For each of the PKC isoform, these peptides correspond to specific sequences in the V1 regions, which is responsible for anchoring the individual isoform to its translocation site. Thus, the peptides competitively inhibit the binding of a specific isoform of PKC to its anchoring protein. Each of these peptides was conjugated to a Drosophila antennapedia peptide

| | |
|---|---|
| RQIKIWFQNRRMKWKK | (SEQ ID NO:4) |

to make them cell-permeable.

The complete sequences as conjugated are as follows:

| | |
|---|---|
| SFNSYELGSLRQIKIWFQNRRMKWKK | (SEQ ID NO:10) |
| EAVSLKPTRQIKIWFQNRRMKWKK | (SEQ ID NO:11) |
| LSETKPAVRQIKIWFQNRRMKWKK | (SEQ ID NO:12) |

I. Assays

1. Subcellular Distribution and Kinase Activities of PKC Isoforms in Pancreatic Acini Stimulated by CCK-8

Dispersed rat pancreatic acini were preincubated in standard incubation conditions with 1% DMSO (vol/vol) for 3 hours, and then stimulated with 100 nM CCK-8 for 30 minutes. The scrambled peptide (10 μM) was used as a control and inhibitors and their controls were delivered in DMSO so that final DMSO concentration was 1% (vol/vol) in this a subsequent experiments. At the end of this incubation, subcellular fractions were obtained as described in Section D above and used for Western blot analysis as described in Section G above. Kinase activity measurements were performed on samples that were immunoprecipitated as described in Section E above with a specific antibody for the isoform of PKC to be measured. The kinase activity measurement was as described in Section F above.

FIG. 19A shows subcellular distribution of PKC isoforms in response to CCK-8 in cytosolic and membrane fractions using isoform specific PKC antibodies and Western blot analysis.

FIG. 19B shows the changes in PKC kinase activities stimulated by CCK-8. Individual PKC isoforms were immunoprecipitated from whole cell lysates and PKC activities were measured by kinase assay using isoform-optimized substrates.

2. Effects of PKC Inhibitors on CCK-8-induced NF-κB Activation in Pancreatic Acini

Pancreatic acini were preincubated for 3 hours with a PKC broad spectrum inhibitor, GF109203X (GF); a conventional PKC isoform inhibitor, Gö6976 (Gö); a PKC δ translocation inhibitor (δV1-1); a PKC ε translocation inhibitor (εV1-2); a PKC ζ inhibitor, a PKC ζ pseudosubstrate (ζ pseudo), 10 μM each, or with DMSO, and then stimulated with 100 nM CCK-8 for 30 minutes. At the end of this incubation nuclear extracts were prepared and subjected to electomobility shift assay (EMSA) for NF-κB binding activity as described in Section C above. IκBα analysis was performed on cytosolic factions using Western blot analysis as described in Section G above.

FIG. 20A shows NF-κB binding activity measured in nuclear extracts measured by EMSA.

FIG. 20B shows NF-κB band intensities quantified in the PhosphorImager and normalized on the band intensity in unstimulated control acini.

3. Specificity of the Isoform-Specific Translocation Inhibitors

Pancreatic acini were preincubated with PKC translocation inhibitors, δV1-1 or εV1-2 (10 μM each), scrambled peptide (10 μM), delivered in DMSO for 3 hours, and then stimulated with 100 nM CCK-8 for 30 minutes. At the end of this incubation, subcellular fractions were obtained as described in Section D above and used for Western blot analysis as described in Section G above. Kinase activity measurements were performed on samples that were immunoprecipitated as described in Section E above with a specific antibody for the isoform of PKC to be measured. The kinase activity measurement was as described in Section F above.

FIG. 21A shows cytosolic and membrane fractions subjected to Western blot analysis. SDS-PAGE and blotted using antibodies specific for PKC δ or ε were used.

FIG. 21B shows the effects of PKC translocation inhibitors on kinase activity. For each PKC isoform, activity values were normalized on its basal activity in unstimulated control acini.

4. Subcellular Distribution and Kinase Activities of PKC Isoforms in Pancreatic Acini Stimulated by TNF-α

Pancreatic acini were preincubated for 3 hours with in standard incubation conditions with 1% DMSO (vol/vol) for 3 hours, and then stimulated with 100 ng/ml TNF-α for 30 minutes. The scrambled peptide (10 μM) was used as a control and inhibitors and their controls were delivered in DMSO so that final DMSO concentration was 1% (vol/vol) in this a subsequent experiments. At the end of this incubation, subcellular fractions were obtained as described in Section D above and used for Western blot analysis as described in Section G above. Kinase activity measurements were performed on samples that were immunoprecipitated as described in Section E above with a specific antibody for the isoform of PKC to be measured. The kinase activity measurement was as described in Section F above.

FIG. 22A shows subcellular distribution of PKC isoforms in response to TNF-α in cytosolic and membrane fractions using isoform specific PKC antibodies and Western blot analysis.

FIG. 22B shows changes in PKC kinase activities stimulated by TNF-α. Individual PKC isoforms were immunoprecipitated from whole cell lysates and PKC activities were measured by kinase assay using isoform-optimized substrates. For each PKC isoform, activity values were normalized on its basal activity in unstimulated control acini.

5. Effects of PKC Inhibitors on TNF-α Induced NF-κB Activation in Pancreatic Acini

Pancreatic acini were preincubated for 3 hours with PKC broad spectrum inhibitor, GF109203X (GF); conventional PKC isoform inhibitor, Gö6976 (Gö); PKC 6 translocation inhibitor (δV1-1); PKC ε translocation inhibitor (εV1-2); PKC ζ inhibitor, PKC ζ pseudosubstrate (ζ pseudo), 10 μM each, or with DMSO, and then stimulated with 100 ng/ml TNF-α for 30 minutes. At the end of this incubation nuclear extracts were prepared and subjected to electomobility shift assay (EMSA) for NF-κB binding activity as described in Section C above. IκBα analysis was performed on cytosolic factions using Western blot analysis as described in Section G above.

FIG. 23A shows NF-κB binding activity in nuclear extracts measured by EMSA.

FIG. 23B shows NF-κB band intensities quantified in the Phosphorlmager and normalized on the band intensity in unstimulated control acini.

FIG. 23C shows IκBα degradation in cytosolic extracts by Western blot analysis.

6. Effects of Src Kinase Inhibitor on NF-κB Activation Induced by CCK-8 and TNF-α

Pancreatic acini were preincubated with Src kinase inhibitor, PP2 (20 μM), or DMSO (vehicle) for 3 hours, and then stimulated with 100 nM CCK-8 or 100 ng/ml of TNF-α for 30 minutes. At the end of this incubation nuclear extracts were prepared and subjected to electomobility shift assay (EMSA) for NF-κB binding activity as described in Section C above. IκBα analysis was performed on cytosolic fractions as described in Section G above. IκBα analysis was performed on cytosolic factions using Western blot analysis as described in Section G above.

FIG. 24A shows NF-κB binding activity in nuclear extracts measured by EMSA.

FIG. 24B shows IκBα degradation in cytosolic extracts by Western blot analysis.

7. Effects of Src Kinase Inhibitor on Tyrosine Phosphorylation of PKC 6

Pancreatic acini were preincubated with with Src kinase inhibitor, PP2 (20 μM), or DMSO (vehicle) for 3 hours, and then stimulated with 100 nM CCK-8 or 100 ng/ml of TNF-α for 30 minutes. At the end of this incubation whole cell lysates were immunoprecipitated with an antibody specific to PKC δ as described in Section E above. The resulting immunoprecipitate was subjected to Western blot analysis with an antibody to phosphotyrosine as described in Section G above.

The upper panel of FIG. 25 shows whole cell lysates immunoprecipitated with anti-PKC δ antibody, and then subjected to SDS-PAGE and blotted using anti-phosphotyrosine antibody. The lower panel of FIG. 25 shows equal protein loading was verified using PKC δ antibody after stripping the membranes.

8. Effects of PLC Inhibitors on NF-κB Activation Induced by CCK-8 and TNF-α

Pancreatic acini were preincubated for 3 hours with PI-specific PLC inhibitor U-73122 (10 μM), or for 30 minutes with PC-specific PLC inhibitor D-609 (50 μM), and then stimulated for 30 minutes with 100 nM CCK-8 or 100 ng/ml TNF-α. D-609 was added to the culture medium 30 minutes before the stimulation because longer incubation with this inhibitor was toxic for pancreatic acini. At the end of this incubation nuclear extracts were prepared and subjected to electomobility shift assay (EMSA) for NF-κB binding activity as described in Section C above. IκBα analysis was performed on cytosolic fa ctions as described in Section G above.

FIG. 26A shows NF-κB binding activity in nuclear extracts measured by EMSA.

FIG. 26B shows IκBα degradation in cytosolic extracts measured by Western blot analysis.

9. Effects of PLC Inhibitors on PKC Translocation Induced by CCK-8 and TNF-α

Pancreatic acini were preincubated for 3 hours with PI-specific PLC inhibitor U-73122 (10 μM), or for 30 minutes with PC-specific PLC inhibitor D-609 (50 μM), and then stimulated for 30 minutes with 100 nM CCK-8 (FIG. 27A) or 100 ng/ml TNF-α (FIG. 27B). At the end of this incubation, subcellular fractions were obtained as described in Section D above and used for Western blot analysis as described in Section G above.

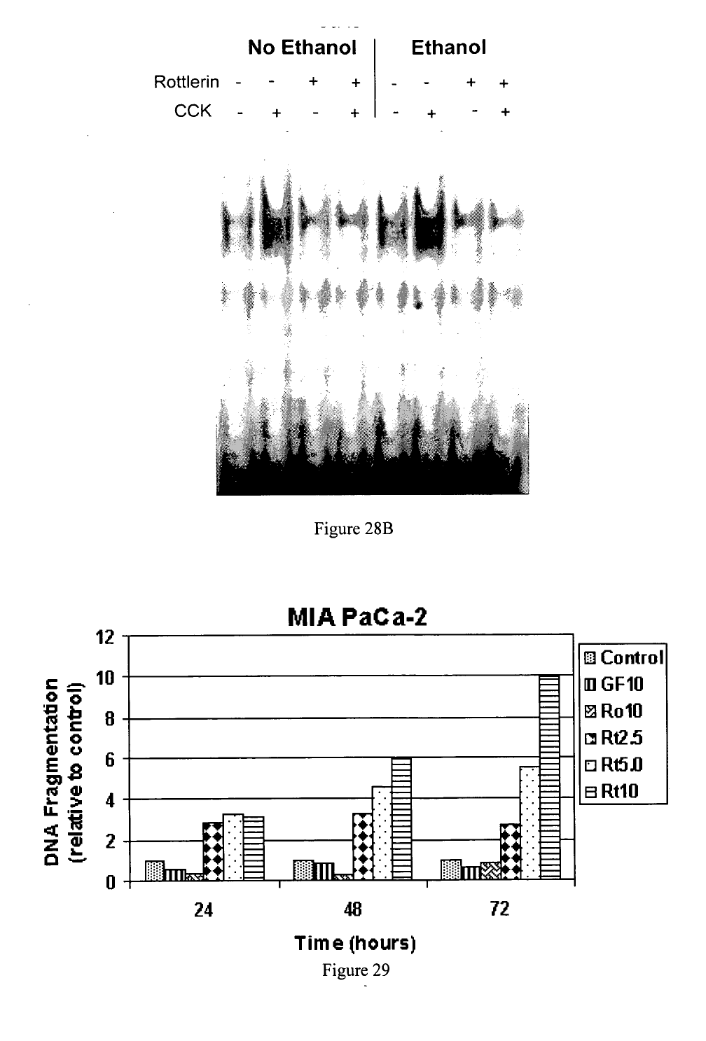

FIG. 28A is a schematic of the signaling pathways involved in NF-κB activation induced by CCK-8 and TNF-α in pancreatic acinar cells. Binding of CCK-8 to its receptor activates both PI-specific and PC-specific PLC, whereas TNF-α only activates PC-specific PLC. Activation of PLC leads to DAG generation, promotes translocation of PKC δ and PKC ε, which, in turn, mediates IκBa degradation and NF-κB activation. CCK-8 and TNF-α also induce PKC ζ activation, but it is not involved in NF-κB activation. Constitutive activity of PKC a exerts an inhibitory effect on NF-κB activation. Although tyrosine phosphorylation of PKC δ is induced by Src, this event is not involved in NF-κB activation induced by CCK-8 and TNF-α.

J. Rottlerin Inhibits NF-κB Activation in Pancreatic Acinar Cells

FIG. 28B was performed to test if a nonpeptide agent know to inhibit PKC 6 could inhibit NF-kB in pancreatic acinar cells. Pancreatic acini were preincubated for 3 hours with or without ethanol (100 mM) and with or without rottlerin (2.5 μM) for 30 minutes and then stimulated for 30 minutes with 100 nM CCK-8. At the end of this incubation nuclear extracts were prepared and subjected to electomobility shift assay (EMSA) for NF-κB binding activity as described in Section C above. The results show complete inhibition of NF-kB by rottlerin.

K. Statistical Analysis

The results as provided in the figures are expressed as means±SE. The percent changes in NF-κB activation and PKC activity were calculated as the difference between stimulated and unstimulated (basal) conditions. Statistics were performed using a paired t-test known in the art. A difference with a p-value of <0.05 was considered statistically significant.

### EXAMPLE 8

### PI 3-Kinase Inhibition Assays

To determine whether phosphatidylinositol 3-kinase (PI 3-kinase) and Akt/PKB mediate the effects of serum on NF-κB activation and that the effects of polyphenolic compounds on NF-κB activation are due to an ability to inhibit PI 3-kinase, the following assays were conducted. Specifically, to determine the effects of serum, LY294002, a PI 3-kinase inhibitor, and genistein on Akt/PKB phosphorylation, Mia PACA-2 cells were cultured for 72 hours in the absence or presence of serum with or without 100 μM genistein (GN) or 50 μM LY294002. Western blots were performed on whole cell lysates as described above except that specific antibodies against phosphorylated and total Akt/PKB were used (Akt/PKB is Anti-pS473 AktpAB from Promega, Madison Wis. and total Akt is Akt 1/2 from Santa Cruz, Santa Cruz, Calif.). The membranes were then stripped and re-probed with an antibody against total Akt.

To determine the effects of LY29400 and DPI on NF-κB activation, Mia PACA-2 cells were cultured for 72 hours in the presence of serum and 15 μM DPI with or without 50 μM LY294002. NF-κB DNA binding activity was measured in nuclear extracts by gel shift assay as described above.

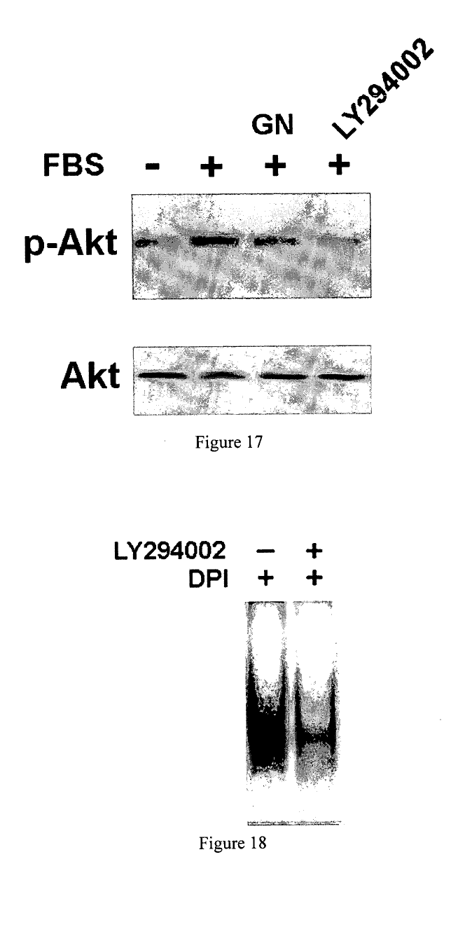

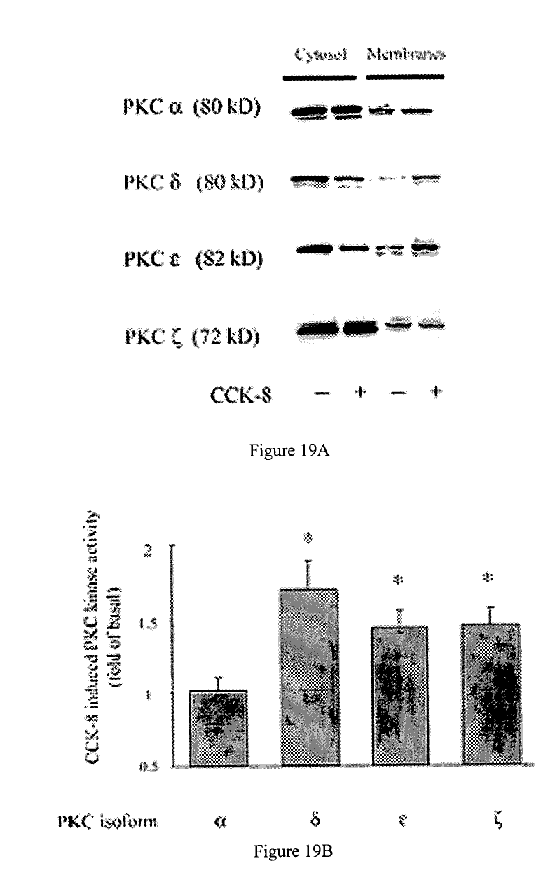

As shown in FIG. 17, serum increases the activated phosphorylated state of Akt/PKB, and LY294002 prevents the serum activation. Additionally, genistein attenuated serum-induced Akt phosphorylation/activation. As shown in FIG. 18, the combination of LY294002 and DPI inhibits NF-κB activation in a manner similar to the combination of a polyphenolic compound and DPI. These results indicate that polyphenolic compounds inhibit NF-κB activation through their effects on PI 3-kinase.

### EXAMPLE 9

### Rottlerin

A. Oligonucleosomal DNA Fragmentation and Rottlerin

The effect of rottlerin and two inhibitors of PKC, GF109203X (GF) and Ro-32-0432 (Ro), on apoptosis in MIA PaCa-2 and PANC-1 pancreatic cancer cells was determined by measuring their effects on oligonucleosomal DNA fragmentation, a specific measure of apoptosis. The effects of the two protein kinase C inhibitors was measured because rottlerin is a known inhibitor of the PKC6 isoform, which both GF and Ro are also known to inhibit.

MIA PaCa-2 cells were cultured for up to about 72 hours in the presence of serum with or without rottlerin (Rt) and protein kinase C inhibitors, GF109203X (GF) and Ro-32-0432 (Ro). Oligonucleosomal DNA fragmentation was measured by an ELISA technique using Cell Death Detection ELISA Plus (Roche, Indianapolis, Ind.). FIG. 29 represents three experiments with similar results.

As shown in FIG. 29, rottlerin caused a marked increase in DNA fragmentation and thus apoptosis with an increasing effect over about 72 hours. In contrast, the PKC inhibitors did not, thereby indicating that rottlerin causes apoptosis in a manner that is independent of its effect on PKC.

PANC-1 cells were cultured for up to about 48 hours in the presence of serum with or without rottlerin (Rt) and protein kinase C inhibitors, GF109203X (GF) and Ro-32-0432 (Ro). Oligonucleosomal DNA fragmentation was measure by an ELISA technique using Cell Death Detection ELISA Plus (Roche, Indianapolis, Ind.). FIG. 30 represents two experiments with similar results.

As shown in FIG. 30, the effects with rottlerin were similar in causing oligonucleosomal DNA fragmentation and thus apoptosis in a second in pancreatic cancer cell line, PANC-1. Again, the PKC inhibitors did not have this effect.

B. Annexin V Staining

Both Annexin V and propidium iodide staining was measured according to Example 3B above using methods known in the art. Annexin V stains phosphatidylserine on the surface of the cell when the cell is impermeant. This is a specific measure of apoptosis because during apoptosis, phosphatidylserine externalizes to the outside surface of the cell. The propidium iodide enters the cell and stains the nucleus when the cell is permeant as happens during necrosis. Cells that stain with Annexin V but not propidium iodide are apoptotic.

MIA PaCa-2 cells were cultured for about 72 hours in the presence of serum with or without the indicated concentrations of rottlerin (Rt). Phosphatidylserine externalization was measured by flow cytometry in cells stained with AnV and PI. AnV⁺/PI⁻ cells were considered apoptotic. Cells positive for PI were considered necrotic. FIG. 31 represents two experiments with similar results. The results indicate that rottlerin causes cell death through apoptosis and not necrosis.

C. Caspase-3 Activity and Oligonucleosomal DNA Fragmentation and Rottlerin

The effects of rottlerin and a caspase inhibitor (Z-VAD) on caspase-3 activity (DEVDase activity) and oligonucleosomal DNA fragmentation in MIA PaCa-2 pancreatic cancer cells were determined. MIA PaCa-2 cells were cultured for 48 hours in the presence of serum with or without the indicated concentrations of rottlerin (Rt), Ro-32-0432 (Ro) and 100 μM z-VAD.fmk (Z-VAD). DEVDase activity was measured in whole cell lysates with a fluorimetric assay as Example 41 above using methods known in the art. DNA fragmentation was measured in cell lysates by cell death ELISA by using Cell Death Detection ELISA Plus (Roche, Indianapolis, Ind.). FIG. 32 represents two experiments with similar results. The results demonstrate that rottlerin caused a dose-dependent and marked increase in the caspase activity and DNA fragmentation. The effect of rottlerin on DNA fragmentation was blocked by the capase inhibitor. These results demonstrate that rottlerin causes apoptosis through activating caspase-3.

D. Mitochondiral Membrane Potential and Rottlerin

The effects of rottlerin on mitochondrial membrane potential in MIA PaCa-2 pancreatic cancer cells was studied. MIA PaCa-2 cells were cultured for 72 hours in the presence of serum. FIG. 33A is a histogram that shows changes in Δψm were measured by flow cytometry in cells labeled with membrane potential sensitive fluorescent dye DiOC₆(3) as provided in Example 51 above using methods known in the art. FIG. 33B shows the percentage of cells with high Δψm. These figures represent two experiments with similar results. The results demonstrate that rottlerin causes marked mitochondrial depolarization.

E. Mitochondrial Cytochrome C Release and Rottlerin

The effects of rottlerin on mitochondrial cytochrome c release in MIA PaCa-2 pancreatic cancer cells was studied. MIA PaCa-2 cells were cultured for about 72 hours in the absence or presence of the indicated concentration of rottlerin. The cells were then lysed and cytosolic fractions were isolated. The cytochrome c level in cytosolic fractions was measured by Western blot analysis. The membranes were stripped and re-probed with an antibody against actin to show equal protein loading. FIG. 34 represents two experiments with the similar results. The results demonstrate that rottlerin causes significant release of mitochondrial cytochrome c into the cytoplasm of the cancer cells. The combination of results indicate that rottlerin causes apoptosis by causing mitochondrial depolarization leading to cytochrome c release which, in turn, leads to caspase-3 activation and apoptosis.

F. NF-κB Activation and ROS Production and Rottlerin

The effects of rottlerin and GF109203X on NF-κB activation in MIA PaCa-2 pancreatic cancer cells was studied. MIA PaCa-2 cells were cultured for about 72 hours in the presence of serum with or without rottlerin (Rt, 2.5 μM) or GF109203X (GF, 10 EM). NF-κB binding activity was measured in nuclear extracts by gel shift assay on the MIA PaCa-2 cells at the end of the incubation as described in Section C above. FIGS. 35 and 36 represent three experiments with similar results. The results show that NF-κB is constitutively activated in the cancer cells and that this activation is blocked by rottlerin treatment but not by GF treatment suggesting that NF-κB activation in cancer cells is not due to PKC activation alone and that rottlerin has effects in addition to its effects on PKC to block NF-κB activation in the cancer cells. The results also indicate that rottlerin promotes mitochondrial changes, caspase-3 activation and apoptosis in part because it inhibits NF-κB activation in the cancer cells.

Effects of rottlerin (Rt) and GF109203X (GF) on production of ROS in MIA PaCa-2 pancreatic cancer cells was studied. MIA PaCa-2 cells were cultured for 72 hours in the presence of serum and with or without rottlerin or GF109203X. Intracellular ROS was measured using oxidation-sensitive cell-permeable fluorescent probe, dichlorofluorescein diacetate (DCF-DA) to measure H₂O₂ using methods known in the art. See Royall & Ischiropoulos (1993) Arch. Biochem. Biophys. 302:348-355, which is herein incorporated by reference. To measure ROS, cells were collected after incubation, washed with PBS, and incubated for 15 minutes with 8 mM DCF-DA. Samples were analyzed by flow cytometry. The amount of DCF-DA fluorescence correlated with the amount of ROS in the cells. FIG. 36B shows the percentage of cells with high DCF fluorescence. These figures represent two experiments with the similar results. The results demonstrate that rottlerin almost completely inhibits ROS production in the cancer cells. These results indicate that rottlerin promotes mitochondrial changes, caspase-3 activation and apoptosis in part because it inhibits ROS generation in the cancer cells.

G. MIA PaCa-2 Tumor Growth In Vivo and Rottlerin

The effect of rottlerin on the growth of MIA PaCa-2 tumors in nude mice was studied. MIA PaCa-2 cells were injected subcutaneously into the flank of nude mice. Animals were thereafter treated with daily intraperitoneal injections of either rottlerin (0.5 mg/kg body weight) or control vehicle for 14 days. Tumor volume was assessed at the end of the treatment period using the formula for a hemi-ellipsoid (2/3*π*a*b*c with a, b, and c being the half diameters for height, width, and length of the tumor). After a 14 days treatment period rottlerin caused a significant reduction in tumor growth with an average tumor volume of about 10.21 mm³, while control tumors reached an average size of about 30.88 mm³. This translates to about a 67% reduction in tumor volume after 14 days as shown in FIG. 37, represents the mean of 4 tumors in each group. These experiments demonstrate that rottlerin in addition to the proapoptotic effects in vitro also exhibits potent anti-tumor effects in animals.

H. Metabolic Profiling

The tracer for this metabolic profiling study, stable isotope [1,2-¹³C₂]-D-glucose, was purchased with greater than about 99% purity and about 99% isotope enrichment for each position from Cambridge Isotope Laboratories, Inc., Andover, Mass.

Seventy-five percent confluent cultures of MIA PaCa-2 cells were incubated in [1,2-13C₂]D-glucose-containing media (100 mg/dl total concentration=5 mM; 50% isotope enrichment, i.e. half unlabeled glucose, half labeled with the stable isotope ¹³C tracer). Cells were plated at a density of about 1x06 cells per 75 ml culture flask. In separate experiments MIA cells were treated with graded doses of Rottlerin (2.5 and 5 μM). Glucose and lactate levels in the medium were measured using a Cobas Mira chemistry analyzer (Roche Diagnostics, Pleasanton, Calif., USA).

I. RNA and DNA Synthesis and Rottlerin

RNA ribose and DNA deoxyribose were isolated by acid hydrolysis of cellular nucleic acid after Trizol purification of cell extracts. Total RNA amounts were assessed by spectrophotometric determination, in triplicate cultures. Ribose was derivatized to its aldonitrile acetate form using hydroxylamine in pyridine with acetic anhydride (Supelco, Bellefonte, Pa.) before mass spectral analyses. The ion cluster was monitored around the m/z 256 (carbons 1-5 of ribose) (chemical ionization, CI) and m/z 217 (carbons 3-5 of ribose) and m/z 242 (carbons 1-4 of ribose) (electron impact ionization, EI) to determine molar enrichment and the positional distribution of 1³C in ribose. By convention, the base mass of 1²C— compounds (with their deriviatization agents) is given as m₀ as measured by mass spectrometry as described in the prior art. See Boros et al. (2002) Drug Discovery Today 7:364-372, which is herein incorporated by reference. FIG. 38 shows total tracer ¹³C carbon incorporation into DNA deoxyribose and RNA ribose, respectively, from the tracer [1,2-¹³C₂]glucose. There was a dose-dependent decrease in DNA labeling and a well-maintained rate of RNA synthesis indicating a strong cell cycle arrest in MIA cells after increasing doses of rottlerin treatment.

J. Oxidative and Non-Oxidative Deoxyribose and Ribose Synthesis and Rottlerin

The effect of rottlerin on oxidative deoxyribose deoxyribose and non-oxidative synthesis, as well as oxidative ribose and non-oxidative ribose synthesis based on positional ¹³C tracer accumulation from glucose into nucleic acid of MIA PaCa-2 cells was studied. MIA PaCa-2 cells were treated with vehicle, 2.5 μM and 5.0 μM rottlerin for 72 hours in the presence of [1,2-¹³C₂]glucose in culture. Deoxyribose and ribose molecules labeled with a single ¹³C atom on the first carbon position (ml) recovered from DNA or RNA were used to guage the ribose fraction produced by direct oxidation of glucose through the G6PD pathway. Deoxyribose and ribose molecules labeled with ¹³C on the first two carbon positions (m2) were used to measure the fraction produced by the non-oxidative steps of the pentose cycle via transketolase. Rottlerin induced a significant decrease in non-oxidative deoxyribose synthesis (2) with a compensatory increase in oxidative deoxyribose synthesis (1), but rottlerin affected neither oxidative (3) nor non-oxidative (4) RNA ribose synthesis. The primary effect of rottlerin on MIA PaCa-2 cells is the selective inhibition of DNA precursor synthesis via the non-oxidative steps of the pentose cycle involving transketolase and transaldolase as target enzymes. FIG. 39 represents the data of 3 cultures in each group.

J. Lactate Production and Rottlerin

Lactate from the cell culture media (0.2 ml) was extracted by ethylene chloride after acidification with HCL. Lactate was derivatized to its propylamine-HFB form and the m/z328 (carbons 1-3 of lactate) (chemical ionization, CI) was monitored for the detection of ml (recycled lactate through the PC) and m2 (lactate produced by the Embden-Meyerhof-Parnas pathway) for the estimation of pentose cycle activity. See Lee et al. (1998) Am. J. Physiol. 274:E843-E851, which is herein incorporated by reference. MIA PaCa-2 cells were treated with vehicle, 2.5 μM and 5.0 μM rottlerin for 72 hours in the presence of [1,2-13C₂]glucose in culture. Rottlerin decreased direct glucose oxidation and recycling in the pentose cycle, although this effect was not dose dependent. FIG. 40 represents the mean of 3 cultures in each group. The ml/m2 ratios in lactate produced and released by MIA PaCa-2 pancreatic adenocarcinoma cells was recorded in order to determine pentose cycle activity versus glycolysis in response to rottlerin treatment. The data in FIG. 41 demonstrate that cultured MIA PaCa-2 cells oxidize about 1.6 percent of glucose via the pentose cycle then recycle this substrate back to glycolysis via transketolase and transaldolase. Rottlerin decreased direct glucose oxidation in the pentose cycle and recycling via the non-oxidative steps of the pentose cycle, although this decrease was not dose dependent.

K. Anaplerotic Flux and Rottlerin

Glutamate label distribution from glucose is suitable for determining glucose oxidation versus anabolic glucose use within the TCA cycle, also known as anaplerotic flux. See Lee et al. (1996) Dev. Neurosci. 18:469-477, which is herein incorporated by reference. In order to measure glutamate, tissue culture medium was first treated with 6% perchloric acid. The supernatant was passed through a 3 cm³ Dowex-50 (H+) column. Amino acids were eluted with 15 ml 2N ammonium hydroxide. To further separate glutamate from glutamine, the amino acid mixture was passed through a 3 cm³ Dowex-1 (acetate) column, then collected with 15 ml 0.5 N acetic acid. The dry glutamate fraction from tissue culture medium was converted to its trifluoroacetyl butyl ester (TAB). Under EI conditions, ionization of TAB-glutamate gives rise to two fragments, m/z198 and m/z152, corresponding to C₂-C₅ and C₂-C₄ of glutamate. See Leimer et al. (1977) J. Chromatogr. 141:121-144, which is herein incorporated by reference. Glutamate labeled on the 4-5 carbon positions indicates pyruvate dehydrogenase activity while glutamate labeled on the 2-3 carbon positions indicates pyruvate carboxylase activity for the entry of glucose carbons to the TCA cycle. TCA cycle anabolic glucose utilization is calculated based on the m₁/m₂ ratios of glutamate. The TCA cycle metabolite alpha-ketoglutarate is in equilibrium with glutamate, which is released by the cells into the medium. The m2/m1 ratio in glutamate is proportional with the activity of glucose oxidation as ¹³CO₂ is released from alpha-ketoglutarate during each completed cycle. TCA cycle anaplerotic flux is calculated based on the m2/m1 ratios of glutamate.

Anaplerosis refers to the reactions that allow the entry of carbon into the TCA cycle intermediate pools other than via citrate synthase. Any carbon that enters the cycle as acetyl-CoA is oxidized to carbon dioxide and water; any carbon that enters the citric acid cycle via an anaplerotic pathway is not oxidized, but must be disposed of by some other route. Glutamate dehydrogenase is one possible route providing equilibrium between alpha-ketoglutarate and glutamate, some other reactions include pyruvate carboxylation, transamination reactions and propionate carboxylation.

MIA PaCa-2 cells were treated with vehicle, 2.5 μM and 5.0 μM rottlerin for 72 hours in the presence of [1,2-1³C₂]glucose in culture. Rottlerin increased glucose oxidation in the TCA cycle, which indicates that MIA cells utilize glucose for energy production more efficiently in the presence of rottlerin. Glucose substrate flow in rottlerin treated MIA cells is from nucleotide synthesis toward glucose oxidation and energy production in the TCA cycle. These rottlerin-induced changes in metabolism prevent the cancer cell from rapidly proliferating and make them susceoptible to apoptosis. The data in FIG. 41 represent the mean of 3 cultures in each group.

L. De Novo Fatty Acid Synthesis and Rottlerin

The effect of rottlerin on de novo myrystate, palmitate, stearate and oleate fatty acid synthesis of MIA PaCa-2 cells was studied. MIA PaCa-2 cells were treated with vehicle, 2.5 μM and 5.0 μM rottlerin for 72 hours in the presence of [1,2-¹³C₂]glucose in culture. Rottlerin induced a significant sharp decrease in the de novo synthesis of all fatty acid species. The data in FIG. 42 represent the mean of 3 cultures in each group. Myrystate (C:14), palmitate (C:16), stearate (C:18) and oleate (C:18-1) were extracted after saponification of cell pellets in 30% KOH and 100% ethanol using petroleum ether. Fatty acids were converted to their methylated derivative using 0.5N methanolic-HCL. Palmitate, stearate and oleate were monitored at m/z 270, m/z 298 and m/z 264, respectively, with the enrichment of ¹³C labeled acetyl units which reflect synthesis, elongation and desaturation of the new lipid fraction as determined by mass isotopomer distribution analysis (MIDA) of different isotopomers. See Lee et al. (1995) Anal. Biochem. 226:100-112, and Lee et al. (1998) J. Biol. Chem. 273:20929-20934, which are herein incorporated by reference.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A method of treating, preventing, inhibiting, or modulating NF-κB activation in a cell or in a subject which comprises administering at least one polyphenolic compound, an inhibitor of PKC 8 translocation, an inhibitor of PKC ε translocation, or a combination thereof to the cell or the subject.

2. The method of claim 1, wherein the polyphenolic compound is rottlerin or a derivative thereof.

3. The method of claim 1, which further comprises administering a second polyphenolic compound to the cell or the subject.

4. The method of claim 3, wherein the second polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid and traxol.

5. The method of claim 4, wherein the polyphenolic compound is quercetin, rutin, genistein, curcumin or trans-resveratrol.

6. The method of claim 1, which further comprises administering at least one inhibitor of a reactive oxygen species to the cell or the subject.

7. The method of claim 6, wherein the inhibitor is diphenylene iodonium, N-acetylcysteine, or Tiron.

8. The method of claim 1, which further comprises administering at least one antioxidant to the cell or the subject.

9. The method of claim 1, wherein the inhibitor of PKC δ translocation or the inhibitor of PKC ε translocation is a peptide.

10. The method of claim 9, wherein the peptide is δV1-1 or εV1-2.

11. A method of treating, preventing, or inhibiting a disease or disorder associated with NF-κB activation in a subject which comprises conducting the method of claim 1.

12. The method of claim 11, wherein the disease or disorder is a cancer.

13. The method of claim 12, wherein the cancer is pancreatic cancer, breast cancer, ovarian cancer, prostate cancer, kidney cancer, pancreatic cancer, colon cancer, thyroid cancer, melanoma, Hodgkin's lymphoma, acute lymphoblastic leukemia, acute myelogenous leukemia, diffuse large B-cell lymphoma, astrocytoma, glioblastoma, a head or neck cancer, or vulva cancer.

14. The method of claim 12, wherein the cancer is related to in vitro transformation of BCR-ABL, DBL/DBS, RAF, RAS, TEL-JAK2, or TEL-PDGFR.

15. The method of claim 12, wherein the cancer is related to viral oncogenesis caused by Epstein-Barr virus, hepatitis B virus, human herpesvirus-8, or human T-cell leukemia virus-1.

16. The method of claim 11, wherein the disease or disorder is an inflammatory disease.

17. The method of claim 16, wherein the inflammatory disease is pancreatitis, inflammatory bowel disease, asthma, arthritis, rheumatoid arthritis, asthma, psoriasis, cystitis, or nephritis.

18. The method of claim 11, wherein the disease or disorder is viral hepatitis, alcoholic liver disease, lung inflammation, Alzheimer's Disease, or atherosclerosis.

19. The method of claim 11, which further comprises administering at least one antiproliferative agent, at least one anti-inflammatory agent, or both.

20. The method of claim 12, wherein administration of the polyphenolic compound, the inhibitor of PKC δ translocation, the inhibitor of PKC ε translocation, or the combination thereof causes, induces, increases, or modulates cell cycle arrest, apoptosis, mitochondrial cytochrome c release, dissipation of mitochondrial polarity, caspase activation, mitochondrial permeability transition pore activation, or a combination thereof, in the cancer.

21. The method of claim 12, which further comprises administering a second polyphenolic compound, at least one inhibitor of reactive oxygen species, at least one inhibitor of PI 3-kinase, at least one inhibitor of NADPH oxidase, or a combination thereof.

22. The method of claim 21, wherein administration of the second polyphenolic compound, the inhibitor of reactive oxygen species, the inhibitor of PI 3-kinase, the inhibitor of NADPH oxidase, or the combination thereof causes, induces, increases, or modulates cell cycle arrest, apoptosis, mitochondrial cytochrome c release, dissipation of mitochondrial polarity, caspase activation, mitochondrial permeability transition pore activation, or a combination thereof, in the cancer.

23. The method of claim 12, wherein the polyphenolic compound is rottlerin.

24. The method of claim 23, wherein rottlerin inhibits non-oxidative deoxyribose synthesis, inhibits nucleic acid synthesis, induces cell cycle arrest, inhibits cell proliferation, increases oxidative metabolism of glucose, inhibits de novo fatty acid synthesis, chain elongation and desaturation from glucose, or a combination thereof, in the cancer.

25. A method of inducing apoptosis in a cell or making the cell susceptible to apoptosis which comprises conducting the method of claim 1.

26. The method of claim 25, wherein the cell is a tumor cell or a cancer cell.

27. The method of claim 26, wherin the tumor is a primary tumor.

28. The method of claim 26, wherein the tumor is metastatic.

29. A method of inhibiting non-oxidative deoxyribose synthesis, inhibiting nucleic acid synthesis, inducing cell cycle arrest, inhibiting cell proliferation, increasing oxidative metabolism of glucose, inhibiting de novo fatty acid synthesis, chain elongation and desaturation from glucose, or a combination thereof, in a cell which comprises contacting the cell with rottlerin.

30. A purified polypeptide sequence comprising SFNSYELGSLRQIKIWFQNRRMKWKK (SEQ ID NO:10), EAVSLKPTRQIKIWFQNRRMKWKK (SEQ ID NO:11), or LSETKPAVRQIKIWFQNRRMKWKK (SEQ ID NO:12).

31. A pharmaceutical composition comprising rottlerin and a pharmaceutically acceptable carrier.

32. The pharmaceuctical composition of claim 31, and further comprising a second polyphenolic compound.

33. The pharmaceutical composition of claim 31, and further comprising an inhibitor of PKC δ translocation, an inhibitor of PKC ε translocation, or both.

34. The pharmaceutical composition of claim 31, and further comprising an inhibitor of a reactive oxygen species.

35. The pharmaceutical composition of claim 31, and further comprising an antioxidant.


SEQUENCE LISTING (12 sequences)

SEQ ID NO: 1 - protein, 10 aa, Artificial sequence
  a PKC d translocation inhibitor
SFNSYELGSL

SEQ ID NO: 2 - protein, 8 aa, Artificial sequence
  a PKC epsilon translocation inhibitor
EAVSLKPT

SEQ ID NO: 3 - protein, 8 aa, Artificial sequence
  Control peptide
LSETKPAV

SEQ ID NO: 4 - protein, 16 aa, Drosophila melanogaster
RQIKIWFQNRRMKWKK

SEQ ID NO: 5 - DNA, 20 bp, Artificial sequence
  Probe sequence
gcagaggggactttccgaga

SEQ ID NO: 6 - protein, 11 aa, Artificial sequence
  Substrate for PKC alpha isoform
QKRPSQRSKYL

SEQ ID NO: 7 - protein, 22 aa, Artificial sequence
  Substrate for PKC delta isoform
RFAVRDMRQTVAVGVIKAVDKK

SEQ ID NO: 8 - protein, 16 aa, Artificial sequence
  Substrate for PKC epsilon isoform
ERMRPRKRQGSVRRRV

SEQ ID NO: 9 - protein, 13 aa, Artificial sequence
  Substrate for PKC z isoform
SIYRRGSRRWRKL

SEQ ID NO: 10 - protein, 26 aa, Artificial sequence
  PKC inhibitor cell permeable
SFNSYELGSLRQIKIWFQNRRMKWKK

SEQ ID NO: 11 - protein, 24 aa, Artificial sequence
  PKC inhibitor cell permeable
EAVSLKPTRQIKIWFQNRRMKWKK

SEQ ID NO: 12 - protein, 24 aa, Artificial sequence
  Control cell permeable peptide
LSETKPAVRQIKIWFQNRRMKWKK